(19) United States
(12) Patent Application Publication
(10) Pub. No.: US 2008/0207705 A1
(43) Pub. Date: Aug. 28, 2008

(54) Heteroaryl-Imidazole Derivatives as Cannabinoid CB1 Receptor Antagonists

(75) Inventors: Jinhwa LEE, Yongin-si (KR); Jeongmin Kim, Seoul (KR); Jong Yup Kim, Yongin-si (KR); Hee Jeong Seo, Seoul (KR); Myung Eun Jung, Seongnam-si (KR); Sung-Han Lee, Seoul (KR); Kwangwoo Ahn, Dobong-gu (KR); Suk Ho Lee, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: Green Cross Corporation, Yongin-si (KR)

(21) Appl. No.: 12/037,986

(22) Filed: Feb. 27, 2008

Related U.S. Application Data
(63) Continuation-in-part of application No. 11/679,742, filed on Feb. 27, 2007.

(51) Int. Cl. A61K 31/4245; C07D 413/04; A61K 31/433; A61K 31/426; A61P 3/04; A61K 31/421; C07D 417/04

(52) U.S. Cl. 514/363; 548/143; 548/137; 548/235; 548/202; 514/364; 514/374; 514/365

(57) ABSTRACT

A novel heteroaryl-imidazole compound of formula (I) or a pharmaceutically acceptable salt thereof is effective as a cannabinoid CB₁ receptor inverse agonist or antagonist, which is useful for preventing or treating obesity and obesity-related metabolic disorders. The present invention also provides a method for preparing same, a pharmaceutical composition containing same, and a method for preventing or treating obesity and obesity-related metabolic disorders.

### FIELD OF THE INVENTION

The present invention relates to a novel heteroaryl-imidazole compound which is effective as a cannabinoid CB₁ receptor inverse agonist or antagonist.

### DESCRIPTION OF THE PRIOR ART

The World Health Organization (WHO) recently reported that obesity has become a global epidemic, posing a serious threat to public health because of the increased risk of associated health problems (See Report of a WHO Consultation on Obesity: Obesity-Preventing and Managing a Global Epidemic; World Health Organization: Geneva, 1997). Obesity is characterized by excess body fat, especially visceral fat, and constitutes a pro-inflammatory state eventually leading to serious health consequences. There are growing evidences that obesity as a chronic disease cannot be cured by short-term dieting or exercise alone, but additional pharmacological treatments would lead to higher success rates.

CB₁ cannabinoid receptor belongs to G-protein-coupled receptor (GPCR) type and is coupled to inhibitory G proteins (G(i/o)) to inhibit certain adenylyl cyclase isozymes, leading to decreased cAMP production, decreased Ca²⁺ conductance, increased K⁺conductance, and increased mitogen-activated protein kinase activity (See Di Marzo et al., Nat. Rev. Drug Discovery 2004, 3, 771-784; Rhee, M. H. et al., J. Neurochem. 1998, 71, 1525-1534). The major physiological effect of cannabinoids (in the central nervous system (CNS) and neuronal tissues) is the modulation of neurotransmitter release via activation of presynaptic CB₁ receptors located on distinct types of axon terminals throughout the brain (See Howlett, A. C. et al., Neuropharmacology 2004, 47 (Suppl. 1), 345-358).

The CB₁ receptor is mainly expressed in several brain areas including the limbic system (amygdala, hippocampus), hypothalamus, cerebral cortex, cerebellum, and basal ganglia. In the cerebellum and basal ganglia, cannabinoids modulate the locomotor activity. In the limbic system, cannabinoids influence learning, memory, emotion, and motivation, and through activation of CB₁ receptors in the limbic system-hypothalamus axis, cannabinoids have an important role in the control of appetite. Moreover, lower levels of CB₁ receptors can also be found in peripheral tissues including urinary bladder, testis, prostate, GI tract, heart, lung, adrenal gland, parotid gland, bone marrow, uterus, ovary, and adipose tissue (See Cota, D. et al., J. Clin. Invest. 2003, 112, 423-431; Ravinet Trillou, C. et al., Int. J. Obes. Relat. Metab. Disord. 2004, 28, 640-648; Galiegue, S. et al., Eur. J. Biochem. 1995, 232, 54-61; Howlett, A. C. et al., Pharmacol. Rev. 2002, 54, 161-202).

Many preclinical in vitro and in vivo experiments have been shown that CB₁ receptor antagonists can influence energy homeostasis by central and peripheral mechanisms and may represent promising targets to treat diseases that are characterized by impaired energy balance. Already the first published studies with rimonabant (SR141716) in both rodents (See Arnone, M. et al., Psychopharmacology (Berlin) 1997, 132, 104-106) and primates (See Simiand, J.; Keane, M.; Keane, P. E.; Soubrie, P. Behav. Pharmacol. 1998, 9, 179-181) showed clear differentiation, i.e., marked effects on sweet food intake versus marginal effects on regular chow intake or water drinking. Many other preclinical “proof of concept” studies have been performed in the meantime with several CB agonists and antagonists to further uncover the amount and mode of contribution of cannabinergic system modulators to energy homeostasis. Almost all of those studies have been recently reviewed (See Smith, R. A. et al., IDrugs 2005, 8, 53-66).

Considering the important impact of obesity on public health and the lack of any efficient and viable drug to cure it, it is no surprise that CB₁ antagonists are currently the subject of intense studies, which were published in several reviews (See Adam, J. et al., Expert Opin. Ther. Patents, 2002, 12(10), 1475-1489; Hertzog, D. L. Expert Opin. Ther. Patents, 2004, 14(10), 1435-1452; Lange, J. H. M. et al., Drug Discov. Today, 2005, 10, 693-702; Bishop, M. J. J Med. Chem., 2006, 49(14), 4008-4016).

### SUMMARY OF THE INVENTION

It is a primary object of the present invention to provide a novel heteroaryl-imidazole compound of formula (I) or a pharmaceutically acceptable salt thereof, which is effective as a cannabinoid CB₁ receptor inverse agonist or antagonist, useful for preventing or treating obesity and obesity-related metabolic disorders.

It is another object of the present invention to provide a method for preparing the inventive compound.

It is another object of the present invention to provide a pharmaceutical composition for preventing or treating obesity and obesity-related metabolic disorders, comprising the inventive compound as an active ingredient.

### DETAILED DESCRIPTION OF THE INVENTION

In accordance with one aspect of the present invention, there is provided a compound of formula (I) or a pharmaceutically acceptable salt thereof and a method for preparing same:

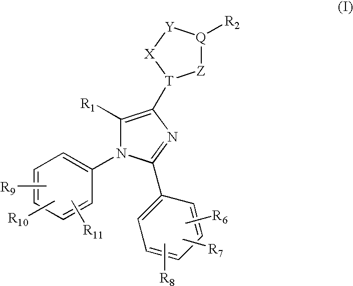

wherein:

R₁ is hydrogen, C₁₋₅ alkyl, substituted C₁₋₅ alkyl, C₂₋₄ alkenyl, substituted C₂₋₄ alkenyl, C₂₋₄ alkynyl, substituted C₂₋₄ alkynyl, halogen, (CH₂)ₙ—C₃₋₅ carbocycle or (CH₂)n-heterocycle, n being 0 or 1;

R₂ is hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, C₃₋₆ carbocycle-R₅ or (CH₂)ₘ—R₅, m being 1 for 2;

R₃ and R₄ are each independently hydrogen, C₁₋₆ alkyl, substituted C₁₋₆ alkyl, C₂₋₆ alkenyl, substituted C₂₋₆ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl; or

R₃ and R₄, together with the nitrogen atom to which they are bonded, form a 4- to 10-membered saturated or unsaturated heterocyclic ring which is optionally substituted by one or more C₁₋₃ alkyl, benzyl, phenyl, C₁₋₃ alkoxy or halogen;

R₅ is heteroalkyl, phenyl, furanyl, benzofuranyl, thienyl, benzothienyl, pyridinyl, pyrimidinyl, pyrazinyl, pyridizinyl, tetrahydrofuranyl, tetrahydropyranyl, dioxanyl, 1,4-benzodioxanyl or benzo[1,3]dioxolyl, each being optionally substituted by one or more groups consisting of halogen, C₁₋₃ alkyl and C₁₋₂ alkoxy, each having optional one to three fluorine substitutents;

R₆, R₇, R₈, R₉, R₁₀ and R₁₁ are each independently hydrogen, halogen, C₁₋₃ alkyl, C₁₋₃ alkoxy or trifluoromethyl;

X, Y and Z are each independently selected from the group consisting of —C(R₁₂)═, —O—, —N═, —N(R₁₃)— and —S— to form an aromatic heterocycle together with Q and T;

Q and T are each independently

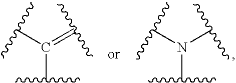

with the proviso that both Q and T can not be simultaneously

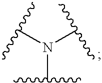

and

R₁₂ and R₁₃ are each independently hydrogen, carbocycle, substituted carbcycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, C₂₋₆ alkynyl optionally substituted by alkoxy or halogen, (CH₂)ₘ—C_{3 6} carbocycle optionally substituted by alkoxy or halogen, or (CH₂)ₘ—R₅, m being 1 or 2, and R₅ having the same meaning as defined above.

The aromatic heterocycles formed by X, Y, Z, Q and T encompass, for example, oxazole, isoxazole, thiazole, isothiazole, triazole, oxadiazole, thiadiazole and tetrazole.

As used herein, the term “alkyl” refers to a straight or branched chain saturated hydrocarbon radical. Examples of “alkyl” as used herein include, but are not limited to, methyl, ethyl, n-propyl, isopropyl, n-butyl, isobutyl, t-butyl, n-pentyl, isopentyl and hexyl.

As used herein, the term “substituted alkyl” refers to a straight or branched chain saturated hydrocarbon radical, which is optionally substituted by one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents, sulfanyl, sulfinyl, sulfonyl, oxo, hydroxy, mercapto, amino, guanidino, carboxy, aminocarbonyl, aryl, aryloxy, heteroaryl, heteroaryloxy, heterocyclic, aminosulfonyl, sulfonylamino, carboxyamide, ureido, nitro, cyano and halogen.

As used herein, the term “alkenyl” refers to a straight or branched chain hydrocarbon radical having at least one carbon-carbon double bond. Examples of “alkenyl” as used herein include, but are not limited to, ethenyl and propenyl.

As used herein, the term “substituted alkenyl” refers to a straight or branched chain hydrocarbon radical having at least one carbon-carbon double bond, which has optional substituents selected from the group consisting of C₁₃ alkyl optionally having one to three fluorine substituents, amino, aryl, cyano and halogen.

As used herein, the term “alkynyl” refers to a straight or branched chain hydrocarbon radical having at least one carbon-carbon triple bond. Examples of “alkynyl” as used herein include, but are not limited to, acetylenyl and 1-propynyl.

As used herein, the term “substituted alkynyl ” refers to a straight or branched chain hydrocarbon radical having at least one carbon-carbon triple bond, optionally having one or more substituents selected from the group consisting of C₁₋₃ alkyl optionally having one to three fluorine substituents, amino, aryl and halogen.

As used herein, the term “halogen” refers to fluorine (F), chlorine (Cl), bromine (Br), or iodine (I).

As used herein, the term “carbocycle” refers to a non-aromatic cyclic hydrocarbon radical composed of three to seven carbon atoms. Five-to seven-membered rings may contain a double bond in the ring structure. Exemplary “carbocycle” groups include, but are not limited to, cyclopropyl, cyclobutyl, cyclopentyl, cyclopentenyl, cyclohexyl, and cycloheptyl.

As used herein, the term “substituted carbocycle” refers to a non-aromatic cyclic hydrocarbon radical composed by three to seven carbon atoms, which is optionally substituted with one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents, sulfanyl, sulfinyl, sulfonyl, oxo, hydroxy, mercapto, amino, guanidino, carboxy, aminocarbonyl, aryl, aryloxy, heteroaryl, heterocyclic, aminosulfonyl, sulfonylamino, carboxyamide, nitro, ureido, cyano and halogen.

As used herein, the term “aryl” refers to an optionally substituted benzene ring or refers to a ring system which may result by fusing one or more optional substituents. Exemplary optional substituents include substituted C₁₋₃ alkyl, substituted C₂₋₃ alkenyl, substituted C₂₋₃ alkynyl, heteroaryl, heterocyclic, aryl, alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, or ureido. Such a ring or ring system may be optionally fused to aryl rings (including benzene rings) optionally having one or more substituents, carbocycle rings or heterocyclic rings. Examples of “aryl” groups include, but are not limited to, phenyl, naphthyl, tetrahydronaphthyl, biphenyl, indanyl, anthracyl or phenanthryl, as well as substituted derivatives thereof.

As used herein, the term “heteroaryl” refers to an optionally substituted monocyclic five to six-membered aromatic ring containing one or more heteroatomic substitutions selected from S, SO, SO₂, O, N, or N-oxide, or refers to such an aromatic ring fused to one or more rings such as heteroaryl rings, aryl rings, heterocyclic rings, or carbocycle rings (e. g., a bicyclic or tricyclic ring system), each having optional substituents.

Examples of optional substituents are selected from the group consisting of substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen or ureido. Examples of “heteroaryl” groups used herein include, but are not limited to, benzoimidazolyl, benzothiazolyl, benzoisothiazolyl, benzothiophenyl, benzopyrazinyl, benzotriazolyl, benzo[1,4]dioxanyl, benzofuranyl, 9H-a-carbolinyl, cinnolinyl, furanyl, furo[2,3-b]pyridinyl, imidazolyl, imidazolidinyl, imidazopyridinyl, isoxazolyl, isothiazolyl, isoquinolinyl, indolyl, indazolyl, indolizinyl, naphthyridinyl, oxazolyl, oxothiadiazolyl, oxadiazolyl, phthalazinyl, pyridyl, pyrrolyl, purinyl, pteridinyl, phenazinyl, pyrazolyl, pyridyl, pyrazolopyrimidinyl, pyrrolizinyl, pyridazyl, pyrazinyl, pyrimidyl, 4-oxo-1,2-dihydro-4H-pyrrolo[3,2,1-ij]-quinolin-4-yl, quinoxalinyl, quinazolinyl, quinolinyl, quinolizinyl, thiophenyl, triazolyl, triazinyl, tetrazolopyrimidinyl, triazolopyrimidinyl, tetrazolyl, thiazolyl, thiazolidinyl, and substituted versions thereof.

As used herein, the term “heterocyclic” refers to a three to seven-membered ring containing one or more heteroatomic moieties selected from S, SO, SO₂, O, N, or N-oxide, optionally substituted with one or more substituents selected from the group which includes substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, and ureido. Such a ring can be saturated or have one or more degrees of unsaturation. Such a ring may be optionally fused to one or more “heterocyclic” ring(s), aryl ring(s), heteroaryl ring(s) or carbocycle ring(s), each having optional substituents.

Examples of “heterocyclic” moieties include, but are not limited to, 1,4-dioxanyl, 1,3-dioxanyl, pyrrolidinyl, pyrrolidin-2-onyl, piperidinyl, imidazolidine-2,4-dionepiperidinyl, piperazinyl, piperazine-2,5-dionyl, morpholinyl, dihydropyranyl, dihydrocinnolinyl, 2,3-dihydrobenzo [1,4]dioxinyl, 3,4-dihydro-2H-benzo[b][1,4]-dioxepinyl, tetrahydropyranyl, 2,3-dihydrofuranyl, 2,3-dihydrobenzofuranyl, dihydroisoxazolyl, tetrahydrobenzodiazepinyl, tetrahydroquinolinyl, tetrahydrofuranyl, tetrahydronaphthyridinyl, tetrahydropurinyl, tetrahydrothiopyranyl, tetrahydrothiophenyl, tetrahydroquinoxalinyl, tetrahydropyridinyl, tetrahydrocarbolinyl, 4H-benzo[1,3]-dioxinyl, benzo[1,3]dioxonyl, 2,2-difluorobenzo-[1,3]-dioxonyl, 2,3-dihydro-phthalazine-1,4-dionyl, and isoindole-1,3-dionyl.

As used herein, the term “alkoxy” refers to the group —ORₐ, where Rₐ is alkyl as defined above. Exemplary alkoxy groups useful in the present invention include, but are not limited to, methoxy, difluoromethoxy, trifluoromethoxy, ethoxy, n-propoxy, isopropoxy, n-butoxy and t-butoxy.

As used herein the term “aralkoxy” refers to the group —ORₐR_{b}, wherein Rₐ is alkyl and R_{b} is aryl as defined above.

As used herein the term “aryloxy” refers to the group —OR_{b}, wherein R_{b} is aryl as defined above.

As used herein, the term “mercapto” refers to the group —SH.

As used herein, the term “sulfanyl” refers to the group —SR_{c}, wherein R_{c} is substituted alkyl, substituted carbocycle, aryl, heteroaryl or heterocyclic, as defined above.

As used herein, the term “sulfinyl” refers to the group —S—(O)R_{c}, wherein R_{c} is substituted alkyl, substituted carbocycle, aryl, heteroaryl or heterocyclic, as defined above.

As used herein, the term “sulfonyl” refers to the group —S(O)₂R_{c}, wherein R_{c} is substituted alkyl, substituted carbocycle, aryl, heteroaryl or heterocyclic, as defined above.

As used herein, the term “oxo” refers to the group ═O.

As used herein, the term “hydroxy” refers to the group —OH.

As used herein, the term “amino” refers to the group —NH₂. The amino group is optionally substituted by substituted alkyl, substituted carbocycle, aryl, heteroaryl or heterocyclic, as defined above.

As used herein, the term “cyano” refers to the group —CN.

As used herein, the term “aminosulfonyl” refers to the group —S(O)₂NH₂. The aminosulfonyl group is optionally substituted by substituted alkyl, substituted carbocycle, aryl, heteroaryl or heterocyclic, as defined above.

As used herein, the term “sulfonylamino” refers to the group —NHS(O)₂R_{c} wherein R_{c} is substituted alkyl, substituted carbocycle, aryl, heteroaryl or heterocyclic, as defined above.

As used herein, the term “carboxyamide” refers to the group —NHC(O)R_{c} wherein R_{c} is substituted alkyl, substituted carbocycle, aryl, heteroaryl or heterocyclic, as defined above.

As used herein, the term “carboxy” refers to the group —C(O)OH. The carboxy group is optionally substituted by substituted alkyl, substituted carbocycle, aryl, heteroaryl or heterocyclic, as defined above.

As used herein, the term “aminocarbonyl” refers to the group —C(O)NH₂. The aminocarbonyl group is optionally substituted by substituted alkyl, substituted carbocycle, aryl, heteroaryl or heterocyclic, as defined above.

As used herein, the term “ureido” refers to the group —NHC(O)NHR_{c} wherein R_{c} is hydrogen, alkyl, carbocycle or aryl as defined above.

As used herein, the term “guanidino” refers to the group —NHC(═NH)NH₂.

As used herein, the term “acyl” refers to the group —C(O)R_{c}, wherein R_{c} is alkyl, carbocycle, or heterocyclic as defined herein.

As used herein, the term “aroyl” refers to the group —C(O)R_{b}, wherein R_{b} is aryl as defined herein.

As used herein, the term “heteroaroyl” refers to the group —C(O)R_{d}, wherein R_{d} is heteroaryl as defined herein.

As used herein, the term “acyloxy” refers to the group —OC(O)R_{c}, wherein R_{c} is alkyl, carbocycle, or heterocyclic as defined herein.

As used herein, the term “aroyloxy” refers to the group —OC(O)R_{b}, wherein R_{b} is aryl as defined herein.

As used herein, the term “heteroaroyloxy” refers to the group —OC(O)R_{d}, wherein R_{d} is heteroaryl as defined herein.

One embodiment of the present invention is to provide a compound of formula (Ia) or a pharmaceutically acceptable salt thereof:

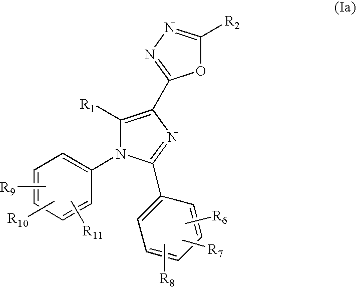

wherein, R₁, R₂, R₆, R₇, R₈, R₉, R₁₀ and R₁₁ have the same meanings as defined above.

Another embodiment of the present invention is to provide a compound of formula (Ib) or a pharmaceutically acceptable salt thereof:

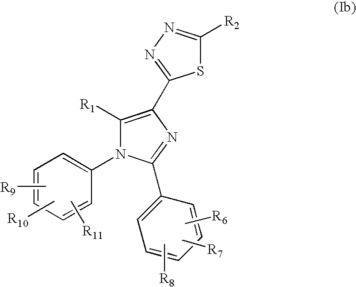

wherein, R₁, R₂, R₆, R₇, R₈, R₉, R₁₀ and R₁₁ have the same meanings as defined above.

A further embodiment of the present invention is to provide a compound of formula (Ic) or a pharmaceutically acceptable salt thereof:

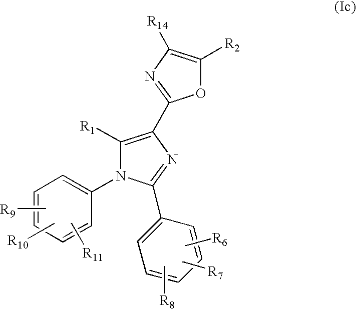

wherein R₁, R₆, R₇, R₈, R₉, R₁₀ and R₁₁ have the same meanings as defined above; and R₂ and R₁₄ are each independently hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₅, m being 1 or 2; or alternatively, R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

A still another embodiment of the present invention is to provide a compound of formula (Id) or a pharmaceutically acceptable salt thereof:

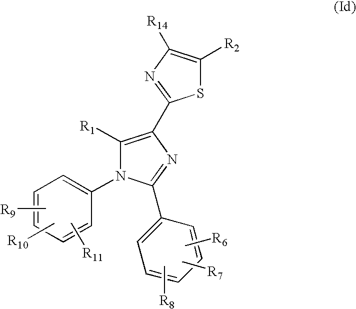

wherein, R₁, R₆, R₇, R₈, R₉, R₁₀ and R₁₁ have the same meanings as defined above; and R₂ and R₁₄ are each independently hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₅, m being 1 or 2, or alternatively, R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

Preferred compounds useful in the present invention are selected from the group consisting of:
2-tert-butyl-5-(1-(4-chlorophenyl)-2-(2,4-dichlorophenyl)-5-methyl-1H-imidazol-4-yl)-1,3,4-oxadiazole;
2-(1-(4-Chlorophenyl)-2-(2,4-dichlorophenyl)-5-methyl-1H-imidazol-4-yl)-5-cyclohexyl-1,3,4-oxadiazole;
2-(1-(4-Chlorophenyl)-2-(2,4-dichlorophenyl)-5-methyl-1H-imidazol-4-yl)-5-cyclopentyl-1,3,4-oxadiazole;
2-(1-(4-Chlorophenyl)-2-(2,4-dichlorophenyl)-5-methyl-1H-imidazol-4-yl)-5-cyclobutyl-1,3,4-oxadiazole;
2-(1-(4-Chlorophenyl)-2-(2,4-dichlorophenyl)-5-methyl-1H-imidazol-4-yl)-5-cycloheptyl-1,3,4-oxadiazole;
2-(1-(4-Chlorophenyl)-2-(2,4-dichlorophenyl)-5-methyl-1H-imidazol-4-yl)-5-(hexan-2-yl)-1,3,4-oxadiazole;
2-(1-(4-Chlorophenyl)-2-(2,4-dichlorophenyl)-5-methyl-1H-imidazol-4-yl)-5-(pentan-2-yl)-1,3,4-oxadiazole;
2-sec-Butyl-5-(1-(4-chlorophenyl)-2-(2,4-dichlorophenyl)-5-methyl-1H-imidazol-4-yl)-1,3,4-oxadiazole;
2-1-(4-Chlorophenyl)-2-(2,4-dichlorophenyl)-5-methyl-1H-imidazol-4-yl)-5-isopropyl-1,3,4-oxadiazole;
2-(1-(4-Chlorophenyl)-2-(2,4-dichlorophenyl)-5-methyl-1H-imidazol-4-yl)-5-(pentan-3-yl)-1,3,4-oxadiazole;
2-(1-(4-Chlorophenyl)-2-(2,4-dichlorophenyl)-5-methyl-1H-imidazol-4-yl)-5-(1-phenylethyl)-1,3,4-oxadiazole;
2-(1-(4-Chlorophenyl)-2-(2,4-dichlorophenyl)-5-ethyl-1H-imidazol-4-yl)-5-cyclohexyl-1,3,4-oxadiazole;
2-(1-(4-Chlorophenyl)-2-(2,4-dichlorophenyl)-5-ethyl-1H-imidazol-4-yl)-5-cyclopentyl-1,3,4-oxadiazole;
2-(1-(4-Chlorophenyl)-2-(2,4-dichlorophenyl)-5-ethyl-1H-imidazol-4-yl)-5-cyclobutyl-1,3,4-oxadiazole;
2-tert-butyl-5-(1-(4-chlorophenyl)-2-(2,4-dichlorophenyl)-5-ethyl-1H-imidazol-4-yl)-1,3,4-oxadiazole;
2-(1-(4-Chlorophenyl)-2-(2,4-dichlorophenyl)-5-ethyl-1H-imidazol-4-yl)-5-(hexan-2-yl)-1,3,4-oxadiazole;
2-(1-(4-Chlorophenyl)-2-(2,4-dichlorophenyl)-5-ethyl-1H-imidazol-4-yl)-5-(pentan-2-yl)-1,3,4-oxadiazole;
2-sec-Butyl-5-(1-(4-chlorophenyl)-2-(2,4-dichlorophenyl)-5-ethyl-1H-imidazol-4-yl)-1,3,4-oxadiazole;
2-(1-(4-Bromophenyl)-2-(2,4-dichlorophenyl)-5-methyl-1H-imidazol-4-yl)-5-cyclohexyl-1,3,4-oxadiazole;
2-(1-(4-Bromophenyl)-2-(2,4-dichlorophenyl)-5-methyl-1H-imidazol-4-yl)-5-tert-butyl-1,3,4-oxadiazole;
2-(1-(4-Bromophenyl)-2-(2,4-dichlorophenyl)-5-ethyl-1H-imidazol-4-yl)-5-tert-butyl-1,3,4-oxadiazole;
2-(1-(4-Bromophenyl)-2-(2,4-dichlorophenyl)-5-methyl-1H-imidazol-4-yl)-5-cyclobutyl-1,3,4-oxadiazole;
2-(1-(4-Bromophenyl)-2-(2,4-dichlorophenyl)-5-ethyl-1H-imidazol-4-yl)-5-cyclobutyl-1,3,4-oxadiazole;
2-tert-butyl-5-(1-(4-chlorophenyl)-5-cyclopropyl-2-(2,4-dichlorophenyl)-1H-imidazol-4-yl)-1,3,4-oxadiazole;
2-1-(4-Bromophenyl)-2-(2,4-dichlorophenyl)-5-isopropyl-1H-imidazol-4-yl)-5-tert-butyl-1,3,4-oxadiazole;
2-(1-(4-Bromophenyl)-2-(2,4-dichlorophenyl)-5-isopropyl-1H-imidazol-4-yl)-5-(1-phenylcyclopropyl)-1,3,4-oxadiazole;
2-(1-(4-Bromophenyl)-2-(2,4-dichlorophenyl)-5-ethyl-1H-imidazol-4-yl)-5-(1-phenylcyclopropyl)-1,3,4-oxadiazole;
2-(1-(4-Chlorophenyl)-2-(2,4-dichlorophenyl)-5-isopropyl-1H-imidazol-4-yl)-5-(1-phenylcyclopropyl)-1,3,4-oxadiazole;
2-(1-(4-Bromophenyl)-2-(2,4-dichlorophenyl)-5-propyl-1H-imidazol-4-yl)-5-tert-butyl-1,3,4-oxadiazole;
2-(1-(4-Bromophenyl)-2-(2,4-dichlorophenyl)-5-propyl-1H-imidazol-4-yl)-5-cyclohexyl-1,3,4-oxadiazole;
2-(1-(4-Bromophenyl)-2-(2,4-dichlorophenyl)-5-propyl-1H-imidazol-4-yl)-5-(1-phenylcyclopropyl)-1,3,4-oxadiazole;
2-(1-(4-Bromophenyl)-2-(2,4-dichlorophenyl)-5-propyl-1H-imidazol-4-yl)-5-cyclobutyl-1,3,4-oxadiazole;
2-(1-(4-Bromophenyl)-2-(2,4-dichlorophenyl)-5-propyl-1H-imidazol-4-yl)-5-(1-(4-chlorophenyl)cyclopropyl)-1,3,4-oxadiazole;
2-(1-(4-Bromophenyl)-2-(2,4-dichlorophenyl)-5-propyl-1H-imidazol-4-yl)-5-(1-(4-chlorophenyl)cyclobutyl)-1,3,4-oxadiazole;
2-tert-butyl-5-(1-(4-chlorophenyl)-2-(2,4-dichlorophenyl)-5-propyl-1H-imidazol-4-yl)-1,3,4-oxadiazole;
2-(1-(4-Chlorophenyl)-2-(2,4-dichlorophenyl)-5-propyl-1H-imidazol-4-yl)-5-cyclobutyl-1,3,4-oxadiazole;
2-(1-(4-Chlorophenyl)-2-(2,4-dichlorophenyl)-5-propyl-1H-imidazol-4-yl)-5-(1-(4-chlorophenyl)cyclopropyl)-1,3,4-oxadiazole;
2-(1-(4-Chlorophenyl)-2-(2,4-dichlorophenyl)-5-propyl-1H-imidazol-4-yl)-5-(1-(4-chlorophenyl)cyclobutyl)-1,3,4-oxadiazole;
2-(1-(4-Chlorophenyl)-2-(2,4-dichlorophenyl)-5-propyl-1H-imidazol-4-yl)-5-cyclohexyl-1,3,4-oxadiazole;
2-(1-(4-Bromophenyl)-2-(2,4-dichlorophenyl)-5-ethyl-1H-imidazol-4-yl)-5-(1-(4-chlorophenyl)cyclopropyl)-1,3,4-oxadiazole;
2-(1-(4-Bromophenyl)-2-(2,4-dichlorophenyl)-5-ethyl-1H-imidazol-4-yl)-5-(1-(4-chlorophenyl)cyclobutyl)-1,3,4-oxadiazole;
2-(1-(4-Bromophenyl)-2-(2,4-dichlorophenyl)-5-ethyl-1H-imidazol-4-yl)-5-(1-(4-methoxyphenyl)cyclopropyl)-1,3,4-oxadiazole;
2-(1-(4-Bromophenyl)-2-(2,4-dichlorophenyl)-5-ethyl-1H-imidazol-4-yl)-5-(1-p-tolylcyclopropyl)-1,3,4-oxadiazole;
2-(1-(4-Bromophenyl)-2-(2,4-dichlorophenyl)-5-ethyl-1H-imidazol-4-yl)-5-(1-(2,4-dichlorophenyl)cyclopropyl)-1,3,4-oxadiazole;
2-(1-(4-Bromophenyl)-2-(2,4-dichlorophenyl)-5-ethyl-1H-imidazol-4-yl)-5-(1-(4-chlorophenyl)cyclopentyl)-1,3,4-oxadiazole;
2-(1-(4-Bromophenyl)-2-(2,4-dichlorophenyl)-5-methyl-1H-imidazol-4-yl)-5-(1-(trifluoromethyl)cyclopropyl)-1,3,4-oxadiazole;
2-(1-(4-Bromophenyl)-2-(2,4-dichlorophenyl)-5-methyl-1H-imidazol-4-yl)-5-(1-(trifluoromethyl)cyclobutyl)-1,3,4-oxadiazole;
2-(1-(4-Bromophenyl)-2-(2,4-dichlorophenyl)-5-methyl-1H-imidazol-4-yl)-5-(1-(4-chlorophenyl)cyclopropyl)-1,3,4-oxadiazole;
2-(1-(4-Bromophenyl)-2-(2,4-dichlorophenyl)-5-ethyl-1H-imidazol-4-yl)-5-(1-(trifluoromethyl)cyclopropyl)-1,3,4-oxadiazole;
2-(1-(4-Bromophenyl)-2-(2,4-dichlorophenyl)-5-ethyl-1H-imidazol-4-yl)-5-(1-(trifluoromethyl)cyclobutyl)-1,3,4-oxadiazole;
2-(1-(4-Bromophenyl)-2-(2-chlorophenyl)-5-methyl-1H-imidazol-4-yl)-5-tert-butyl-1,3,4-oxadiazole;
2-(1-(4-Bromophenyl)-2-(2-chlorophenyl)-5-methyl-1H-imidazol-4-yl)-5-(1-(trifluoromethyl)cyclopropyl)-1,3,4-oxadiazole;
2-(1-(4-Bromophenyl)-2-(2-chlorophenyl)-5-methyl-1H-imidazol-4-yl)-5-(1-(trifluoromethyl)cyclobutyl)-1,3,4-oxadiazole;
2-(1-(4-Bromophenyl)-2-(2-chlorophenyl)-5-methyl-1H-imidazol-4-yl)-5-(1-(4-chlorophenyl)cyclopropyl)-1,3,4-oxadiazole;
2-(1-(4-Bromophenyl)-2-(2-chlorophenyl)-5-ethyl-1H-imidazol-4-yl)-5-tert-butyl-1,3,4-oxadiazole;
2-(1-(4-Bromophenyl)-2-(2-chlorophenyl)-5-ethyl-1H-imidazol-4-yl)-5-(1-(trifluoromethyl)cyclopropyl)-1,3,4-oxadiazole;
2-(1-(4-Bromophenyl)-2-(2-chlorophenyl)-5-ethyl-1H-imidazol-4-yl)-5-(1-(trifluoromethyl)cyclobutyl)-1,3,4-oxadiazole;
2-(1-(4-Bromophenyl)-2-(2-chlorophenyl)-5-ethyl-1H-imidazol-4-yl)-5-(1-(4-chlorophenyl)cyclopropyl)-1,3,4-oxadiazole;
2-tert-Butyl-5-(2-(2-chlorophenyl)-1-(4-chlorophenyl)-5-methyl-1H-imidazol-4-yl)-1,3,4-oxadiazole;
2-(2-(2-Chlorophenyl)-1-(4-chlorophenyl)-5-methyl-1H-imidazol-4-yl)-5-(1-(trifluoromethyl)cyclopropyl)-1,3,4-oxadiazole;
2-(2-(2-Chlorophenyl)-1-(4-chlorophenyl)-5-methyl-1H-imidazol-4-yl)-5-(1-(trifluoromethyl)cyclobutyl)-1,3,4-oxadiazole;
2-(2-(2-Chlorophenyl)-1-(4-chlorophenyl)-5-methyl-1H-imidazol-4-yl)-5-(1-(4-chlorophenyl)cyclopropyl)-1,3,4-oxadiazole;
2-tert-Butyl-5-(2-(2-chlorophenyl)-1-(4-chlorophenyl)-5-ethyl-1H-imidazol-4-yl)-1,3,4-oxadiazole;
2-(2-(2-Chlorophenyl)-1-(4-chlorophenyl)-5-ethyl-1H-imidazol-4-yl)-5-(1-(trifluoromethyl)cyclopropyl)-1,3,4-oxadiazole;
2-(2-(2-Chlorophenyl)-1-(4-chlorophenyl)-5-ethyl-1H-imidazol-4-yl)-5-(1-(trifluoromethyl)cyclobutyl)-1,3,4-oxadiazole;

2-(2-(2-Chlorophenyl)-1-(4-chlorophenyl)-5-ethyl-1H-imidazol-4-yl)-5-(1-(4-chlorophenyl)cyclopropyl)-1,3,4-oxadiazole;
2-(1-(4-Chlorophenyl)-2-(2,4-dichlorophenyl)-5-methyl-1H-imidazol-4-yl)-5-(1-(trifluoromethyl)cyclopropyl)-1,3,4-oxadiazole;
2-(1-(4-Chlorophenyl)-2-(2,4-dichlorophenyl)-5-methyl-1H-imidazol-4-yl)-5-(1-(trifluoromethyl)cyclobutyl)-1,3,4-oxadiazole;
2-(1-(4-Chlorophenyl)-2-(2,4-dichlorophenyl)-5-methyl-1H-imidazol-4-yl)-5-(1-(4-chlorophenyl)cyclopropyl)-1,3,4-oxadiazole;
2-(1-(4-Chlorophenyl)-2-(2,4-dichlorophenyl)-5-ethyl-1H-imidazol-4-yl)-5-(1-(trifluoromethyl)cyclopropyl)-1,3,4-oxadiazole;
2-(1-(4-Chlorophenyl)-2-(2,4-dichlorophenyl)-5-ethyl-1H-imidazol-4-yl)-5-(1-(trifluoromethyl)cyclobutyl)-1,3,4-oxadiazole;
2-(1-(4-Chlorophenyl)-2-(2,4-dichlorophenyl)-5-ethyl-1H-imidazol-4-yl)-5-(1-(4-chlorophenyl)cyclopropyl)-1,3,4-oxadiazole;
2-(1-(4-Chlorophenyl)-2-(2,4-dichlorophenyl)-5-ethyl-1H-imidazol-4-yl)-5-(trifluoromethyl)-1,3,4-oxadiazole;
2-(5-((1H-1,2,4-Triazol-1-yl)methyl)-1-(4-bromophenyl)-2-(2,4-dichlorophenyl)-1H-imidazol-4-yl)-5-tert-butyl-1,3,4-oxadiazole;
2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclopropyl)-1,3,4-oxadiazole;
2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclobutyl)-1,3,4-oxadiazole;
2-(5-((1H-1,2,4-Triazol-1-yl)methyl)-1-(4-bromophenyl)-2-(2,4-dichlorophenyl)-1H-imidazol-4-yl)-5-(1-(4-chlorophenyl)cyclopropyl)-1,3,4-oxadiazole;
2-(5-((1H-1,2,4-Triazol-1-yl)methyl)-1-(4-bromophenyl)-2-(2-chlorophenyl)-1H-imidazol-4-yl)-5-tert-butyl-1,3,4-oxadiazole;
2-(5-((1H-1,2,4-Triazol-1-yl)methyl)-1-(4-bromophenyl)-2-(2-chlorophenyl)-1H-imidazol-4-yl)-5-(1-(trifluoromethyl)cyclopropyl)-1,3,4-oxadiazole;
2-(5-((1H-1,2,4-Triazol-1-yl)methyl)-1-(4-bromophenyl)-2-(2-chlorophenyl)-1H-imidazol-4-yl)-5-(1-(trifluoromethyl)cyclobutyl)-1,3,4-oxadiazole;
2-(5-((1H-1,2,4-Triazol-1-yl)methyl)-1-(4-bromophenyl)-2-(2-chlorophenyl)-1H-imidazol-4-yl)-5-(1-(4-chlorophenyl)cyclopropyl)-1,3,4-oxadiazole;
2-(5-((1H-1,2,4-Triazol-1-yl)methyl)-2-(2-chlorophenyl)-1-(4-chlorophenyl)-1H-imidazol-4-yl)-5-tert-butyl-1,3,4-oxadiazole;
2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclopropyl)-1,3,4-oxadiazole;
2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclobutyl)-1,3,4-oxadiazole;
2-(5-((1H-1,2,4-Triazol-1-yl)methyl)-2-(2-chlorophenyl)-1-(4-chlorophenyl)-1H-imidazol-4-yl)-5-(1-(4-chlorophenyl)cyclopropyl)-1,3,4-oxadiazole;
2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclobutyl)-1,3,4-oxadiazole;
2-(5-((1H-1,2,4-Triazol-1-yl)methyl)-1-(4-chlorophenyl)-2-(2,4-dichlorophenyl)-1H-imidazol-4-yl)-5-tert-butyl-1,3,4-oxadiazole;
2-(5-((1H-1,2,4-Triazol-1-yl)methyl)-1-(4-chlorophenyl)-2-(2,4-dichlorophenyl)-1H-imidazol-4-yl)-5-(1-(4-chlorophenyl)cyclopropyl)-1,3,4-oxadiazole;
2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclopropyl)-1,3,4-oxadiazole;
2-tert-butyl-5-(1-(4-chlorophenyl)-2-(2,4-dichlorophenyl)-5-methyl-1H-imidazol-4-yl)-1,3,4-thiadiazole;
2-(1-(4-Chlorophenyl)-2-(2,4-dichlorophenyl)-5-methyl-1H-imidazol-4-yl)-5-cyclohexyl-1,3,4-thiadiazole;
2-(1-(4-Chlorophenyl)-2-(2,4-dichlorophenyl)-5-methyl-1H-imidazol-4-yl)-5-cyclopentyl-1,3,4-thiadiazole;
2-(1-(4-Chlorophenyl)-2-(2,4-dichlorophenyl)-5-methyl-1H-imidazol-4-yl)-5-cyclobutyl-1,3,4-thiadiazole;
2-(1-(4-Chlorophenyl)-2-(2,4-dichlorophenyl)-5-methyl-1H-imidazol-4-yl)-5-cycloheptyl-1,3,4-thiadiazole;
2-(1-(4-Chlorophenyl)-2-(2,4-dichlorophenyl)-5-methyl-1H-imidazol-4-yl)-5-(hexan-2-yl)-1,3,4-thiadiazole;
2-(1-(4-Chlorophenyl)-2-(2,4-dichlorophenyl)-5-methyl-1H-imidazol-4-yl)-5-(pentan-2-yl)-1,3,4-thiadiazole;
2-sec-Butyl-5-(1-(4-chlorophenyl)-2-(2,4-dichlorophenyl)-5-methyl-1H-imidazol-4-yl)-1,3,4-thiadiazole;
2-(1-(4-Chlorophenyl)-2-(2,4-dichlorophenyl)-5-methyl-1H-imidazol-4-yl)-5-isopropyl-1,3,4-thiadiazole;
2-(1-(4-Chlorophenyl)-2-(2,4-dichlorophenyl)-5-methyl-1H-imidazol-4-yl)-5-(pentan-3-yl)-1,3,4-thiadiazole;
2-(1-(4-Chlorophenyl)-2-(2,4-dichlorophenyl)-5-methyl-1H-imidazol-4-yl)-5-(1-phenylethyl)-1,3,4-thiadiazole;
2-(1-(4-Chlorophenyl)-2-(2,4-dichlorophenyl)-5-ethyl-1H-imidazol-4-yl)-5-cyclohexyl-1,3,4-thiadiazole;
2-(1-(4-Chlorophenyl)-2-(2,4-dichlorophenyl)-5-ethyl-1H-imidazol-4-yl)-5-cyclopentyl-1,3,4-thiadiazole;
2-(1-(4-Chlorophenyl)-2-(2,4-dichlorophenyl)-5-ethyl-1H-imidazol-4-yl)-5-cyclobutyl-1,3,4-thiadiazole;
2-tert-Butyl-5-(1-(4-chlorophenyl)-2-(2,4-dichlorophenyl)-5-ethyl-1H-imidazol-4-yl)-1,3,4-thiadiazole;
2-(1-(4-Chlorophenyl)-2-(2,4-dichlorophenyl)-5-ethyl-1H-imidazol-4-yl)-5-(hexan-2-yl)-1,3,4-thiadiazole;
2-(1-(4-Chlorophenyl)-2-(2,4-dichlorophenyl)-5-ethyl-1H-imidazol-4-yl)-5-(pentan-2-yl)-1,3,4-thiadiazole;
2-sec-Butyl-5-(1-(4-chlorophenyl)-2-(2,4-dichlorophenyl)-5-ethyl-1H-imidazol-4-yl)-1,3,4-thiadiazole;
2-(1-(4-Bromophenyl)-2-(2,4-dichlorophenyl)-5-methyl-1H-imidazol-4-yl)-5-cyclohexyl-1,3,4-thiadiazole;
2-(1-(4-Bromophenyl)-2-(2,4-dichlorophenyl)-5-methyl-1H-imidazol-4-yl)-5-tert-butyl-1,3,4-thiadiazole;
2-(1-(4-Bromophenyl)-2-(2,4-dichlorophenyl)-5-ethyl-1H-imidazol-4-yl)-5-tert-butyl-1,3,4-thiadiazole;
2-(1-(4-Bromophenyl)-2-(2,4-dichlorophenyl)-5-methyl-1H-imidazol-4-yl)-5-cyclobutyl-1,3,4-thiadiazole;
2-(1-(4-Bromophenyl)-2-(2,4-dichlorophenyl)-5-ethyl-1H-imidazol-4-yl)-5-cyclobutyl-1,3,4-thiadiazole;
2-tert-Butyl-5-(1-(4-chlorophenyl)-5-cyclopropyl-2-(2,4-dichlorophenyl)-1H-imidazol-4-yl)-1,3,4-thiadiazole;
2-(1-(4-Bromophenyl)-2-(2,4-dichlorophenyl)-5-isopropyl-1H-imidazol-4-yl)-5-tert-butyl-1,3,4-thiadiazole;
2-(1-(4-Bromophenyl)-2-(2,4-dichlorophenyl)-5-isopropyl-1H-imidazol-4-yl)-5-cyclobutyl-1,3,4-thiadiazole;
2-(1-(4-Bromophenyl)-2-(2,4-dichlorophenyl)-5-isopropyl-1H-imidazol-4-yl)-5-(1-phenylcyclopropyl)-1,3,4-thiadiazole;
2-(1-(4-Bromophenyl)-2-(2,4-dichlorophenyl)-5-ethyl-1H-imidazol-4-yl)-5-(1-phenylcyclopropyl)-1,3,4-thiadiazole;
2-(1-(4-Chlorophenyl)-2-(2,4-dichlorophenyl)-5-isopropyl-1H-imidazol-4-yl)-5-(1-phenylcyclopropyl)-1,3,4-thiadiazole;
2-(1-(4-Bromophenyl)-2-(2,4-dichlorophenyl)-5-propyl-1H-imidazol-4-yl)-5-tert-butyl-1,3,4-thiadiazole;
2-(1-(4-Bromophenyl)-2-(2,4-dichlorophenyl)-5-propyl-1H-imidazol-4-yl)-5-cyclohexyl-1,3,4-thiadiazole;
2-(1-(4-Bromophenyl)-2-(2,4-dichlorophenyl)-5-propyl-1H-imidazol-4-yl)-5-(1-phenylcyclopropyl)-1,3,4-thiadiazole;
2-(1-(4-Bromophenyl)-2-(2,4-dichlorophenyl)-5-propyl-1H-imidazol-4-yl)-5-cyclobutyl-1,3,4-thiadiazole;
2-(1-(4-Bromophenyl)-2-(2,4-dichlorophenyl)-5-propyl-1H-imidazol-4-yl)-5-(1-(4-chlorophenyl)cyclopropyl)-1,3,4-thiadiazole;
2-(1-(4-Bromophenyl)-2-(2,4-dichlorophenyl)-5-propyl-1H-imidazol-4-yl)-5-(1-(4-chlorophenyl)cyclobutyl)-1,3,4-thiadiazole;
2-tert-Butyl-5-(1-(4-chlorophenyl)-2-(2,4-dichlorophenyl)-5-propyl-1H-imidazol-4-yl)-1,3,4-thiadiazole;
2-(1-(4-Chlorophenyl)-2-(2,4-dichlorophenyl)-5-propyl-1H-imidazol-4-yl)-5-cyclobutyl-1,3,4-thiadiazole;
2-(1-(4-Chlorophenyl)-2-(2,4-dichlorophenyl)-5-propyl-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-propyl-1H-imidazol-4-yl)-5-(1-(4-chlorophenyl)cyclobutyl)-1,3,4-thiadiazole;
b 2-(1-(4-Chlorophenyl)-2-(2,4-dichlorophenyl)-5-propyl-1H-imidazol-4-yl)-5-cyclohexyl-1,3,4-thiadiazole;
2-(1-(4-Bromophenyl)-2-(2,4-dichlorophenyl)-5-ethyl-1H-imidazol-4-yl)-5-(1-(4-chlorophenyl)cyclopropyl)-1,3,4-thiadiazole;
2-(1-(4-Bromophenyl)-2-(2,4-dichlorophenyl)-5-ethyl-1H-imidazol-4-yl)-5-(1-(4-chlorophenyl)cyclobutyl)-1,3,4-thiadiazole;
2-(1-(4-Bromophenyl)-2-(2,4-dichlorophenyl)-5-ethyl-1H-imidazol-4-yl)-5-(1-(4-methoxyphenyl)cyclopropyl)-1,3,4-thiadiazole;
2-(1-(4-Bromophenyl)-2-(2,4-dichlorophenyl)-5-ethyl-1H-imidazol-4-yl)-5-(1-p-tolylcyclopropyl)-1,3,4-thiadiazole;
2-(1-(4-Bromophenyl)-2-(2,4-dichlorophenyl)-5-ethyl-1H-imidazol-4-yl)-5-(1-(2,4-dichlorophenyl)cyclopropyl)-1,3,4-thiadiazole;
2-(1-(4-Bromophenyl)-2-(2,4-dichlorophenyl)-5-ethyl-1H-imidazol-4-yl)-5-(1-(4-chlorophenyl)cyclopentyl)-1,3,4-thiadiazole;
2-(1-(4-Bromophenyl)-2-(2,4-dichlorophenyl)-5-methyl-1H-imidazol-4-yl)-5-(1-(trifluoromethyl)cyclopropyl)-1,3,4-thiadiazole;
2-(1-(4-Bromophenyl)-2-(2,4-dichlorophenyl)-5-methyl-1H-imidazol-4-yl)-5-(1-(trifluoromethyl)cyclobutyl)-1,3,4-thiadiazole;
2-(1-(4-Bromophenyl)-2-(2,4-dichlorophenyl)-5-methyl-1H-imidazol-4-yl)-5-(1-(4-chlorophenyl)cyclopropyl)-1,3,4-thiadiazole;
2-(1-(4-Bromophenyl)-2-(2,4-dichlorophenyl)-5-ethyl-1H-imidazol-4-yl)-5-(1-(trifluoromethyl)cyclopropyl)-1,3,4-thiadiazole;
2-(1-(4-Bromophenyl)-2-(2,4-dichlorophenyl)-5-ethyl-1H-imidazol-4-yl)-5-(1-(trifluoromethyl)cyclobutyl)-1,3,4-thiadiazole;
2-(1-(4-Bromophenyl)-2-(2-chlorophenyl)-5-methyl-1H-imidazol-4-yl)-5-tert-butyl-1,3,4-thiadiazole;
2-(1-(4-Bromophenyl)-2-(2-chlorophenyl)-5-methyl-1H-imidazol-4-yl)-5-(1-(trifluoromethyl)cyclopropyl)-1,3,4-thiadiazole;
2-(1-(4-Bromophenyl)-2-(2-chlorophenyl)-5-methyl-1H-imidazol-4-yl)-5-(1-(trifluoromethyl)cyclobutyl)-1,3,4-thiadiazole;
2-(1-(4-Bromophenyl)-2-(2-chlorophenyl)-5-methyl-1H-imidazol-4-yl)-5-(1-(4-chlorophenyl)cyclopropyl)-1,3,4-thiadiazole;
2-(1-(4-Bromophenyl)-2-(2-chlorophenyl)-5-ethyl-1H-imidazol-4-yl)-5-tert-butyl-1,3,4-thiadiazole;
2-(1-(4-Bromophenyl)-2-(2-chlorophenyl)-5-ethyl-1H-imidazol-4-yl)-5-(1-(trifluoromethyl)cyclopropyl)-1,3,4-thiadiazole;
2-(1-(4-Bromophenyl)-2-(2-chlorophenyl)-5-ethyl-1H-imidazol-4-yl)-5-(1-(trifluoromethyl)cyclobutyl)-1,3,4-thiadiazole;
2-(1-(4-Bromophenyl)-2-(2-chlorophenyl)-5-ethyl-1H-imidazol-4-yl)-5-(1-(4-chlorophenyl)cyclopropyl)-1,3,4-thiadiazole;
2-tert-Butyl-5-(2-(2-chlorophenyl)-1-(4-chlorophenyl)-5-methyl-1H-imidazol-4-yl)-1,3,4-thiadiazole;
2-(2-(2-Chlorophenyl)-1-(4-chlorophenyl)-5-methyl-1H-imidazol-4-yl)-5-(1-(trifluoromethyl)cyclopropyl)-1,3,4-thiadiazole;
2-(2-(2-Chlorophenyl)-1-(4-chlorophenyl)-5-methyl-1H-imidazol-4-yl)-5-(1-(trifluoromethyl)cyclobutyl)-1,3,4-thiadiazole;
2-(2-(2-Chlorophenyl)-1-(4-chlorophenyl)-5-methyl-1H-imidazol-4-yl)-5-(1-(4-chlorophenyl)cyclopropyl)-1,3,4-thiadiazole;
2-tert-Butyl-5-(2-(2-chlorophenyl)-1-(4-chlorophenyl)-5-ethyl-1H-imidazol-4-yl)-1,3,4-thiadiazole;
2-(2-(2-Chlorophenyl)-1-(4-chlorophenyl)-5-ethyl-1H-imidazol-4-yl)-5-(1-(trifluoromethyl)cyclopropyl)-1,3,4-thiadiazole;
2-(2-(2-Chlorophenyl)-1-(4-chlorophenyl)-5-ethyl-1H-imidazol-4-yl)-5-(1-(trifluoromethyl)cyclobutyl)-1,3,4-thiadiazole;
2-(2-(2-Chlorophenyl)-1-(4-chlorophenyl)-5-ethyl-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-methyl-1H-imidazol-4-yl)-5-(1-(trifluoromethyl)cyclopropyl)-1,3,4-thiadiazole;
2-(1-(4-Chlorophenyl)-2-(2,4-dichlorophenyl)-5-methyl-1H-imidazol-4-yl)-5-(1-(trifluoromethyl)cyclobutyl)-1,3,4-thiadiazole;
2-(1-(4-Chlorophenyl)-2-(2,4-dichlorophenyl)-5-methyl-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-ethyl-1H-imidazol-4-yl)-5-(1-(trifluoromethyl)cyclopropyl)-1,3,4-thiadiazole;
2-(1-(4-Chlorophenyl)-2-(2,4-dichlorophenyl)-5-ethyl-1H-imidazol-4-yl)-5-(1-(trifluoromethyl)cyclobutyl)-1,3,4-thiadiazole;
2-(1-(4-Chlorophenyl)-2-(2,4-dichlorophenyl)-5-ethyl-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-ethyl-1H-imidazol-4-yl)-5-(trifluoromethyl)-1,3,4-thiadiazole;
2-(5-((1H-1,2,4-Triazol-1-yl)methyl)-1-(4-bromophenyl)-2-(2,4-dichlorophenyl)-1H-imidazol-4-yl)-5-tert-butyl-1,3,4-thiadiazole;
2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclopropyl)-1,3,4-thiadiazole;
2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclobutyl)-1,3,4-thiadiazole;
2-(5-((1H-1,2,4-Triazol-1-yl)methyl)-1-(4-bromophenyl)-2-(2,4-dichlorophenyl)-1H-imidazol-4-yl)-5-(1-(4-chlorophenyl)cyclopropyl)-1,3,4-thiadiazole;
2-(5-((1H-1,2,4-Triazol-1-yl)methyl)-1-(4-bromophenyl)-2-(2-chlorophenyl)-1H-imidazol-4-yl)-5-tert-butyl-1,3,4-thiadiazole;
2-(5-((1H-1,2,4-Triazol-1-yl)methyl)-1-(4-bromophenyl)-2-(2-chlorophenyl)-1H-imidazol-4-yl)-5-(1-(trifluoromethyl)cyclopropyl)-1,3,4-thiadiazole;
2-(5-((1H-1,2,4-Triazol-1-yl)methyl)-1-(4-bromophenyl)-2-(2-chlorophenyl)-1H-imidazol-4-yl)-5-(1-(trifluoromethyl)cyclobutyl)-1,3,4-thiadiazole;
2-(5-((1H-1,2,4-Triazol-1-yl)methyl)-1-(4-bromophenyl)-2-(2-chlorophenyl)-1H-imidazol-4-yl)-5-(1-(4-chlorophenyl)cyclopropyl)-1,3,4-thiadiazole;
2-(5-((1H-1,2,4-Triazol-1-yl)methyl)-2-(2-chlorophenyl)-1-(4-chlorophenyl)-1H-imidazol-4-yl)-5-tert-butyl-1,3,4-thiadiazole;
2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclopropyl)-1,3,4-thiadiazole;
2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclobutyl)-1,3,4-thiadiazole;
2-(5-((1H-1,2,4-Triazol-1-yl)methyl)-2-(2-chlorophenyl)-1-(4-chlorophenyl)-1H-imidazol-4-yl)-5-(1-(4-chlorophenyl)cyclopropyl)-1,3,4-thiadiazole;
2-(5-((1H-1,2,4-Triazol-1-yl)methyl)-1-(4-chlorophenyl)-2-(2,4-dichlorophenyl)-1H-imidazol-4-yl)-5-tert-butyl-1,3,4-thiadiazole;
2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclopropyl)-1,3,4-thiadiazole;
2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclobutyl)-1,3,4-thiadiazole;
2-(5-((1H-1,2,4-Triazol-1-yl)methyl)-1-(4-chlorophenyl)-2-(2,4-dichlorophenyl)-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-methyl-1H-imidazol-4-yl)-5-cyclohexyloxazole;
2-(1-(4-Chlorophenyl)-2-(2,4-dichlorophenyl)-5-methyl-1H-imidazol-4-yl)-5-ethyloxazole;
5-Butyl-2-(1-(4-chlorophenyl)-2-(2,4-dichlorophenyl)-5-methyl-1H-imidazol-4-yl)oxazole;
2-(1-(4-Chlorophenyl)-2-(2,4-dichlorophenyl)-5-methyl-1H-imidazol-4-yl)-5-isopropyloxazole;
2-(1-(4-Chlorophenyl)-2-(2,4-dichlorophenyl)-5-methyl-1H-imidazol-4-yl)-5-isobutyloxazole;
2-tert-Butyl-5-(1-(4-chlorophenyl)-2-(2,4-dichlorophenyl)-5-methyl-1H-imidazol-4-yl)oxazole;
2-tert-Butyl-5-(1-(4-chlorophenyl)-2-(2,4-dichlorophenyl)-5-ethyl-1H-imidazol-4-yl)oxazole;
2-(1-(4-Bromophenyl)-2-(2,4-dichlorophenyl)-5-methyl-1H-imidazol-4-yl)-5-tert-butyloxazole;
2-(1-(4-Bromophenyl)-2-(2,4-dichlorophenyl)-5-ethyl-1H-imidazol-4-yl)-5-tert-butyloxazole;
4-tert-Butyl-2-(1-(4-chlorophenyl)-2-(2,4-dichlorophenyl)-5-methyl-1H-imidazol-4-yl)oxazole;
4-tert-Butyl-2-(1-(4-chlorophenyl)-2-(2,4-dichlorophenyl)-5-ethyl-1H-imidazol-4-yl)oxazole;
2-(1-(4-Bromophenyl)-2-(2,4-dichlorophenyl)-5-ethyl-1H-imidazol-4-yl)-4-tert-butyloxazole;
2-(1-(4-Chlorophenyl)-2-(2,4-dichlorophenyl)-5-methyl-1H-imidazol-4-yl)-5-ethyl-4-methyloxazole;
2-(1-(4-Chlorophenyl)-2-(2,4-dichlorophenyl)-5-methyl-1H-imidazol-4-yl)-5-cyclohexylthiazole;
2-(1-(4-Chlorophenyl)-2-(2,4-dichlorophenyl)-5-methyl-1H-imidazol-4-yl)-5-ethylthiazole;
5-Butyl-2-(1-(4-chlorophenyl)-2-(2,4-dichlorophenyl)-5-methyl-1H-imidazol-4-yl)thiazole;
2-(1-(4-Chlorophenyl)-2-(2,4-dichlorophenyl)-5-methyl-1H-imidazol-4-yl)-5-isopropylthiazole;
2-(1-(4-Chlorophenyl)-2-(2,4-dichlorophenyl)-5-methyl-1H-imidazol-4-yl)-5-isobutylthiazole;
2-tert-Butyl-5-(1-(4-chlorophenyl)-2-(2,4-dichlorophenyl)-5-methyl-1H-imidazol-4-yl)thiazole;
2-tert-Butyl-5-(1-(4-chlorophenyl)-2-(2,4-dichlorophenyl)-5-ethyl-1H-imidazol-4-yl)thiazole;
2-(1-(4-Bromophenyl)-2-(2,4-dichlorophenyl)-5-methyl-1H-imidazol-4-yl)-5-tert-butylthiazole;
2-(1-(4-Bromophenyl)-2-(2,4-dichlorophenyl)-5-ethyl-1H-imidazol-4-yl)-5-tert-butylthiazole; and
2-(1-(4-Chlorophenyl)-2-(2,4-dichlorophenyl)-5-methyl-1H-imidazol-4-yl)-5-ethyl-4-methylthiazole.

It is to be understood that the present invention also includes a pharmaceutically acceptable salt and an addition salt of the inventive compound, such as a hydrochloride, hydrobromide or trifluoroacetate addition salt and a sodium, potassium and magnesium salt.

The compounds of the present invention may contain one or more asymmetric carbon atoms and may exist in racemic and optically active forms. All of these compounds and diastereomers are incorporated within the scope of the present invention.

The compounds and processes of the present invention will be better understood in connection with the following synthetic schemes, which are merely illustrative of the methods by which the compounds of the invention may be prepared and are not intended to limit the scope of the invention as defined in the appended claims.

The compound of formula (Ia) may be prepared by (i) reacting a carboxylic acid derivative (5) with a hydrazide compound (7) in the presence of a coupling agent, e.g. EDCI, DMAP, and (ii) cyclizing the resulting product (9) using a dehydrating agent to obtain an 1,3,4-oxadiazole compound, as shown in Reaction Scheme 1. The cyclization may be conducted using Burgess reagent as a dehydrating agent while applying microwave irradiation thereon (See Leber, J. D. et al., WO 2005/032550), or using triphenylphosphine with carbon tetrachloride and a base such as triethylamine in a suitable solvent such as acetonitrile and THF, or using POCl₃ in an appropriate solvent such as acetonitrile.

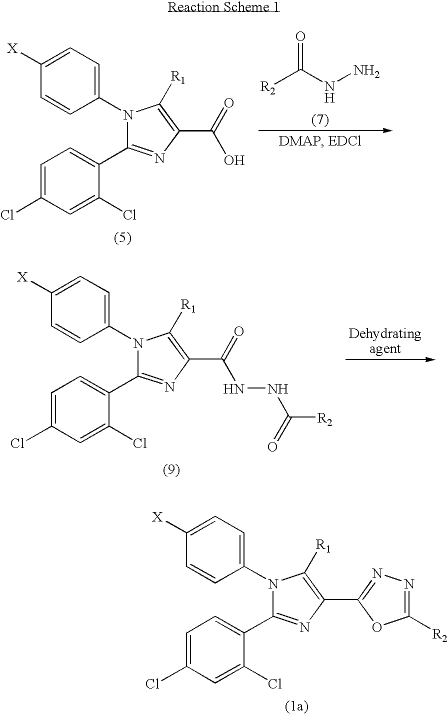

wherein, R₁ and R₂ have the same meanings as defined above, and X is halogen.

The carboxylic acid derivative (5) used as a starting material in preparing the compound of formula (Ia) may be prepared by a conventional method, e.g., by reacting a benzonitrile derivative (1) with an aniline derivative such as 4-chloroaniline using a non-nucleophilic base such as sodium bis(trimethylsilyl)amide (NaHMDS) to produce a corresponding arylbenzamidine (2). Subsequent reaction of the resulting arylbenzamidine (2) with ethyl 3-bromo-2-oxobutanoate (3) to provide an intermediate ethyl 1,2-diaryl-5-methyl-1H-imidazole-4-carboxylate (4). Transforming the intermediate (4) into an acid form (5) using an alkaline agent such as potassium hydroxide or lithium hydroxide, followed by acidification (See Lange, J. H. M. et al., J. Med. Chem. 2005, 48, 1823), as shown in Reaction Scheme 2.

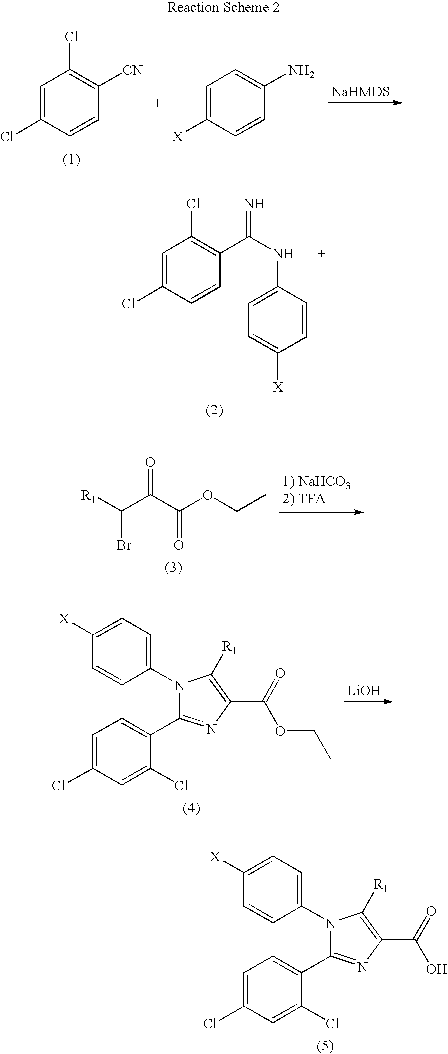

wherein, R₁ has the same meaning as defined above and X is halogen.

The hydrazide compound (7) which may be used in preparing the compound of formula (la) may be prepared by treating an ester or a carboxylic acid with hydrazine, as shown in Reaction Scheme 3.

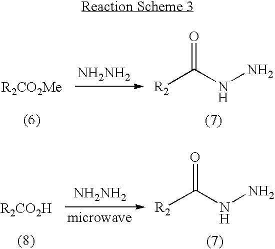

wherein, R₂ has the same meaning as defined above.

The compound of formula (Ib) may be prepared by (i) reacting a carboxylic acid derivative (5) with a hydrazide compound (7) in the presence of coupling reagents, e.g., EDCI, DMAP, and (ii) cyclizing the resulting product using a Lawesson's reagent, which can be conducted with microwave irradiation (See Kiryanov, A. A., Sampson, P., Seed, A. J., J. Org. Chem. 2001, 665, 7925-7929), as shown in Reaction Scheme 4.

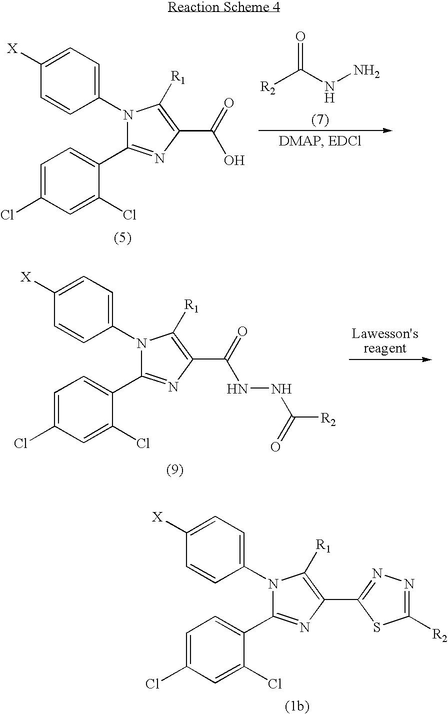

wherein, R₁ and R₂ have the same meanings as defined above.

The compound of formula (Ic) may be prepared by (i) reacting a carboxylic acid intermediate (5) with an aminoketone (10) in the presence of a coupling reagent, e. g. EDCI, DMAP, and (ii) cyclizing the resulting product using a dehydrating agent to obtain an oxazole compound of formula (Ic), as shown in Reaction Scheme 5.

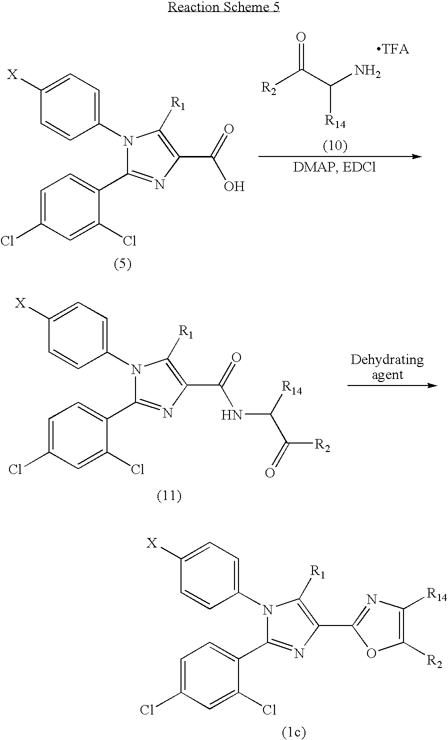

wherein R₁, R₂ and R₁₄ have the same meanings as defined above.

The aminoketone compound which may be used in preparing the compound of formula (Ic) may be prepared as shown in Reaction Scheme 6. The starting N-Boc protected aminoacid (12) is converted into the corresponding Weinreb amide (13) using N,O-dimethylhydroxylamine hydrochloride in the presence of coupling reagents such as EDCI, HOBt, NMM in an appropriate solvent such as DCM or DMF. The Weinreb amide (13) may be transformed into a corresponding ketone (14) by action of a Grignard reagent in an appropriate solvent such as ether or THF under N₂ atmosphere. Then final deprotection of Boc group using either TFA or HCl may provide a corresponding aminoketone (10) or (15) in a salt form.

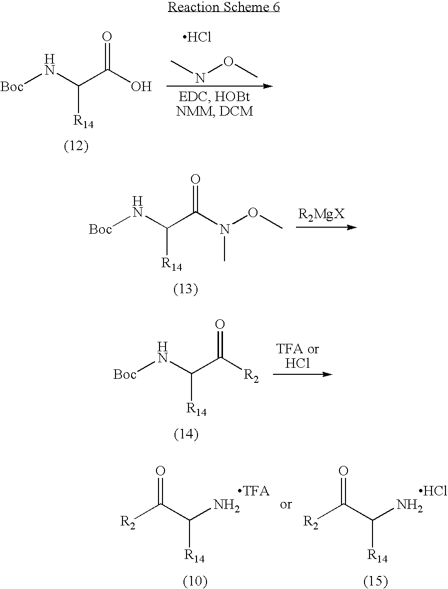

wherein R₂ and R₁₄ have the same meanings as defined above.

The compound of formula (Id) may be prepared by (i) reacting a carboxylic acid intermediate (5) with an aminoketone salt (10) or (15) in the presence of a coupling reagent, e. g. EDCI, DMAP, and (ii) cyclizing the resulting product (11) using a Lawesson's reagent, which can be conducted with microwave irradiation (See Kiryanov, A. A., Sampson, P., Seed, A. J., J. Org. Chem. 2001, 665, 7925-7929), as shown in Reaction Scheme 7.

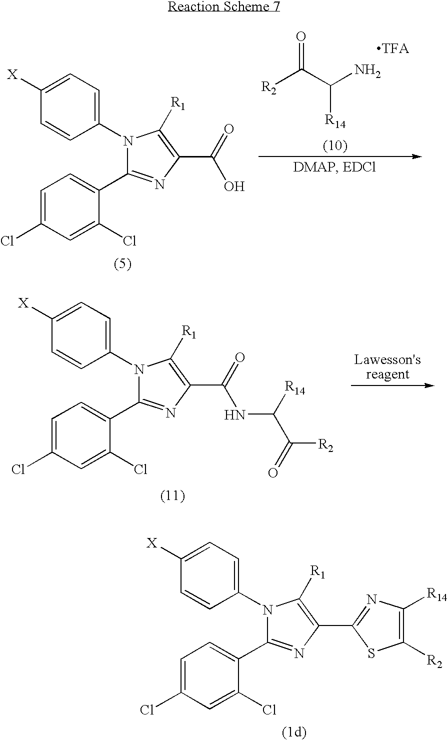

wherein R₁, R₂ and R₁₄ have the same meanings as defined above.

Further, the compound of formula (Ia) wherein R₁ is CH₃ may be treated with N-bromosuccinimide (NBS) in the presence of AIBN to produce a bromomethyl congener (16) (See Lange, J. H. M. et al, J. Med. Chem. 2005, 48, 1823-1838)., which is subjected to substitution of the bromo substituent by an azole such as 1,2,4-triazole in the presence of cesium carbonate led to a compound of formula (Ia-1), as shown in Reaction Scheme 8.

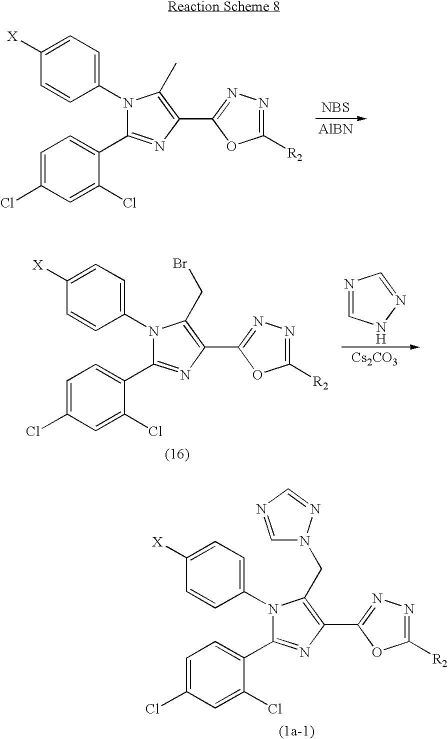

wherein R₂ has the same meaning as defined above.

Similarly, the compound of formula (Ib) wherein R₁ is CH₃ may be treated with N-bromosuccinimide (NBS) in the presence of AIBN to produce a bromomethyl congener (17) (See Lange, J. H. M. et al, J. Med. Chem. 2005, 48, 1823-1838)., which is subjected to substitution of the bromo substituent by an azole such as 1,2,4-triazole in the presence of cesium carbonate led to a compound of formula (Ib-1), as shown in Reaction Scheme 9.

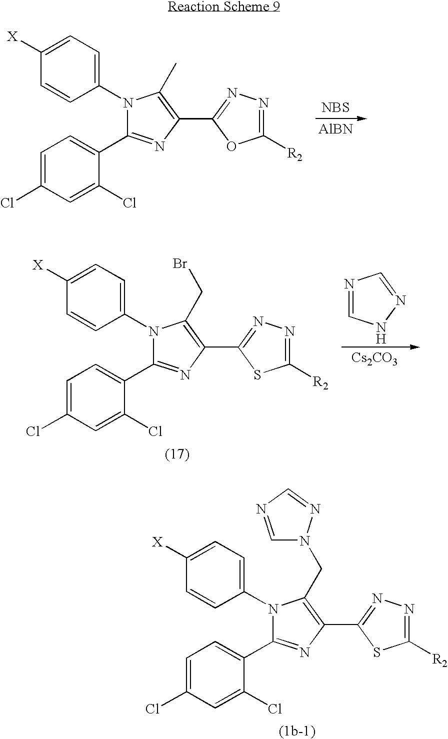

wherein R₂ has the same meaning as defined above.

The inventive heteroaryl-imidazole compound of formula (I) is effective as a cannabinoid CB₁ receptor inverse agonist or antagonist, thereby preventing or treating obesity and obesity-related metabolic disorders.

Accordingly, the present invention provides a pharmaceutical composition for preventing or treating obesity and obesity-related metabolic disorders, which comprises the compound of formula (I) or a pharmaceutically acceptable salt thereof as an active ingredient and a pharmaceutically acceptable carrier.

Further, the present invention provides a method for preventing or treating obesity and obesity-related metabolic disorders in a mammal, which comprises administering the compound of formula (I) to the mammal.

Also, the present invention provides a method for inhibiting cannabinoid CB₁ receptor in a mammal, which comprises administering the compound of formula (I) to the mammal.

As used herein, the term “obesity-related metabolic disorders” refers to chronic diseases that require treatment to reduce the excessive health risks associated with obesity and exemplary disorders include type 2 diabetes mellitus, cardiovascular and hypertension, hyperlipidaemia, fibrinolytic abnormalities.

The pharmaceutical composition may be administered orally, intramuscularly or subcutaneously. The formulation for oral administration may take various forms such as a syrup, tablet, capsule, cream and lozenge. A syrup formulation will generally contain a suspension or solution of the compound or its salt in a liquid carrier, e.g., ethanol, peanut oil, olive oil, glycerine or water, optionally with a flavoring or coloring agent. When the composition is in the form of a tablet, any one of pharmaceutical carriers routinely used for preparing solid formulations may be used. Examples of such carriers include magnesium stearate, terra alba, talc, gelatin, acacia, stearic acid, starch, lactose and sucrose. When the composition is in the form of a capsule, any of the routine encapsulation procedures may be employed, e.g., using the aforementioned carriers in a hard gelatin capsule shell. When the composition is formulated in the form of a soft gelatin shell capsule, any of the pharmaceutical carrier routinely used for preparing dispersions or suspensions may be prepared using an aqueous gum, cellulose, silicate or oil. The formulation for intramuscular or subcutaneous administration may take a liquid form such as a solution, suspension and emulsion which includes aqueous solvents such as water, physiological saline and Ringer's solution; or lipophilic solvents such as fatty oil, sesame oil, corn oil and synthetic fatty acid ester.

Preferably the composition is formulated in a specific dosage form for a particular patient.

Each dosage unit for oral administration contains suitably from 0.1 mg to 500 mg/Kg, and preferably from 1 mg to 100 mg/Kg of the compound of formula (I) or its pharmaceutically acceptable salt.

The suitable daily dosage for oral administration is about 0.01 mg/Kg to 40 mg/Kg of the compound of formula (I) or its pharmaceutically acceptable salt, may be administered 1 to 6 times a day, depending on the patient's condition.

The present invention is further described and illustrated in Examples provided below, which are, however, not intended to limit the scope of the present invention.

### EXAMPLE

As used herein the symbols and conventions used describing the processes, schemes and examples of the present invention are consistent with those used in the contemporary scientific literature, for example, the Journal of the American Chemical Society or the Journal of Biological Chemistry.

Unless otherwise noted, all starting materials were obtained from commercial suppliers and used without further purification.

Hz (Hertz)

Tᵣ (retention time)

MeOH (methanol)

TFA (trifluoroacetic acid)

EtOH (ethanol)

DMSO (dimethylsulfoxide)

DCM (dichloromethane)

DMF (N,N-dimethylformamide)

CDI (1,1-carbonyldiimidazole)

HOSu (N-hydroxysuccinimide)

HOBT (1-hydroxybenzotriazole)

Boc (tert-butyloxycarbonyl)

mCPBA (meta-chloroperbenzoic acid)

FMOC (9-fluorenylmethoxycarbonyl)

DCC (dicyclohexylcarbodiimide)

Cbz (benzyloxycarbonyl)

NMM (N-methyl morpholine)

HOAt (1-hydroxy-7-azabenzotriazole)

TBAF (tetra-n-butylammonium fluoride)

THP (tetrahydro-2H-pyran-2-yl)

DMAP (4-dimethylaminopyridine)

HPLC (high pressure liquid chromatography)

BOP (bis(2-oxo-3-oxazolidinyl)phosphinic chloride);

EDCI (1-ethyl-3-[3-dimethylaminopropyl]carbodiimide hydrochloride)

HBTU (O-Benzotriazolel-yl-N,N,N′,N′-tetramethyluronium hexafluorophosphate)

NBS (N-bromosuccinimide)

AIBN (2,2′-azobis(2-methylpropionitrile))

TLC (thin layer chromatography)TLC (thin layer chromatography)

RP (reverse phase)

i-PrOH (isopropanol)

TEA (triethylamine)

THF (tetrahydrofuran)

EtOAc (ethyl acetate)

HOAc (acetic acid)

Ac (acetyl)

Bn (benzyl)

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted, and all solvents are of the highest available purity unless otherwise indicated.

Microwave reaction was conducted with a Biotage microwave reactor.

¹H NMR spectra were recorded on either a Jeol ECX-400, or a Jeol JNM-LA300 spectrometer. Chemical shifts were expressed in parts per million (ppm, δ units). Coupling constants are in units of hertz (Hz). Splitting patterns describe apparent multiplicities and are designated as s (singlet), d(doublet), t (triplet), q (quartet), quint (quintet), m (multiplet), br (broad).

Mass spectra were obtained with either a Micromass, Quattro LC Triple Quadruple Tandem Mass Spectometer, ESI or Agilent, 1100LC/MSD, ESI.

For preparative HPLC, ca 100 mg of a product was injected in 1 mL of DMSO onto a SunFire™ Prep C18 OBD 5 um 19×100 mm Column with a 10 min gradient from 10% CH₃CN to 90% CH₃CN in H₂O. Flash chromatography was carried using Merck silica gel 60 (230-400 mesh). Most of the reactions were monitored by thin-layer chromatography on 0.25 mm E. Merck silica gel plates (60F-254), visualized with UV light using a 5% ethanolic phosphomolybdic acid or p-anisaldehyde solution.

The following synthetic schemes are merely illustrative of the methods by which the compounds of the invention may be prepared and are not intended to limit the scope of the invention as defined in the appended claims.

### Preparation of 1,3,4-oxadiazole (Formula (Ia))

### Example 1

### 2-tert-Butyl-5-(1-(4-chlorophenyl)-2-(2,4-dichlorophenyl)-5-methyl-1H-imidazol-4-yl)-1,3,4-oxadiazole

### Step1: 1-(4-chlorophenyl)-2-(2,4-dichlorophenyl)-5-methyl-N′-pivaloyl-1H-imidazole-4-carbohydrazide

To a solution of 1-(4-chlorophenyl)-2-(2,4-dichlorophenyl)-5-methyl-1H-imidazole-4-carboxylic acid (0.20 g, 0.524 mmol), pivalohydrazide (73 mg, 0.629 mmol), EDCI (0.24 g, 1.26 mmol) and HOBt (85 mg, 0.629 mmol) dissolved in DCM (5 ml) was added NMM (0.32 g, 3.15 mmol) in one portion at rt. The reaction mixture was stirred at rt for 18 hr. The organic layer was 5 collected, and evaporated under a vacuum. The crude mixture was further purified by preparative HPLC, to obtain 102 mg (0.213 mmol, 41%) of the title compound as a yellow solid.

¹H NMR (400 MHz, CDCl₃) ) δ 8.14 (br, s, 1H), 7.36-7.34 (m, 2H), 7.33 (d, J=1.8 Hz, 1H), 7.28 (d, J=8.2 Hz, 1H), 7.24 (dd, J=8.2, 1.8 Hz, 1H), 7.05-7.03 (m, 2H), 2.46 (s, 3H), 1.31 (s, 9H).

MH+479.

### Step2: 2-tert-butyl-5-(1-(4-chlorophenyl)-2-(2,4-dichlorophenyl)-5-methyl-1H-imidazol-4-yl)-1,3,4-oxadiazole

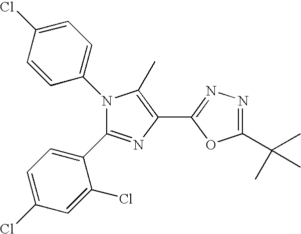

1-(4-chlorophenyl)-2-(2,4-dichlorophenyl)-5-methyl-N′-pivaloyl-1H-imidazole-4-carbohydrazide (40 mg, 0.083 mmol) obtained in Step 1 was added to a microwave reactor containing Burgess reagent (60 mg, 0.250 mmol) in THF (1 mL). The capped reactor was placed in a microwave reactor and the mixture was irradiated at 160° C. for 30 min. The reaction product was purified by preparative HPLC to provide the title compound (14.2 mg, 0.031 mmol, 37%) as a yellow solid.

¹H NMR (400 MHz, CDCl₃) δ 7.38-7.36 (m, 3H), 7.32 (d, J=2.3 Hz, 1H), 7.25 (dd, J=8.2, 2.3 Hz, 1H), 7.10-7.08 (m, 2H), 2.53 (s, 3H), 1.50 (s, 9H).

MH+461.

The following compounds of Examples 2 to 89 were obtained by repeating the procedure of Example 1.

### Example 2

### 2-(1-(4-Chlorophenyl)-2-(2,4-dichlorophenyl)-5-methyl-1H-imidazol-4-yl)-5-cyclohexyl-1,3,4-oxadiazole

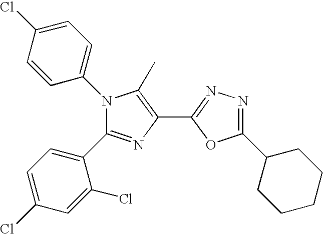

¹H NMR (400 MHz, CDCl₃) δ 7.39-7.34 (m, 3H), 7.32 (d, J=2.3 Hz, 1H), 7.26-7.23 (m, 1H), 7.09 (d, J=8.7 Hz, 2H), 3.03-2.95 (m, 1H), 2.54 (s, 3H), 2.16-2.12 (m, 2H), 1.88-1.84 (m, 2H), 1.77-1.67 (m, 3H), 1.44-1.25 (m, 3H).

MH+487.

### Example 3

### 2-(1-(4-Chlorophenyl)-2-(2,4-dichlorophenyl)-5-methyl-1H-imidazol-4-yl)-5-cyclopentyl-1,3,4-oxadiazole

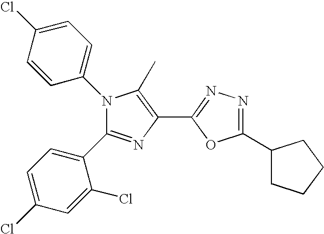

¹H NMR (400 MHz, CDCl₃) δ 7.39-7.35 (m, 3H), 7.32 (d, J=1.8 Hz, 1H), 7.25 (dd, J=8.2, 2.3 Hz, 1H), 7.10 (d, J=8.7 Hz, 2H), 3.43-3.35 (m, 1H), 2.54 (s, 3H), 2.18-2.10 (m, 2H), 2.08-1.99 (m, 2H), 1.89-1.79 (m, 2H), 1.75-1.66 (m, 2H).

MH+473.

### Example 4

### 2-(1-(4-Chlorophenyl)-2-(2,4-dichlorophenyl)-5-methyl-1H-imidazol-4-yl)-5-cyclobutyl-1,3,4-oxadiazole

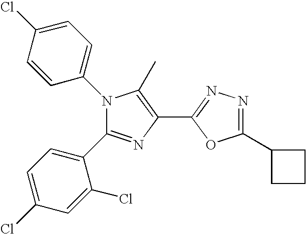

¹H NMR (400 MHz, CDCl₃) δ 7.39-7.34 (m, 3H), 7.33 (d, J=1.8 Hz, 1H), 7.26-7.23 (dd, J=8.2, 1.8 Hz, 1H), 7.10 (d, J=8.7 Hz, 2H), 3.85-3.77 (m, 1H), 2.59-2.52 (m, 5H), 2.48-2.40 (m, 2H), 2.18-1.99 (m, 2H).

MH+459.

### Example 5

### 2-(1-(4-Chlorophenyl)-2-(2,4-dichlorophenyl)-5-methyl-1H-imidazol-4-yl)-5-cycloheptyl-1,3,4-oxadiazole

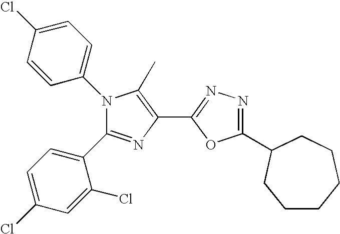

¹H NMR (400 MHz, CDCl₃) δ 7.39-7.34 (m, 3H), 7.32 (d, J=2.3 Hz, 1H), 7.24 (dd, J=8.2, 2.3 Hz, 1H), 7.11-7.07 (m, 2H), 3.22-3.15 (m, 1H), 2.54 (s, 3H), 2.20-2.14 (m, 2H), 1.99-1.89 (m, 2H), 1.85-1.79 (m, 2H), 1.68-1.53 (m, 6H).

MH+501.

### Example 6

### 2-(1-(4-Chlorophenyl)-2-(2,4-dichlorophenyl)-5-methyl-1H-imidazol-4-yl)-5-(hexan-2-yl)-1,3,4-oxadiazole

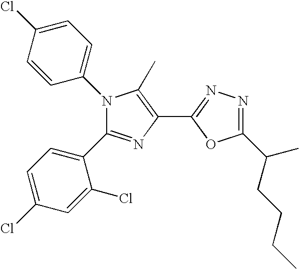

¹H NMR (400 MHz, CDCl₃) δ 7.39-7.35 (m, 3H), 7.33 (d, J=1.8 Hz, 1H), 7.25 (dd, J=8.2, 1.8 Hz, 1H), 7.11-7.07 (m, 2H), 3.21-3.12 (m, 1H), 2.54 (s, 3H), 1.97-1.88 (m, 1H), 1.73-1.64 (m, 1H), 1.42 (d, J=6.9 Hz, 3H), 1.37-1.28(m, 4H), 0.89 (t, J=7.3 Hz, 3H).

MH+489.

### Example 7

### 2-(1-(4-Chlorophenyl)-2-(2,4-dichlorophenyl)-5-methyl-1H-imidazol-4-yl)-5-(pentan-2-yl)-1,3,4-oxadiazole

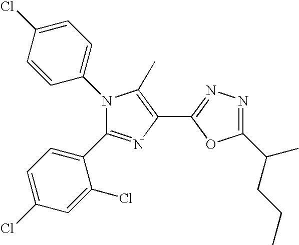

¹H NMR (400 MHz, CDCl₃) δ 7.39-7.35 (m, 3H), 7.32 (d, J=2.3 Hz, 1H), 7.25 (dd, J=8.2, 2.3 Hz, 1H), 7.11-7.07 (m, 2H), 3.23-3.14 (m, 1H), 2.54 (s, 3H), 1.96-1.87 (m, 1H), 1.71-1.61 (m, 1H), 1.44-1.32 (m, 5H), 0.92 (t, J=7.3 Hz, 3H).

MH+475.

### Example 8

### 2-sec-Butyl-5-(1-(4-chlorophenyl)-2-(2,4-dichlorophenyl)-5-methyl-1H-imidazol-4-yl)-1,3,4-oxadiazole

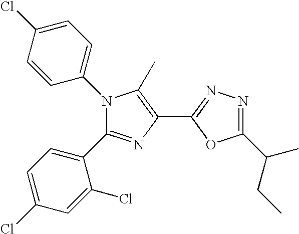

¹H NMR (400 MHz, CDCl₃) δ 7.39-7.35 (m, 3H), 7.33 (d, J=1.8 Hz, 1H), 7.25 (dd, J=8.2, 1.8 Hz, 1H), 7.11-7.07 (m, 2H), 3.15-3.06 (m, 1H), 2.54 (s, 3H), 2.01-1.90 (m, 1H), 1.80-1.69 (m, 1H), 1.43 (d, J=7.3 Hz, 3H), 0.97 (t, J=7.3 Hz, 3H).

MH+461.

### Example 9

### 2-(1-(4-Chlorophenyl)-2-(2,4-dichlorophenyl)-5-methyl-1H-imidazol-4-yl)-5-isopropyl-1,3,4-oxadiazole

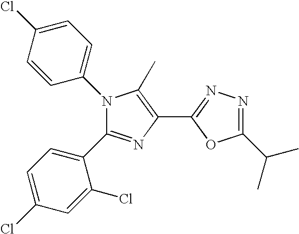

¹H NMR (400 MHz, CDCl₃) δ 7.39-7.35 (m, 3H), 7.33 (d, J=1.8 Hz, 1H), 7.25 (dd, J=8.2, 2.3 Hz, 1H), 7.11-7.08 (m, 2H), 3.33-3.25 (m, 1H), 2.54 (s, 3H), 1.46 (d, J=6.9 Hz, 6H).

MH+447.

### Example 10

### 2-(1-(4-Chlorophenyl)-2-(2,4-dichlorophenyl)-5-methyl-1H-imidazol-4-yl)-5-(pentan-3-yl)-1,3,4-oxadiazole

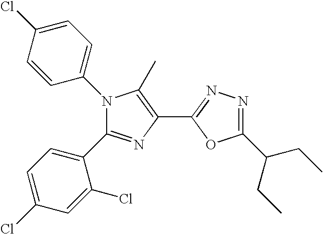

¹H NMR (400 MHz, CDCl₃) δ 7.39-7.32 (m, 4H), 7.27-7.24 (m, 1H), 7.10-7.08 (m, 2H), 2.97-2.93 (m, 1H), 2.55 (s, 3H), 1.93-1.76 (m, 4H), 0.93 (t, J=7.2 Hz, 6H).

MH+475.

### Example 11

### 2-(1-(4-Chlorophenyl)-2-(2,4-dichlorophenyl)-5-methyl-1H-imidazol-4-yl)-5-(1-phenylethyl)-1,3,4-oxadiazole

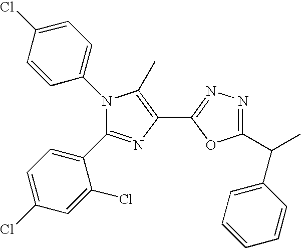

¹H NMR (400 MHz, CDCl₃) δ 7.38-7.33 (m, 5H), 7.32-7.30 (m, 3H), 7.27-7.22 (m, 2H), 7.09-7.05 (m, 2H), 4.44 (q, J=7.3 Hz, 1H), 2.51 (s, 3H), 1.82 (d, J=7.3 Hz, 3H).

MH+509.

### Example 12

### 2-(1-(4-Chlorophenyl)-2-(2,4-dichlorophenyl)-5-ethyl-1H-imidazol-4-yl)-5-cyclohexyl-1,3,4-oxadiazole

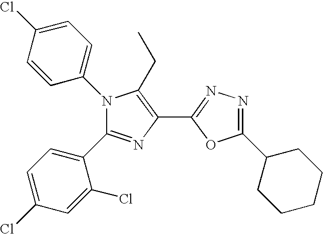

¹H NMR (400 MHz, CDCl₃) δ 7.39-7.32 (m, 4H), 7.23 (dd, J=8.2, 1.8 Hz, 1H), 7.15-7.12 (m, 2H), 3.03-2.97 (m, 3H), 2.15 (d, J=11 Hz, 2H), 1.87-1.84 (m, 2 H), 1.76-1.67 (m, 3H), 1.44-1.25 (m, 3H), 1.10 (t, J=7.8 Hz, 3H).

MH+501.

### Example 13

### 2-(1-(4-Chlorophenyl)-2-(2,4-dichlorophenyl)-5-ethyl-1H-imidazol-4-yl)-5-cyclopentyl-1,3,4-oxadiazole

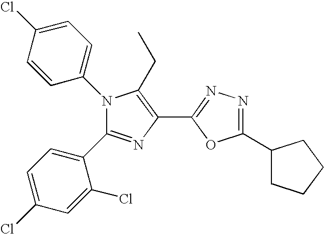

¹H NMR (400 MHz, CDCl₃) δ 7.39-7.32 (m, 4H), 7.23 (dd, J=8.2, 1.8 Hz, 1H), 7.13 (d, J=8.2 Hz, 2H), 3.43-3.34 (m, 1H), 3.00 (q, J=7.3 Hz, 2H), 2.19-2.00 (m, 4H), 1.89-1.79 (m, 2 H), 1.75-1.67 (m, 2H), 1.10 (t, J=7.3 Hz, 3H).

MH+487.

### Example 14

### 2-(1-(4-Chlorophenyl)-2-(2,4-dichlorophenyl)-5-ethyl-1H-imidazol-4-yl)-5-cyclobutyl-1,3,4-oxadiazole

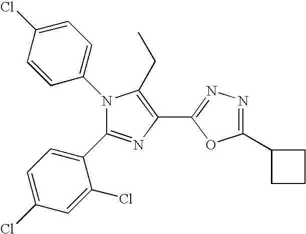

¹H NMR (400 MHz, CDCl₃) δ 7.40-7.32 (m, 4H), 7.23 (dd, J=8.2, 1.8 Hz, 1H), 7.16-7.12 (m, 2H), 3.86-3.77 (m, 1H), 3.01 (q, J=7.3 Hz, 2H), 2.62-2.52 (m, 2H), 2.47-2.39 (m, 2 H), 2.16-2.01 (m, 2H), 1.11 (t, J=7.3 Hz, 3H).

MH+473.

### Example 15

### 2-tert-butyl-5-(1-(4-chlorophenyl)-2-(2,4-dichlorophenyl)-5-ethyl-1H-imidazol-4-yl)-1,3,4-oxadiazole

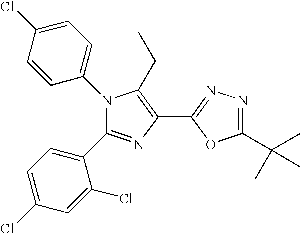

¹H NMR (300 MHz, CDCl₃) δ 7.36-7.30 (m, 4H), 7.27-7.24 (m, 1H), 7.14-7.11 (m, 2H), 2.98 (q, J=7.3 Hz, 2H), 1.50 (s, 9H), 1.11 (t, J=7.3 Hz, 3H).

MH+475.

### Example 16

### 2-(1-(4-Chlorophenyl)-2-(2,4-dichlorophenyl)-5-ethyl-1H-imidazol-4-yl)-5-(hexan-2-yl)-1,3,4-oxadiazole

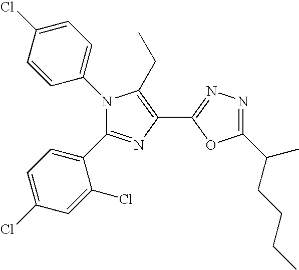

¹H NMR (300 MHz, CDCl₃) δ 7.40-7.32 (m, 4H), 7.24 (dd, J=8.2, 2.2 Hz, 1H), 7.15-7.12 (m, 2H), 3.20-3.11 (m, 1H), 3.00 (q, J=7.3 Hz, 2H), 1.97-1.90 (m, 1H), 1.71-1.61 (m, 1H), 1.42 (d, J=7.0 Hz, 3H), 1.35-1.32 (m, 4H), 1.11 (t, J=7.3 Hz, 3H), 0.89 (t, J=6.8 Hz, 3H).

MH+503.

### Example 17

### 2-(1-(4-Chlorophenyl)-2-(2,4-dichlorophenyl)-5-ethyl-1H-imidazol-4-yl)-5-(pentan-2-yl)-1,3,4-oxadiazole

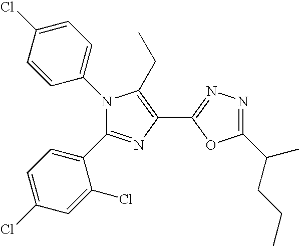

¹H NMR (400 MHz, CDCl₃) δ 7.39-7.32 (m, 4H), 7.24 (dd, J=8.4, 2.0 Hz, 1H), 7.14-7.11 (m, 2H), 3.21-3.16 (m, 1H), 3.00 (q, J=7.6 Hz, 2H), 1.93-1.89 (m, 1H), 1.69-1.60 (m, 1H), 1.41-1.35 (m, 5H), 1.11 (t, J=7.6 Hz, 3H), 0.92 (t, J=7.2 Hz,3H).

MH+489.

### Example 18

### 2-sec-Butyl-5-(1-(4-chlorophenyl)-2-(2,4-dichlorophenyl)-5-ethyl-1H-imidazol-4-yl)-1,3,4-oxadiazole

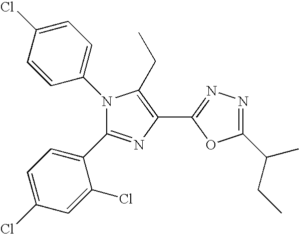

¹H NMR (400 MHz, CDCl₃) δ 7.40-7.34 (m, 3H), 7.32 (d, J=2.0 Hz, 1H), 7.24 (dd, J=8.4, 2.0 Hz, 1H), 7.15-7.12 (m, 2H), 3.13-3.08 (m, 1H), 3.01 (q, J=7.6 Hz, 2H), 1.99-1.92 (m, 1H), 1.78-1.71 (m, 1H), 1.43 (d, J=7.2 Hz, 3H), 1.11 (t, J=7.2 Hz, 3H), 0.97 (t, J=7.6 Hz, 3H).

MH+475.

### Example 19

### 2-(1-(4-Bromophenyl)-2-(2,4-dichlorophenyl)-5-methyl-1H-imidazol-4-yl)-5-cyclohexyl-1,3,4-oxadiazole

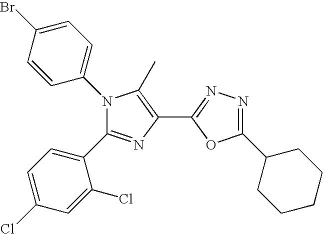

¹H NMR (400 MHz, CDCl₃) δ 7.53 (d, J=8.8 Hz, 2H), 7.36 (d, J=8.0 Hz, 1H), 7.33 (d, J=1.6 Hz, 1H), 7.27-7.24 (m, 1H), 7.03 (d, J=8.4 Hz, 2H), 3.03-2.95 (m, 1H), 2.54 (s, 3H), 2.16-2.13 (m, 2H), 1.88-1.84 (m, 2H), 1.77-1.67 (m, 3H), 1.45-1.26 (m, 3H).

MH+531.

### Example 20

### 2-(1-(4-Bromophenyl)-2-(2,4-dichlorophenyl)-5-methyl-1H-imidazol-4-yl)-5-tert-butyl-1,3,4-oxadiazole

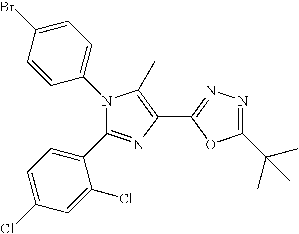

¹H NMR (400 MHz, CDCl₃) δ 7.54-7.51 (m, 2H), 7.38-7.24 (m, 3H), 7.04-7.01 (m, 2H), 2.53 (s, 3H), 1.50 (s, 9H).

MH+505.

### Example 21

### 2-(1-(4-Bromophenyl)-2-(2,4-dichlorophenyl)-5-ethyl-1H-imidazol-4-yl)-5-tert-butyl-1,3,4-oxadiazole

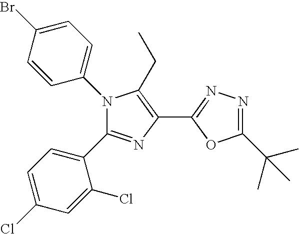

¹H NMR (400 MHz, CDCl₃) δ 7.54-7.52 (m, 2H), 7.37-7.35 (m, 1H), 7.32 (m, 1H), 7.24 (dd, J=8.4, 2.0 Hz, 1H), 7.08-7.05 (m, 2H), 2.99 (q, J=7.2 Hz, 2H), 1.50 (s, 9H), 1.11 (t, J=7.6 Hz, 31H).

MH+519.

### Example 22

### 2-(1-(4-Bromophenyl)-2-(2,4-dichlorophenyl)-5-methyl-1H-imidazol-4-yl)-5-cyclobutyl-1,3,4-oxadiazole

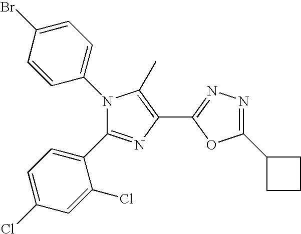

¹H NMR (400 MHz, CDCl₃) δ 7.54 (d, J=8.8 Hz, 2H), 7.36 (d, J=8.4 Hz, 1H), 7.33 (d, J=2.0 Hz, 1H), 7.26-7.24 (m, 1H), 7.03 (d, J=8.8 Hz, 2H), 3.86-3,77 (m, 1H), 2.62-2.52 (m, 5H), 2.47-2.39 (m, 2H), 2.16-2.00 (m, 2H).

MH+503.

### Example 23

### 2-(1-(4-Bromophenyl)-2-(2,4-dichlorophenyl)-5-ethyl-1H-imidazol-4-yl)-5-cyclobutyl-1,3,4-oxadiazole

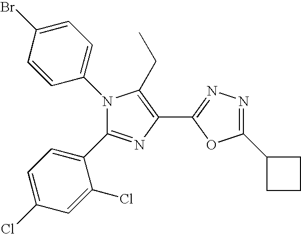

¹H NMR (400 MHz, CDCl₃) δ 7.54 (d, J=8.4 Hz, 2H), 7.36-7.33 (m, 2H), 7.24 (dd, J=8.0, 2.0 Hz, 1H), 7.07 (d, J=8.4 Hz, 2H), 3.86-3.79 (m, 1H), 3.01 (q, J=7.6 Hz, 2H), 2.60-2.52 (m, 2H), 2.47-2.40 (m, 2H), 2.16-2.01 (m, 2H), 1.11 (t, J=7.6 Hz,3H).

MH+517.

### Example 24

2-tert-Butyl-5-(1-(4-chlorophenyl)-5-cyclopropyl-2-(2,4-dichlorophenyl)-1H-imidazol-4-yl)-1,3,4-oxadiazole

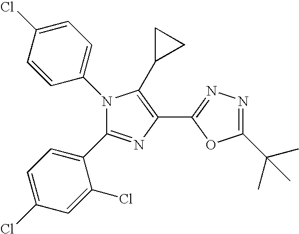

¹H NMR (400 MHz, CDCl₃) δ 7.40-7.34 (m, 3H), 7.31 (d, J=2.4 Hz, 1H), 7.27-7.24 (m, 1H), 7.11 (d, J=8.8 Hz, 2H), 1.90-1.85 (m, 1H), 1.50 (s, 9H), 0.92-0.87 (m, 2H), 0.59-0.55 (m, 2H).

MH+487.

### Example 25

### 2-(1-(4-Bromophenyl)-2-(2,4-dichlorophenyl)-5-isopropyl-1H-imidazol-4-yl)-5-tert-butyl-1,3,4-oxadiazole

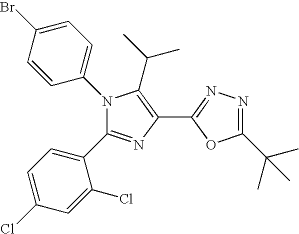

¹H NMR (400 MHz, CDCl₃) δ 7.52 (d, J=8.8 Hz, 2H), 7.32-7.30 (m, 2H), 7.22 (dd, J=8.4, 2.0 Hz, 1H), 7.07 (d, J=8.8 Hz, 2H), 3.39-3.32 (m, 1H), 1.50 (s, 9H), 1.34 (d, J=7.2 Hz, 6H).

MH+533.

### Example 26

### 2-(1-(4-Bromophenyl)-2-(2,4-dichlorophenyl)-5-isopropyl-1H-imidazol-4-yl)-5-(1-phenylcyclopropyl)-1,3,4-oxadiazole

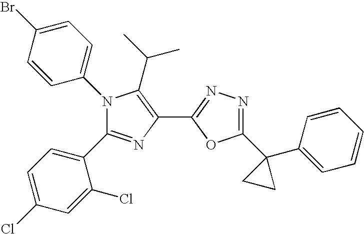

¹H NMR (400 MHz, CDCl₃) δ 7.52-7.47 (m, 4H), 7.36-7.27 (m, 5H), 7.20 (dd, J=8.4, 2.0 Hz, 1H), 7.04 (d, J=8.4 Hz, 2H), 3.27-3.20 (m, 1H), 1.78 (dd, J=6.8, 4.8 Hz, 2H), 1.46 (dd, J=7.2, 4.4 Hz, 2H), 1.26 (d, J=7.6 Hz, 6H).

MH+593.

### Example 27

### 2-(1-(4-Bromophenyl)-2-(2,4-dichlorophenyl)-5-ethyl-1H-imidazol-4-yl)-5-(1-phenylcyclopropyl)-1,3,4-oxadiazole

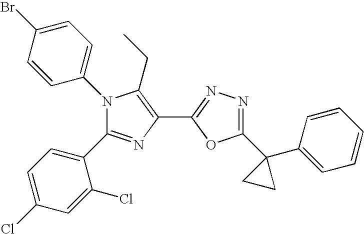

¹H NMR (400 MHz, CDCl₃) δ 7.52 (d, J=8.8 Hz, 2H), 7.49-7.46 (m, 2H), 7.36-7.28 (m, 5H), 7.22 (dd, J=8.0, 2.0 Hz, 1H), 7.03 (d, J=8.8 Hz, 2H), 2.90 (q, J=7.6 Hz, 2H), 1.77 (dd, J=6.8, 4.8 Hz, 2H), 1.46 (dd, J=7.2, 4.4 Hz, 2H), 1.03 (t, J=7.6 Hz, 3H).

MH+579.

### Example 28

### 2-(1-(4-Chlorophenyl)-2-(2,4-dichlorophenyl)-5-isopropyl-1H-imidazol-4-yl)-5-(1-phenylcyclopropyl)-1,3,4-oxadiazole

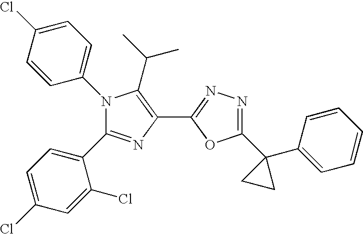

¹H NMR (400 MHz, CDCl₃) δ 7.47 (d, J=6.8 Hz, 2H), 7.35-7.25 (m, 7H), 7.19 (d, J=7.6 Hz, 1H), 7.10 (d, J=8.4 Hz, 2H), 3.25-3.22 (m, 1H), 1.77 (dd, J=7.2, 4.8 Hz, 2H), 1.45 (dd, J=7.2, 4.8 Hz, 2H), 1.25 (d, J=6.8 Hz, 6H).

MH+549.

### Example 29

### 2-(1-(4-Bromophenyl)-2-(2,4-dichlorophenyl)-5-propyl-1H-imidazol-4-yl)-5-tert-butyl-1,3,4-oxadiazole

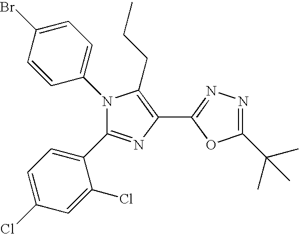

¹H NMR (400 MHz, CDCl₃) δ 7.52 (d, J=8.0 Hz, 2H), 7.36-7.29 (m, 2H), 7.25-7.22 (m, 1H), 7.04 (d, J=8.8 Hz, 2H), 2.93 (t, J=8.0 Hz, 2H), 1.54-1.49 (m, 11H), 0.85 (t, J=6.8 Hz, 3H).

MH+533.

### Example 30

### 2-(1-(4-Bromophenyl)-2-(2,4-dichlorophenyl)-5-propyl-1H-imidazol-4-yl)-5-cyclohexyl-1,3,4-oxadiazole

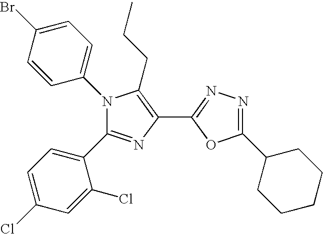

¹H NMR (400 MHz, CDCl₃) δ 7.54-7.50 (m, 2H), 7.36-7.30 (m, 2H), 7.26-7.21 (m, 1H), 7.05-7.01 (m, 2H), 3.79-3.67 (m, 2H), 3.58 (t, J=5.6 Hz, 1H), 2.98-2.88 (m, 2H), 2.14 (d, J=13.2 Hz, 1H), 1.86-1.61 (m, 4H), 1.53-1.21 (m, 5H), 0.86-0.81 (m, 3H).

MH+559.

### Example 31

### 2-(1-(4-Bromophenyl)-2-(2,4-dichlorophenyl)-5-propyl-1H-imidazol-4-yl)-5-(1-phenylcyclopropyl)-1,3,4-oxadiazole

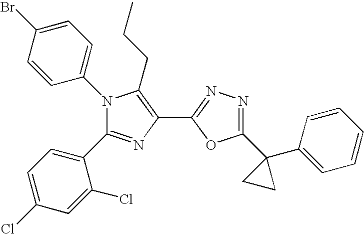

¹H NMR (400 MHz, CDCl₃) δ 7.53-7.49 (m, 3H), 7.48-7.37 (m, 3H), 7.35-7.33 (m, 2H), 7.25-7.23 (m, 1H), 7.19 (dd, J=8.4, 2.0 Hz, 1H), 6.98 (d, J=8.8 Hz, 2H), 2.81 (t, J=8.0 Hz, 2H), 1.69 (dd, J=6.8, 3.6 Hz, 2H), 1.45-1.36(m, 2H), 1.40 (dd, J=6.8, 3.6 Hz, 2H), 0.77 (t, J=7.2 Hz, 3H).

MH+593.

### Example 32

### 2-(1-(4-Bromophenyl)-2-(2,4-dichlorophenyl)-5-propyl-1H-imidazol-4-yl)-5-cyclobutyl-1,3,4-oxadiazole

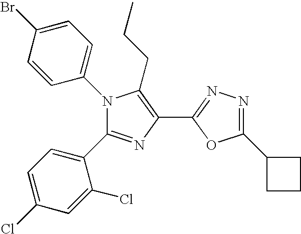

¹H NMR (400 MHz, CDCl₃) δ 7.52 (d, J=8.8 Hz, 2H), 7.34 (s, 1H), 7.32 (d, J=1.6 Hz, 1H), 7.22 (dd, J=8.4, 2.0 Hz, 1H), 7.05 (d, J=8.8 Hz, 2H), 3.82-3.77 (m, 1H), 2.95 (t, J =8.0 Hz, 2H), 2.58-2.51 (m, 2H), 2.47-2.40 (m, 2H), 2.14-2.01 (m, 2H), 1.53-1.47 (m, 2H), 0.84 (t, J=7.2 Hz, 3H).

MH+531.

### Example 33

### 2-(1-(4-Bromophenyl)-2-(2,4-dichlorophenyl)-5-propyl-1H-imidazol-4-yl)-5-(1-(4-chlorophenyl)cyclopropyl)-1,3,4-oxadiazole

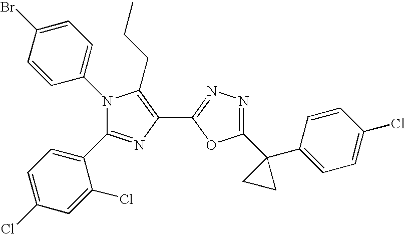

¹H NMR (400 MHz, CDCl₃) δ 7.50 (d, J=8.4 Hz, 2H), 7.41 (d, J=8.4 Hz, 2H), 7.32-7.29 (m, 4H), 7.19 (dd, J=8.4, 2.0 Hz, 1H), 7.01 (d, J=8.4 Hz, 2H), 2.85 (t, J=8.0 Hz, 2H), 1.79 (dd, J=6.8, 2.4 Hz, 2H), 1.44-1.41 (m, 4H), 0.79 (t, J=7.2 Hz, 3H).

MH+627.

### Example 34

### 2-(1-(4-Bromophenyl)-2-(2,4-dichlorophenyl)-5-propyl-1H-imidazol-4-yl)-5-(1-(4-chlorophenyl)cyclobutyl)-1,3,4-oxadiazole

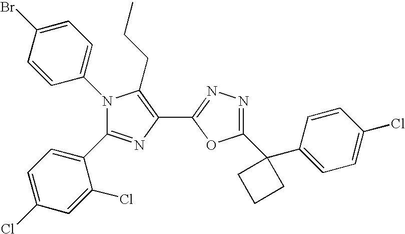

1H NMR (400 MHz, CDCl₃) δ 7.50 (d, J=8.0 Hz, 2H), 7.33-7.26 (m, 6H), 7.22 (dd, J=8.0, 2.0 Hz, 1H), 7.00 (d, J=8.4 Hz, 2H), 3.08-3.06 (m, 2H), 2.87 (t, J=8.0 Hz, 2H), 2.23-2.19 (m, 1H), 2.01-1.99 (m, 1H), 1.49-1.44 (m, 2H), 0.81 (t, J=7.2 Hz, 3H).

MH+641.

### Example 35

### 2-tert-Butyl-5-(1-(4-chlorophenyl)-2-(2,4-dichlorophenyl)-5-propyl-1H-imidazol-4-yl)-1,3,4-oxadiazole

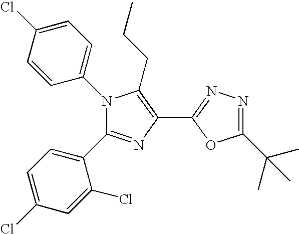

¹H NMR (400 MHz, CDCl₃) δ 7.37-7.33 (m, 3H), 7.31 (d, J=2.0 Hz, 1H), 7.22 (dd, J=8.4, 2.0 Hz, 1H), 7.10 (d, J=8.4 Hz, 2H), 2.92 (t, J=8.0 Hz, 2H), 1.53-1.48 (m, 11H), 0.84 (t, J=7.6 Hz, 3H).

MH+489.

### Example 36

### 2-(1-(4-Chlorophenyl)-2-(2,4-dichlorophenyl)-5-propyl-1H-imidazol-4-yl)-5-cyclobutyl-1,3,4-oxadiazole

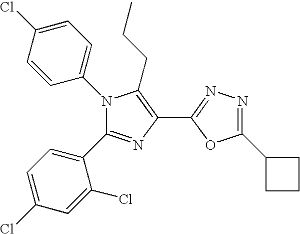

¹H NMR (400 MHz, CDCl₃) δ 7.37-7.31 (m, 4H), 7.22 (dd, J=8.4, 2.0 Hz, 1H), 7.11 (d, J=8.4 Hz, 2H), 3.82-3.77 (m, 1H), 2.95 (t, J=8.0 Hz, 2H), 2.58-2.51 (m, 2H), 2.45-2.40 (m, 2H), 2.14-2.00 (m, 2H), 1.53-1.47 (m, 2H), 0.84 (t, J=7.2 Hz, 3H).

MH+487.

### Example 37

### 2-(1-(4-Chlorophenyl)-2-(2,4-dichlorophenyl)-5-propyl-1H-imidazol-4-yl)-5-(1-(4-chlorophenyl)cyclopropyl)-1,3,4-oxadiazole

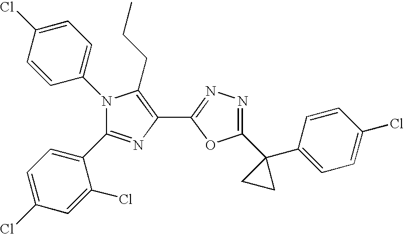

¹H NMR (400 MHz, CDCl₃) δ 7.41 (d, J=8.8 Hz, 2H), 7.36-7.29 (m, 6H), 7.22 (dd, J=8.4, 2.0 Hz, 1H), 7.08 (d, J=8.8 Hz, 2H), 2.85 (t, J=8.0 Hz, 2H), 1.79 (dd, J=7.2, 4.4 Hz, 2H), 1.47-1.39 (m, 4H), 0.78 (t, J=7.2 Hz, 3H).

MH+583.

### Example 38

### 2-(1-(4-Chlorophenyl)-2-(2,4-dichlorophenyl)-5-propyl-1H-imidazol-4-yl)-5-(1-(4-chlorophenyl)cyclobutyl)-1,3,4-oxadiazole

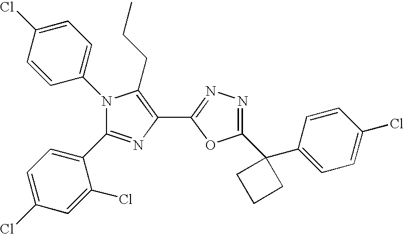

¹H NMR (400 MHz, CDCl₃) δ 7.42 (d, J=8.8 Hz, 2H), 7.35 (s, 1H), 7.32 (d, J=3.2 Hz, 2H), 7.30 (s, 1H), 7.28 (d, J=2.0 Hz, 1H), 7.23 (s, 1H), 7.18 (dd, J=8.4, 2.0 Hz, 1H), 7.08 (d, J=8.8 Hz, 2H), 2.95 (t, J=8.0 Hz, 2H), 1.89 (dd, J=7.2, 4.0 Hz, 2H), 1.51-1.46 (m, 6H), 0.82 (t, J=7.2 Hz, 3H).

MH+597.

### Example 39

### 2-(1-(4-Chlorophenyl)-2-(2,4-dichlorophenyl)-5-propyl-1H-imidazol-4-yl)-5-cyclohexyl-1,3,4-oxadiazole

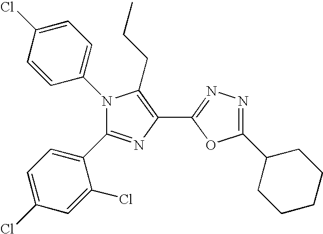

¹H NMR (400 MHz, CDCl₃) δ 7.38-7.34 (m, 2H), 7.32-7.31 (m, 2H), 7.22 (dd, J=8.4, 2.4 Hz, 1H), 7.11 (d, J=8.4 Hz, 2H), 3.00-2.92 (m, 3H), 2.16-2.12 (m, 2H), 1.86-1.82 (m, 2H), 1.75-1.68 (m, 2H), 1.53-1.47 (m, 2H), 1.41-1.24 (m, 4H), 0.84 (t, J=7.2 Hz, 3H).

MH+515.

### Example 40

### 2-(1-(4-Bromophenyl)-2-(2,4-dichlorophenyl)-5-ethyl-1H-imidazol-4-yl)-5-(1-(4-chlorophenyl)cyclopropyl)-1,3,4-oxadiazole

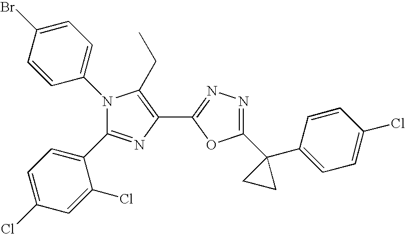

¹H NMR (400 MHz, CDCl₃) δ 7.52 (d, J=8.4 Hz, 2H), 7.42 (d, J=8.0 Hz, 2H), 7.32-7.29 (m, 4H), 7.23 (dd, J=8.4, 2.0 Hz, 1H), 7.04 (d, J=8.8 Hz, 2H), 2.91 (q, J=7.2 Hz, 2H), 1.79 (dd, J=7.2, 4.8 Hz, 2H), 1.43 (dd, J=7.2, 4.8 Hz, 2H), 1.05 (t, J=7.2 Hz, 3H).

MH+613.

### Example 41

### 2-(1-(4-Bromophenyl)-2-(2,4-dichlorophenyl)-5-ethyl-1H-imidazol-4-yl)-5-(1-(4-chlorophenyl)cyclobutyl)-1,3,4-oxadiazole

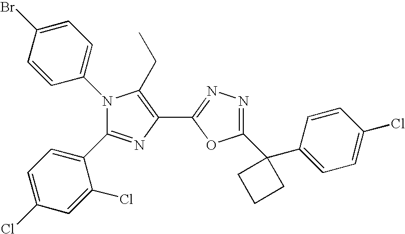

¹H NMR (400 MHz, CDCl₃) δ 7.51 (d, J=8.4 Hz, 2H), 7.34-7.28 (m, 6H), 7.22 (dd, J=8.4, 2.0 Hz, 1H), 7.03 (d, J=8.4 Hz, 2H), 3.11-3.06 (m, 2H), 2.93 (q, J=7.6 Hz, 2H), 2.75-2.72 (m, 2H), 2.25-2.22 (m, 1H), 2.06-1.99 (m, 1H), 1.06 (t, J=7.2 Hz, 3H).

MH+627.

### Example 42

### 2-(1-(4-Bromophenyl)-2-(2,4-dichlorophenyl)-5-ethyl-1H-imidazol-4-yl)-5-(1-(4-methoxyphenyl)cyclopropyl)-1,3,4-oxadiazole

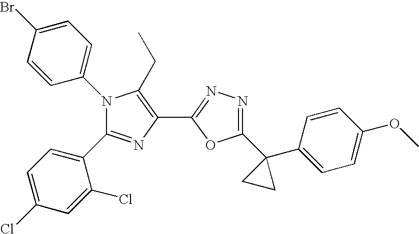

¹H NMR (400 MHz, CDCl₃) δ 7.51 (d, J=8.8 Hz, 2H), 7.41 (d, J=8.4 Hz, 2H), 7.32-7.30 (m, 2H), 7.22 (dd, J=8.0, 2.0 Hz, 1H), 7.02 (d, J=8.4 Hz, 2H), 6.87 (d, J=8.8 Hz, 2H), 3.79 (s, 3H), 2.91 (q, J=7.2 Hz, 2H), 1.75-1.73 (m, 2H), 1.42-1.40 (m, 2H), 1.03 (t, J=7.2 Hz, 3H).

MH+609.

### Example 43

### 2-(1-(4-Bromophenyl)-2-(2,4-dichlorophenyl)-5-ethyl-1H-imidazol-4-yl)-5-(1-p-tolylcyclopropyl)-1,3,4-oxadiazole

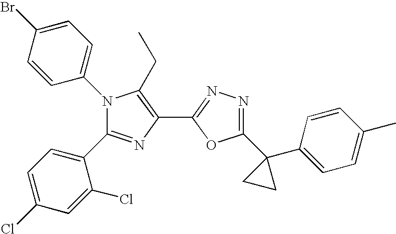

¹H NMR (400 MHz, CDCl₃) δ 7.51 (d, J=8.8 Hz, 2H), 7.36 (d, J=8.0 Hz, 2H), 7.33-7.30 (m, 2H), 7.20 (dd, J=7.6, 2.0 Hz, 1H), 7.14 (dd, J=8.4, 3.2 Hz, 2H), 7.02 (d, J=8.8 Hz, 2H), 2.89 (q, J=7.6 Hz, 2H), 2.32 (s, 3H), 1.74 (dd, J=7.2, 4.0 Hz, 2H), 1.45-1.40 (m, 2H), 1.03 (t, J=7.2 Hz, 3H).

MH+593.

### Example 44

### 2-(1-(4-Bromophenyl)-2-(2,4-dichlorophenyl)-5-ethyl-1H-imidazol-4-yl)-5-(1-(2,4-dichlorophenyl)cyclopropyl)-1,3,4-oxadiazole

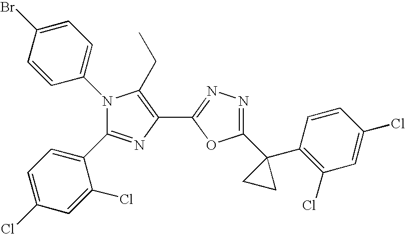

¹H NMR (400 MHz, CDCl₃) δ 7.51 (d, J=8.4 Hz, 2H), 7.45-7.42 (m, 2H), 7.33-7.30 (m, 2H), 7.27-7.24 (m, 2H), 7.02 (d, J=8.4 Hz, 2H), 2.88 (q, J=7.2 Hz, 2H), 1.96 (dd, J=7.2, 4.8 Hz, 2H), 1.47 (dd, J=7.2, 4.8 Hz, 2H), 1.02 (t, J=7.2 Hz, 3H).

MH+647.

### Example 45

### 2-(1-(4-Bromophenyl)-2-(2,4-dichlorophenyl)-5-ethyl-1H-imidazol-4-yl)-5-(1-(4-chlorophenyl)cyclopentyl)-1,3,4-oxadiazole

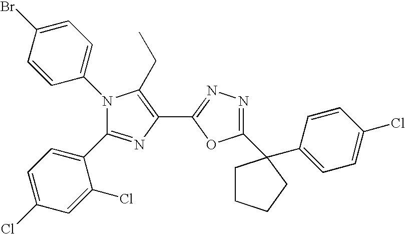

¹H NMR (400 MHz, CDCl₃) δ 7.50 (d, J=8.8 Hz, 2H), 7.36 (d, J=8.8 Hz, 2H), 7.32-7.24 (m, 4H), 7.21 (dd, J=8.0,2.0 Hz, 1H), 7.01 (d, J=8.8 Hz, 2H), 2.92 (q, J=7.6 Hz, 4H), 2.19-2.16 (m, 2H), 1.84-1.82 (m, 4H), 1.06 (t, J=7.2 Hz, 3H).

MH+641.

### Example 46

### 2-(1-(4-Bromophenyl)-2-(2,4-dichlorophenyl)-5-methyl-1H-imidazol-4-yl)-5-(1-(trifluoromethyl)cyclopropyl)-1,3,4-oxadiazole

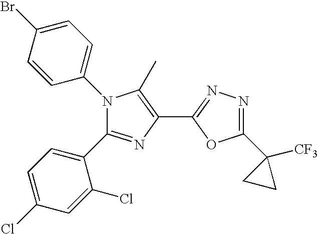

¹H NMR (400 MHz, CDCl₃) δ 7.54 (d, J=8.0 Hz, 2H), 7.38 (d, J=8.0 Hz, 1H), 7.32 (s, 1H), 7.28-7.25 (m, 1H), 7.02 (d, J=8.0 Hz, 2H), 2.53 (s, 3H), 1.67 (s, 2H), 1.60 (t, J=8.4 Hz, 2H).

MH+557.

### Example 47

### 2-(1-(4-Bromophenyl)-2-(2,4-dichlorophenyl)-5-methyl-1H-imidazol-4-yl)-5-(1-(trifluoromethyl)cyclobutyl)-1,3,4-oxadiazole

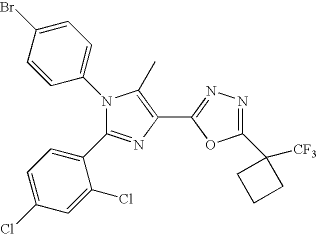

¹H NMR (400 MHz, CDCl₃) δ 7.54 (d, J=8.8 Hz, 2H), 7.37 (d, J=8.4 Hz, 1H), 7.32 (d, J=2.0 Hz, 1H), 7.27-7.24 (m, 1H), 7.03 (d, J=8.8 Hz, 2H), 2.88-2.74 (m, 4H), 2.54 (s, 3H), 2.20-2.11 (m, 2H).

MH+571.

### Example 48

### 2-(1-(4-Bromophenyl)-2-(2,4-dichlorophenyl)-5-methyl-1H-imidazol-4-yl)-5-(1-(4-chlorophenyl)cyclopropyl)-1,3,4-oxadiazole

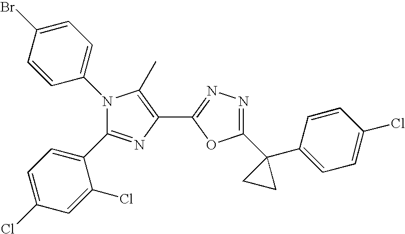

¹H NMR (400 MHz, CDCl₃) δ 7.52 (d, J=8.4 Hz, 2H), 7.41 (d, J=8.4 Hz, 2H), 7.35-7.30 (m, 4H), 7.26-7.23 (m, 1H), 7.00 (d, J=8.4 Hz, 2H), 2.48 (s, 3H), 1.78 (dd, J=6.8, 4.8 Hz, 2H), 1.43 (dd, J=6.8, 4.8 Hz, 2H).

MH+599.

### Example 49

### 2-(1-(4-Bromophenyl)-2-(2,4-dichlorophenyl)-5-ethyl-1H-imidazol-4-yl)-5-(1-(trifluoromethyl)cyclopropyl)-1,3,4-oxadiazole

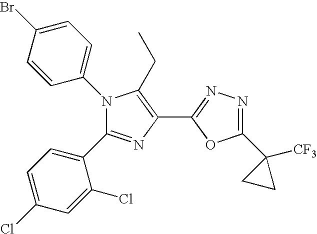

¹H NMR (400 MHz, CDCl₃) δ 7.54 (d, J=8.8 Hz, 2H), 7.36-7.32 (m, 2H), 7.26-7.23 (m, 1H), 7.06 (d, J=8.4 Hz, 2H), 2.97 (q, J=7.6 Hz, 2H), 1.63-1.61 (m, 2H), 1.61-1.60 (m, 2H), 1.11 (t, J=7.6 Hz, 3H).

MH+571.

### Example 50

### 2-(1-(4-Bromophenyl)-2-(2,4-dichlorophenyl)-5-ethyl-1H-imidazol-4-yl)-5-(1-(trifluoromethyl)cyclobutyl)-1,3,4-oxadiazole

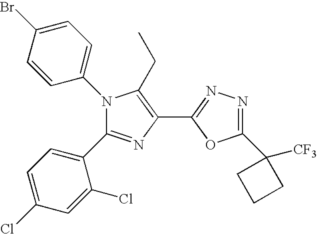

¹H NMR (400 MHz, CDCl₃) δ 7.54 (d, J=8.4 Hz, 2H), 7.35 (d, J=8.0 Hz, 1H), 7.32 (d, J=2.0 Hz, 1H), 7.26-7.23 (m, 1H), 7.07 (d, J=8.4 Hz, 2H), 2.99 (q, J=7.6 Hz, 2H), 2.88-2.74 (m, 4H), 2.20-2.11 (m, 2H), 1.12 (t, J=7.6 Hz, 3H),

MH+585.

### Example 51

### 2-(1-(4-Bromophenyl)-2-(2-chlorophenyl)-5-methyl-1H-imidazol-4-yl)-5-tert-butyl-1,3,4-oxadiazole

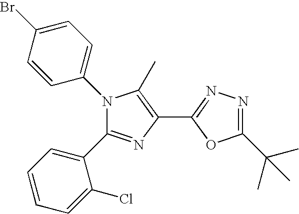

¹H NMR (400 MHz, CDCl₃) δ 7.50 (d, J=8.8 Hz, 2H), 7.43 (d, J=8.0 Hz, 1H), 7.32-7.27 (m, 2H), 7.26-7.24 (m, 1H), 7.04 (d, J=8.8 Hz, 2H), 2.54 (s, 3H), 1.50 (s, 9H).

MH+471.

### Example 52

### 2-(1-(4-Bromophenyl)-2-(2-chlorophenyl)-5-methyl-1H-imidazol-4-yl)-5-(1-(trifluoromethyl)cyclopropyl)-1,3,4-oxadiazole

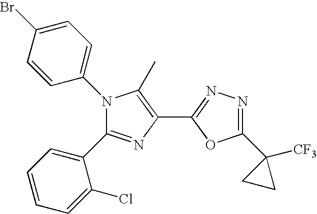

¹H NMR (400 MHz, CDCl₃) δ 7.51 (d, J=8.8 Hz, 2H), 7.43 (d, J=7.2 Hz, 1H), 7.35-7.30 (m, 2H), 7.28-7.24 (m, 1H), 7.04 (d, J=8.8 Hz, 2H), 2.54 (s, 3H), 1.64-1.60 (m, 4H).

MH+523.

### Example 53

### 2-(1-(4-Bromophenyl)-2-(2-chlorophenyl)-5-methyl-1H-imidazol-4-yl)-5-(1-(trifluoromethyl)cyclobutyl)-1,3,4-oxadiazole

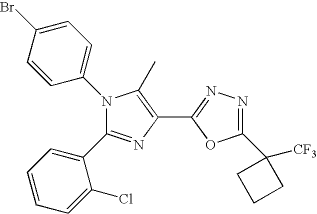

¹H NMR (400 MHz, CDCl₃) δ 7.51 (d, J=8.8 Hz, 2H), 7.43 (d, J=7.2 Hz, 1H), 7.34-7.29 (m, 2H), 7.28-7.24 (m, 1H), 7.05 (d, J=8.4 Hz, 2H), 2.89-2.74 (m, 4H), 2.55 (s, 3H), 2.19-2.12 (m, 2H).

MH+537.

### Example 54

### 2-(1-(4-Bromophenyl)-2-(2-chlorophenyl)-5-methyl-1H-imidazol-4-yl)-5-(1-(4-chlorophenyl)cyclopropyl)-1,3,4-oxadiazole

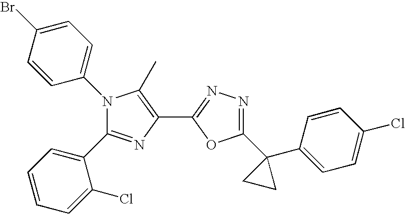

¹H NMR (400 MHz, CDCl₃) δ 7.49 (d, J=8.8 Hz, 2H), 7.43-7.40 (m, 3H), 7.33-7.29 (m, 3H), 7.27-7.23 (m, 2H), 7.02 (d, J=8.8 Hz, 2H), 2.48 (s, 3H), 1.79 (dd, J=7.2, 5.2 Hz, 2H), 1.42 (dd, J=7.2, 5.2 Hz, 2H).

MH+565.

### Example 55

### 2-(1-(4-Bromophenyl)-2-(2-chlorophenyl)-5-ethyl-1H-imidazol-4-yl)-5-tert-butyl-1,3,4-oxadiazole

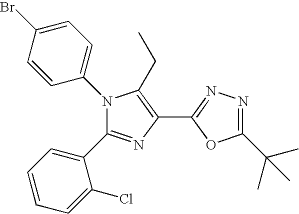

¹H NMR (400 MHz, CDCl₃) δ 7.50 (d, J=8.8 Hz, 2H), 7.42 (d, J=8.0 Hz, 1H), 7.30-7.28 (m, 2H), 7.26-7.22 (m, 1H), 7.08 (d, J=8.8 Hz, 2H), 3.00 (q, J=7.6 Hz, 2H), 1.50 (s, 9H), 1.11 (t, J=7.6 Hz, 3H).

MH+485.

### Example 56

### 2-(1-(4-Bromophenyl)-2-(2-chlorophenyl)-5-ethyl-1H-imidazol-4-yl)-5-(1-(trifluoromethyl)cyclopropyl)-1,3,4-oxadiazole

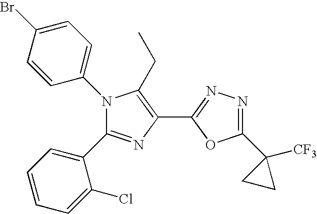

¹H NMR (400 MHz, CDCl₃) δ 7.51 (d, J=8.8 Hz, 2H), 7.41 (d, J=7.2 Hz, 1H), 7.31-7.28 (m, 2H), 7.25-7.23 (m, 1H), 7.08 (d, J=8.8 Hz, 2H), 2.98 (q, J=7.6 Hz, 2H), 1.63-1.59 (m, 4H), 1.11 (t, J=7.6 Hz, 3H).

MH+537.

### Example 57

### 2-(1-(4-Bromophenyl)-2-(2-chlorophenyl)-5-ethyl-1H-imidazol-4-yl)-5-(1-(trifluoromethyl)cyclobutyl)-1,3,4-oxadiazole

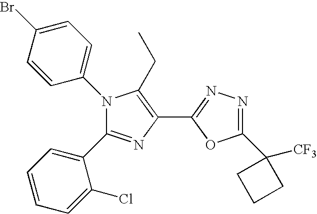

¹H NMR (400 MHz, CDCl₃) δ 7.51 (d, J=8.8 Hz, 2H), 7.41 (d, J=6.8 Hz, 1H), 7.31-7.28 (m, 2H), 7.25-7.22 (m, 1H), 7.09 (d, J=8.8 Hz, 2H), 3.00 (q, J=7.6 Hz, 2H), 2.89-2.74 (m, 4H), 2.19-2.11 (m, 2H), 1.13 (t, J=7.6 Hz, 3H).

MH+551.

### Example 58

### 2-(1-(4-Bromophenyl)-2-(2-chlorophenyl)-5-ethyl-1H-imidazol-4-yl)-5-(1-(4-chlorophenyl)cyclopropyl)-1,3,4-oxadiazole

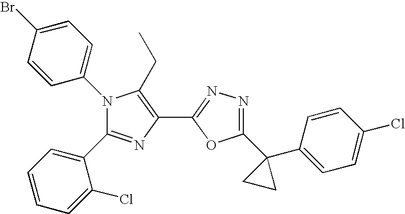

¹H NMR (400 MHz, CDCl₃) δ 7.49 (d, J=8.8 Hz, 2H), 7.43-7.37 (m, 3H), 7.33-7.28 (m, 4H), 7.25-7.21 (m, 1H), 7.06 (d, J=8.8 Hz, 2H), 2.93 (q, J=7.6 Hz, 2H), 1.79 (dd, J=7.2, 4.4 Hz, 2H), 1.42 (dd, J=7.2, 4.4 Hz, 2H), 1.05 (t, J=7.6 Hz, 3H).

MH+579.

### Example 59

### 2-tert-Butyl-5-(2-(2-chlorophenyl)-1-(4-chlorophenyl)-5-methyl-1H-imidazol-4-yl)-1,3,4-oxadiazole

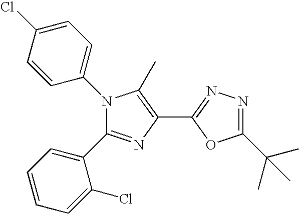

¹H NMR (400 MHz, CDCl₃) δ 7.43 (d, J=6.8 Hz, 1H), 7.34 (d, J=7.6 Hz, 2H), 7.32-7.24 (m, 3H), 7.11 (d, J=8.4 Hz, 2H), 2.54 (s, 3H), 1.50 (s, 9H).

MH+427.

### Example 60

### 2-(2-(2-Chlorophenyl)-1-(4-chlorophenyl)-5-methyl-1H-imidazol-4-yl)-5-(1-(trifluoromethyl)cyclopropyl)-1,3,4-oxadiazole

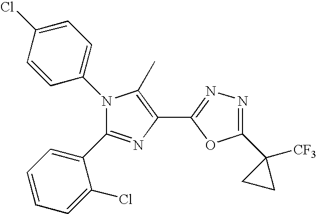

¹H NMR (400 MHz, CDCl₃) δ 7.42 (d, J=7.2 Hz, 1H), 7.35 (d, J=9.2 Hz, 2H), 7.32-7.30 (m, 2H), 7.28-7.24 (m, 1H), 7.11 (d, J=8.8 Hz, 2H), 2.54 (s, 3H), 1.63-1.59 (m, 4H).

MH+479.

### Example 61

### 2-(2-(2-Chlorophenyl)-1-(4-chlorophenyl)-5-methyl-1H-imidazol-4-yl)-5-(1-(trifluoromethyl)cyclobutyl)-1,3,4-oxadiazole

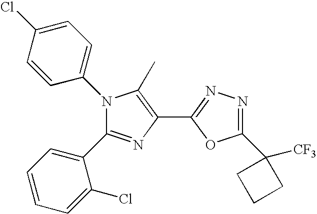

¹H NMR (400 MHz, CDCl₃) δ 7.43 (d, J=7.6 Hz, 1H), 7.35 (d, J=8.8 Hz, 2H), 7.32-7.27 (m, 2H), 7.25-7.24 (m, 1H), 7.11 (d, J=8.8 Hz, 2H), 2.89-2.74 (m, 4H), 2.55 (s, 3H), 2.19-2.11 (m, 2H).

MH+493.

### Example 62

### 2-(2-(2-Chlorophenyl)-1-(4-chlorophenyl)-5-methyl-1H-imidazol-4-yl)-5-(1-(4-chlorophenyl)cyclopropyl)-1,3,4-oxadiazole

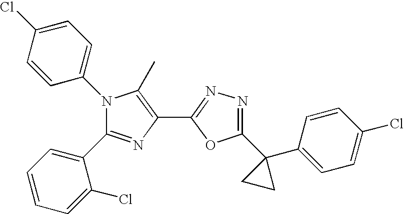

¹H NMR (400 MHz, CDCl₃) δ 7.43-7.39 (m, 3H), 7.35-7.28 (m, 6H), 7.27-7.22 (m, 1H), 7.08 (d, J=8.8 Hz, 2H), 2.48 (s, 3H), 1.78 (dd, J=7.2, 4.8 Hz, 2H), 1.42 (dd, J=7.2, 4.8 Hz, 2H).

MH+521.

### Example 63

### 2-tert-Butyl-5-(2-(2-chlorophenyl)-1-(4-chlorophenyl)-5-ethyl-1H-imidazol-4-yl)-1,3,4-oxadiazole

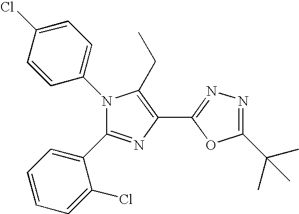

¹H NMR (400 MHz, CDCl₃) δ 7.41 (d, J=7.2 Hz, 1H), 7.34 (d, J=8.8 Hz, 2H), 7.30-7.27 (m, 2H), 7.25-7.22 (m, 1H), 7.15 (d, J=8.8 Hz, 2H), 3.00 (q, J=7.6 Hz, 2H), 1.50 (s, 9H), 1.11 (t, J=7.6 Hz, 3H).

MH+441.

### Example 64

### 2-(2-(2-Chlorophenyl)-1-(4-chlorophenyl)-5-ethyl-1H-imidazol-4-yl)-5-(1-(trifluoromethyl)cyclopropyl)-1,3,4-oxadiazole

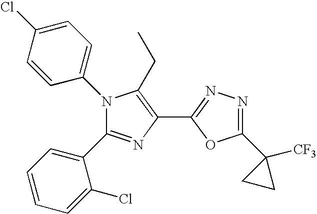

¹H NMR (400 MHz, CDCl₃) δ 7.41 (d, J=8.0 Hz, 1H), 7.35 (d, J=8.8 Hz, 2H), 7.31-7.28 (m, 2H), 7.25-7.22 (m, 1H), 7.15 (d, J=8.8 Hz, 2H), 2.98 (q, J=7.6 Hz, 2H), 1.66-1.59 (m, 4H), 1.11 (t, J=7.6 Hz, 3H).

MH+493.

### Example 65

### 2-(2-(2-Chlorophenyl)-1-(4-chlorophenyl)-5-ethyl-1H-imidazol-4-yl)-5-(1-(trifluoromethyl)cyclobutyl)-1,3,4-oxadiazole

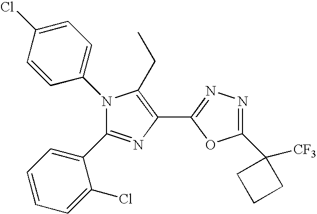

¹H NMR (400 MHz, CDCl₃) δ 7.41 (d, J=7.2 Hz, 1H), 7.35 (d, J=8.8 Hz, 2H), 7.30-7.28 (m, 2H), 7.25-7.22 (m, 1H), 7.15 (d, J=8.8 Hz, 2H), 3.00 (q, J=7.6 Hz, 2H), 2.89-2.74 (m, 4H), 2.19-2.11 (m, 2H), 1.13 (t, J=7.6 Hz, 3H).

MH+507.

### Example 66

### 2-(2-(2-Chlorophenyl)-1-(4-chlorophenyl)-5-ethyl-1H-imidazol-4-yl)-5-(1-(4-chlorophenyl)cyclopropyl)-1,3,4-oxadiazole

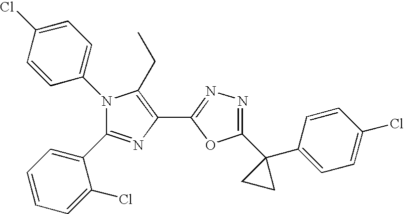

¹H NMR (400 MHz, CDCl₃) δ 7.42 (d, J=8.8 Hz, 2H), 7.38 (d, J=7.2 Hz, 1H), 7.35 (s, 1H), 7.32 (d, J=1.2 Hz, 2H), 7.30-7.28 (m, 3H), 7.25-7.21 (m, 1H), 7.12 (d, J=8.8 Hz, 2H), 2.93 (q, J=7.6 Hz, 2H), 1.79 (dd, J=7.2, 4.4 Hz, 2H), 1.42 (dd, J=7.2, 4.4 Hz, 2H), 1.06 (t, J=7.6 Hz, 3H).

MH+535.

### Example 67

### 2-(1-(4-Chlorophenyl)-2-(2,4-dichlorophenyl)-5-methyl-1H-imidazol-4-yl)-5-(1-(trifluoromethyl)cyclopropyl)-1,3,4-oxadiazole

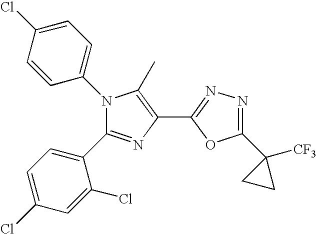

¹H NMR (400 MHz, CDCl₃) δ 7.40-7.35 (m, 3H), 7.33 (d, J=2.0 Hz, 2H), 7.27-7.24 (m, 1H), 7.09 (d, J=8.4 Hz, 2H), 2.53 (s, 3H), 1.63 (s, 2H), 1.61 (t, J=2.0 Hz,2H).

MH+513.

### Example 68

### 2-(1-(4-Chlorophenyl)-2-(2,4-dichlorophenyl)-5-methyl-1H-imidazol-4-yl)-5-(1-(trifluoromethyl)cyclobutyl)-1,3,4-oxadiazole

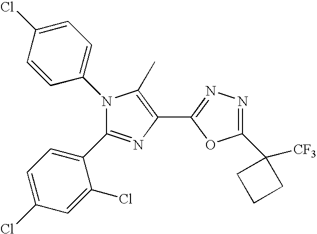

¹H NMR (400 MHz, CDCl₃) δ 7.39-7.36 (m, 3H), 7.32 (d, J=2.0 Hz, 1H), 7.27-7.24 (m, 1H), 7.09 (d, J=8.8 Hz, 2H), 2.88-2.74 (m, 4H), 2.54 (s, 3H), 2.20-2.12 (m, 2H).

MH+527.

### Example 69

### 2-(1-(4-Chlorophenyl)-2-(2,4-dichlorophenyl)-5-methyl-1H-imidazol-4-yl)-5-(1-(4-chlorophenyl)cyclopropyl)-1,3,4-oxadiazole

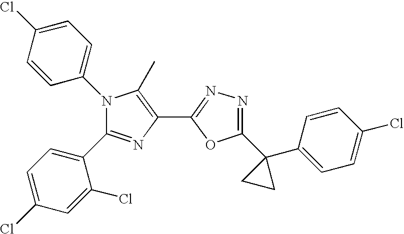

¹H NMR (400 MHz, CDCl₃) δ 7.41 (d, J=8.4 Hz, 2H), 7.36 (d, J=8.4 Hz, 2H), 7.33-7.30 (m, 4H), 7.24 (dd, J=8.4,2.0 Hz, 1H), 7.06 (d, J=8.8 Hz, 2H), 2.48 (s, 3H), 1.78 (dd, J=7.2, 4.8 Hz, 2H), 1.43 (dd, J=7.2, 4.8 Hz, 2H).

MH+555.

### Example 70

### 2-(1-(4-Chlorophenyl)-2-(2,4-dichlorophenyl)-5-ethyl-1H-imidazol-4-yl)-5-(1-(trifluoromethyl)cyclopropyl)-1,3,4-oxadiazole

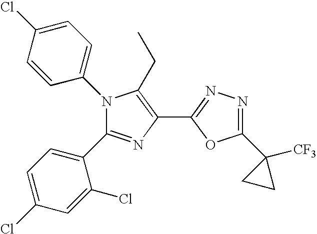

¹H NMR (400 MHz, CDCl₃) δ 7.38 (d, J 8.8 Hz, 2H), 7.34 (d, J=8.4 Hz, 1H), 7.32 (d, J=1.6 Hz, 1H), 7.24 (dd, J=8.4, 1.6 Hz, 1H), 7.13 (d, J=8.4 Hz, 2H), 2.97 (q, J=7.6 Hz, 2H), 1.63 (s, 2H), 1.61 (s, 2H), 1.11 (t, J=7.6 Hz, 3H).

MH+527.

### Example 71

### 2-(1-(4-Chlorophenyl)-2-(2,4-dichlorophenyl)-5-ethyl-1H-imidazol-4-yl)-5-(1-(trifluoromethyl)cyclobutyl)-1,3,4-oxadiazole

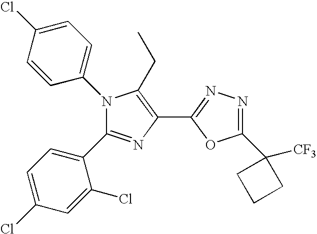

¹H NMR (400 MHz, CDCl₃) δ 7.38 (d, J=8.8 Hz, 2H), 7.35 (d, J=8.0 Hz, 1H), 7.32 (d, J=1.6 Hz, 1H), 7.24 (dd, J=8.0, 2.0 Hz, 1H), 7.13 (d, J=8.8 Hz, 2H), 2.99 (q, J=7.6 Hz, 2H), 2.88-2.74 (m, 4H), 2.20-2.12 (m, 2H), 1.12 (t, J=7.6 Hz, 3H).

MH+541.

### Example 72

### 2-(1-(4-Chlorophenyl)-2-(2,4-dichlorophenyl)-5-ethyl-1H-imidazol-4-yl)-5-(1-(4-chlorophenyl)cyclopropyl)-1,3,4-oxadiazole

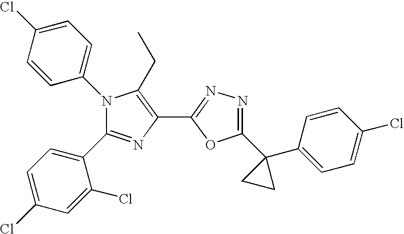

¹H NMR (400 MHz, CDCl₃) δ 7.42 (d, J=8.4 Hz, 2H), 7.38 (d, J=8.4 Hz, 2H), 7.35-7.30 (m, 4H), 7.23 (dd, J=8.4, 2.0 Hz, 1H), 7.10 (d, J=8.4 Hz, 2H), 2.91 (q, J=7.6 Hz, 2H), 1.79 (dd, J=7.2, 4.8 Hz, 2H), 1.43 (dd, J=7.2, 4.8 Hz, 2H), 1.05 (t, J=7.6 Hz, 3H).

MH+569.

### Example 73

### 2-(1-(4-Chlorophenyl)-2-(2,4-dichlorophenyl)-5-ethyl-1H-imidazol-4-yl)-5-(trifluoromethyl)-1,3,4-oxadiazole

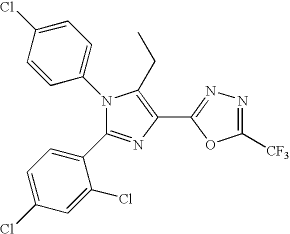

¹H NMR (400 MHz, CDCl₃) δ 7.40 (d, J=8.8 Hz, 2H), 7.34 (d, J=1.6 Hz, 1H), 7.32 (s, 1H), 7.26-7.24 (m, 1H), 7.15 (d, J=8.4 Hz, 2H), 3.03 (q, J=7.6 Hz, 2H), 1.14 (t, J=7.6 Hz, 3H).

MH+487.

### Example 74

### 2-(5-((1H-1,2,4-Triazol-1-yl)methyl)-1-(4-bromophenyl)-2-(2,4-dichlorophenyl)-1H-imidazol-4-yl)-5-tert-butyl-1,3,4-oxadiazole

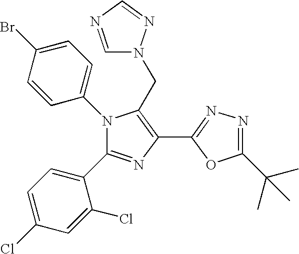

¹H NMR (400 MHz, CDCl₃) δ 8.46 (s, 1H), 7.83 (s, 1H), 7.54 (d, J=8.8 Hz, 2H), 7.35-7.32 (m, 2H), 7.26-7.24 (m, 1H), 7.19 (d, J=8.4 Hz, 2H), 5.68 (s, 2H), 1.50 (s, 9H).

MH+572.

### Example 75

### 2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclopropyl)-1,3,4-oxadiazole

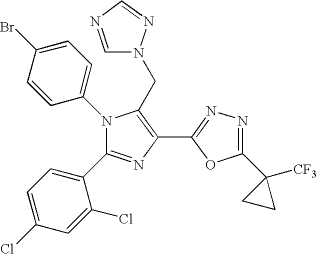

¹H NMR (400 MHz, CDCl₃) δ 8.40 (s, 1H), 7.84 (s, 1H), 7.54 (d, J=8.4 Hz, 2H), 7.36 (d, J=1.6 Hz, 1H), 7.33 (d, J=8.0 Hz, 1H), 7.27-7.25 (m, 1H), 7.20 (d, J=8.4 Hz, 2H), 5.66 (s, 2H), 1.64 (s, 4H).

MH+624.

### Example 76

### 2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclobutyl)-1,3,4-oxadiazole

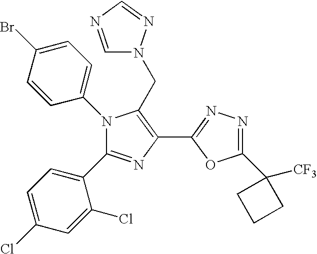

¹H NMR (400 MHz, CDCl₃) δ 8.42 (s, 1H), 7.85 (s, 1H), 7.54 (d, J=8.8 Hz, 2H), 7.35 (d, J=2.0 Hz, 1H), 7.33 (d, J=8.4 Hz, 1H), 7.27-7.24 (m, 1H), 7.19 (d, J=8.8 Hz, 2H), 5.68 (s, 2H), 2.84-2.78 (m, 4H), 2.20-2.14 (m, 2H).

MH+638.

### Example 77

### 2-(5-((1H-1,2,4-Triazol-1-yl)methyl)-1-(4-bromophenyl)-2-(2,4-dichlorophenyl)-1H-imidazol-4-yl)-5-(1-(4-chlorophenyl)cyclopropyl)-1,3,4-oxadiazole

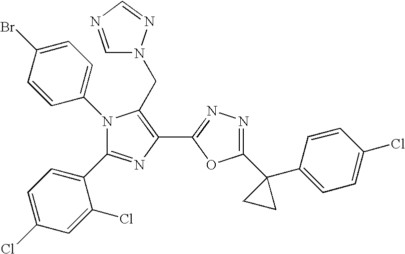

¹H NMR (400 MHz, CDCl₃) δ 8.39 (s, 1H), 7.82 (s, 1H), 7.53 (d, J=8.4 Hz, 2H), 7.42 (d, J=8.4 Hz, 2H), 7.34-7.30 (m, 4H), 7.26-7.23 (m, 1H), 7.18 (d, J=8.4 Hz, 2H), 5.61 (s, 2H), 1.80 (dd, J=6.8, 4.4 Hz, 2H), 1.47 (dd, J=6.8, 4.4 Hz, 2H).

MH+666.

### Example 78

### 2-(5-((1H-1,2,4-Triazol-1-yl)methyl)-1-(4-bromophenyl)-2-(2-chlorophenyl)-1H-imidazol-4-yl)-5-tert-butyl-1,3,4-oxadiazole

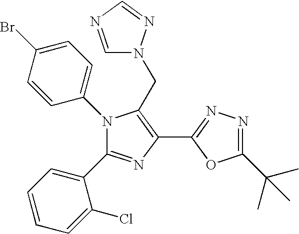

¹H NMR (400 MHz, CDCl₃) δ 8.46 (s, 1H), 7.84 (s, 1H), 7.51 (d, J=8.8 Hz, 2H), 7.39 (d, J=8.0 Hz, 1H), 7.34-7.31 (m, 2H), 7.28-7.24 (m, 1H), 7.19 (d, J=8.8 Hz, 2H), 5.69 (s, 2H), 1.50 (s, 9H).

MH+538.

### Example 79

### 2-(5-((1H-1,2,4-Triazol-1-yl)methyl)-1-(4-bromophenyl)-2-(2-chlorophenyl)-1H-imidazol-4-yl)-5-(1-(trifluoromethyl)cyclopropyl)-1,3,4-oxadiazole

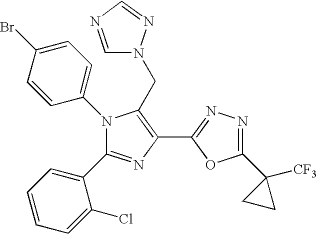

¹H NMR (400 MHz, CDCl₃) δ 8.39 (s, 1H), 7.84 (s, 1H), 7.51 (d, J=8.8 Hz, 2H), 7.38 (d, J=8.0 Hz, 1H), 7.35-7.31 (m, 2H), 7.28-7.24 (m, 1H), 7.19 (d, J=8.8 Hz, 2H), 5.67 (s, 2H), 1.64-1.60 (m, 4H).

MH+590.

### Example 80

### 2-(5-((1H-1,2,4-Triazol-1-yl)methyl)-1-(4-bromophenyl)-2-(2-chlorophenyl)-1H-imidazol-4-yl)-5-(1-(trifluoromethyl)cyclobutyl)-1,3,4-oxadiazole

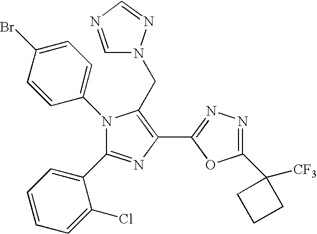

¹H NMR (400 MHz, CDCl₃) δ 8.39 (s, 1H), 7.84 (s, 1H), 7.51 (d, J=8.8 Hz, 2H), 7.39 (d, J=6.4 Hz, 1H), 7.34-7.31 (m, 2H), 7.28-7.24 (m, 1H), 7.20 (d, J=8.8 Hz, 2H), 5.70 (s, 2H), 2.88-2.75 (m, 4H), 2.21-2.12 (m, 2H).

MH+604.

### Example 81

### 2-(5-((1H-1,2,4-Triazol-1-yl)methyl)-1-(4-bromophenyl)-2-(2-chlorophenyl)-1H-imidazol-4-yl)-5-(1-(4-chlorophenyl)cyclopropyl)-1,3,4-oxadiazole

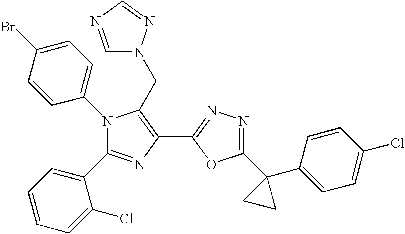

¹H NMR (400 MHz, CDCl₃) δ 8.38 (s, 1H), 7.82 (s, 1H), 7.50 (d, J=8.8 Hz, 2H), 7.42 (d, J=8.4 Hz, 2H), 7.37-7.30 (m, 5H), 7.27-7.23 (m, 1H), 7.18 (d, J=8.8 Hz, 2H), 5.63 (s, 2H), 1.80 (dd, J=7.2, 4.8 Hz, 2H), 1.46 (dd, J=7.2, 4.8 Hz, 2H).

MH+632.

### Example 82

### 2-(5-((1H-1,2,4-Triazol-1-yl)methyl)-2-(2-chlorophenyl)-1-(4-chlorophenyl)-1H-imidazol-4-yl)-5-tert-butyl-1,3,4-oxadiazole

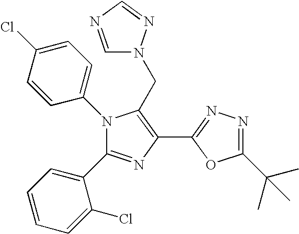

¹H NMR (400 MHz, CDCl₃) δ 8.46 (s, 1H), 7.84 (s, 1H), 7.41-7.31 (m, 6H), 7.28-7.24 (m, 2H), 5.69 (s, 2H), 1.50 (s, 9H).

MH+494.

### Example 83

### 2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclopropyl)-1,3,4-oxadiazole

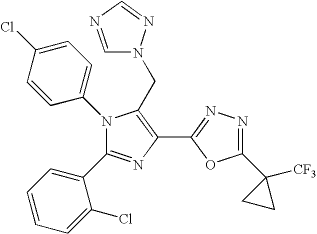

¹H NMR (400 MHz, CDCl₃) δ 8.40 (s, 1H), 7.85 (s, 1H), 7.40-7.33 (m, 6H), 7.29-7.25 (m, 2H), 5.68 (s, 2H), 1.64 (s, 4H).

MH+546.

### Example 84

### 2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclobutyl)-1,3,4-oxadiazole

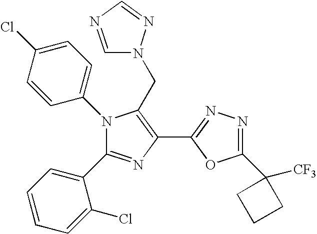

¹H NMR (400 MHz, CDCl₃) δ 8.42 (s, 1H), 7.85 (s, 1H), 7.40-7.32 (m, 6H), 7.28-7.25 (m, 2H), 5.70 (s, 2H), 2.88-2.75 (m, 4H), 2.191-2.12 (m, 2H).

MH+560.

### Example 85

### 2-(5-((1H-1,2,4-Triazol-1-yl)methyl)-2-(2-chlorophenyl)-1-(4-chlorophenyl)-1H-imidazol-4-yl)-5-(1-(4-chlorophenyl)cyclopropyl)-1,3,4-oxadiazole

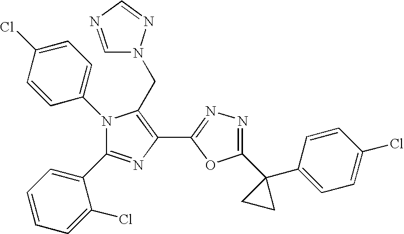

¹H NMR (400 MHz, CDCl₃) δ 8.38 (s, 1H), 7.82 (s, 1H), 7.42 (d, J=8.8 Hz, 2H), 7.38-7.30 (m, 8H), 7.27-7.23 (m, 2H), 5.63 (s, 2H), 1.80 (dd, J=7.2, 4.8 Hz, 2H), 1.46 (dd, J=7.2, 4.8 Hz, 2H).

MH+588.

### Example 86

### 2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclobutyl)-1,3,4-oxadiazole

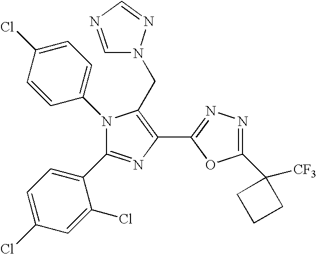

¹H NMR (400 MHz, CDCl₃) δ 8.42 (s, 1H), 7.85 (s, 1H), 7.39 (d, J=8.8 Hz, 2H), 7.35-7.34 (m, 1H), 7.32 (s, 1H), 7.27-7.24 (m, 3H), 5.69 (s, 2H), 2.84-2.78 (m, 4H), 2.21-2.14 (m, 2H).

MH+594.

### Example 87

### 2-(5-((1H-1,2,4-Triazol-1-yl)methyl)-1-(4-chlorophenyl)-2-(2,4-dichlorophenyl)-1H-imidazol-4-yl)-5-tert-butyl-1,3,4-oxadiazole

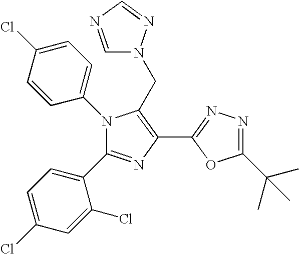

¹H NMR (400 MHz, CDCl₃) δ 9.35 (s, 1H), 8.24 (s, 1H), 7.42-7.35 (m, 4H), 7.28-7.25 (m, 3H), 5.77 (s, 2H), 1.49 (s, 9H).

MH+528.

### Example 88

### 2-(5-((1H-1,2,4-Triazol-1-yl)methyl)-1-(4-chlorophenyl)-2-(2,4-dichlorophenyl)-1H-imidazol-4-yl)-5-(1-(4-chlorophenyl)cyclopropyl)-1,3,4-oxadiazole

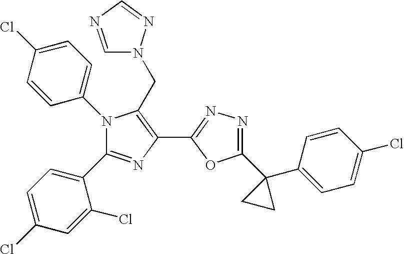

¹H NMR (400 MHz, CDCl₃) δ 9.31 (s, 1H), 8.13 (s, 1H), 7.51-7.24 (m, 11H), 5.60 (s, 2H), 1.79 (dd, J=7.2, 4.8 Hz, 2H), 1.46 (dd, J=7.2, 4.8 Hz, 2H).

MH+622.

### Example 89

### 2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclopropyl)-1,3,4-oxadiazole

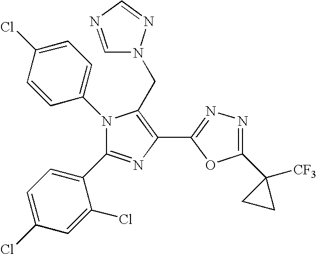

¹H NMR (400 MHz, CDCl₃) δ 9.76 (s, 1H), 8.41 (s, 1H), 7.45 (d, J=2.4 Hz, 2H), 7.40 (s, 4H), 7.35 (dd, J=8.4, 2.0 Hz, 1H), 5.71 (s, 2H), 1.65 (s, 4H).

MH+580.

### Preparation of 1,3,4-thiadiazole (formula (Ib))

### Example 90

### 2-tert-Butyl-5-(1-(4-chlorophenyl)-2-(2,4-dichlorophenyl)-5-methyl-1H-imidazol-4-yl)-1,3,4-thiadiazole

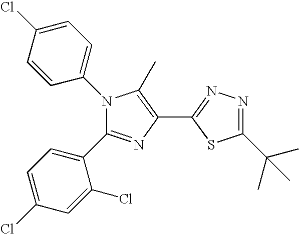

1-(4-chlorophenyl)-2-(2,4-dichlorophenyl)-5-methyl-N′-pivaloyl-1H-imidazole-4-carbohydrazide (40 mg, 0.083 mmol) was added to a microwave reactor containing Lawesson's reagent (101 mg, 0.25 mmol) in 1,4-dioxane (1 mL). The capped reactor was placed into a microwave reactor and the mixture was heated at 180° C. for 30 min. The reaction mixture was then purified by preparative HPLC to provide the title compound (38 mg, 0.080 mmol, 95%) as a yellow solid.

¹H NMR (400 MHz, CDCl₃) δ 7.39-7.35 (m, 3H), 7.32 (d, J=1.8 Hz, 1H), 7.23 (dd, J=8.2, 2.3 Hz, 1H), 7.13-7.09 (m, 2H), 2.58 (s, 3H), 1.52 (s, 9H).

MH+477.

The following compounds of Examples 91 to 179 were obtained by repeating the procedure of Example 90.

### Example 91

### 2-(1-(4-Chlorophenyl)-2-(2,4-dichlorophenyl)-5-methyl-1H-imidazol-4-yl)-5-cyclohexyl-1,3,4-thiadiazole

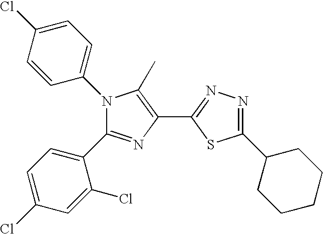

¹H NMR (400 MHz, CDCl₃) δ 7.38-7.32 (m, 4H), 7.24 (dd, J=8.2, 1.8 Hz, 1H), 7.11-7.07 (m, 2H), 3.20-3.14 (m, 1H), 2.58 (s, 3H), 2.21-2.17 (m, 2H), 1.89-1.84 (m, 2H), 1.77-1.74 (m, 1H), 1.65-1.56 (m, 1H), 1.50-1.40 (m, 2H), 1.36-1.25 (m, 2H).

MH+503.

### Example 92

### 2-(1-(4-Chlorophenyl)-2-(2,4-dichlorophenyl)-5-methyl-1H-imidazol-4-yl)-5-cyclopentyl-1,3,4-thiadiazole

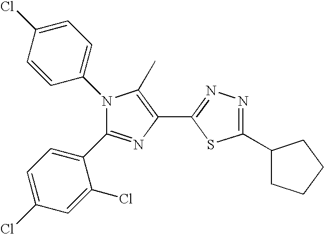

¹H NMR (400 MHz, CDCl₃) δ 7.38-7.32 (m, 4H), 7.24 (dd, J=8.2, 2.3 Hz, 1H), 7.11-7.07 (m, 2H), 3.63-3.55 (m, 1H), 2.58 (s, 3H), 2.28-2.22 (m, 2H), 1.94-1.81 (m, 4H), 1.75-1.71 (m, 2H).

MH+489.

### Example 93

### 2-(1-(4-Chlorophenyl)-2-(2,4-dichlorophenyl)-5-methyl-1H-imidazol-4-yl)-5-cyclobutyl-1,3,4-thiadiazole

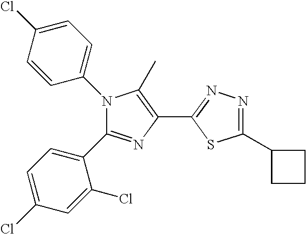

¹H NMR (400 MHz, CDCl₃) δ 7.39-7.33 (m, 4H), 7.24 (dd, J=8.7, 2.3 Hz, 1H), 7.11-7.08 (m, 2H), 4.03-3.97 (m, 1H), 2.59-2.51 (m, 5H), 2.47-2.38 (m, 2H), 2.15-2.01 (m, 2H).

MH+475.

### Example 94

### 2-(1-(4-Chlorophenyl)-2-(2,4-dichlorophenyl)-5-methyl-1H-imidazol-4-yl)-5-cycloheptyl-1,3,4-thiadiazole

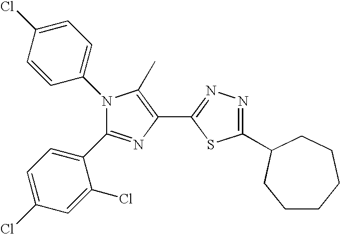

¹H NMR (400 MHz, CDCl₃) δ 7.38-7.32 (m, 4H), 7.24 (dd, J=8.7, 2.3 Hz, 1H), 7.11-7.07 (m, 2H), 3.43-3.36 (m, 1H), 2.58 (s, 3H), 2.23-2.16 (m, 2H), 1.89-1.80 (m, 4H), 1.70-1.59 (m, 6H).

MH+517.

### Example 95

### 2-(1-(4-Chlorophenyl)-2-(2,4-dichlorophenyl)-5-methyl-1H-imidazol-4-yl)-5-(hexan-2-yl)-1,3,4-thiadiazole

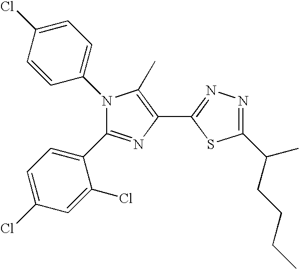

¹H NMR (400MHz, CDCl₃) δ 7.38-7.33 (m, 4H), 7.24 (d, J=1.8 Hz, 1H), 7.11-7.08 (m, 2H), 3.41-3.32 (m, 1H), 2.59 (s, 3H), 1.85-1.68 (m, 2H), 1.44 (d, J=6.9 Hz, 3H), 1.38-1.28 (m, 4H), 0.88 (t, J=7.3 Hz, 3H).

MH+505.

### Example 96

### 2-(1-(4-Chlorophenyl)-2-(2,4-dichlorophenyl)-5-methyl-1H-imidazol-4-yl)-5-(pentan-2-yl)-1,3,4-thiadiazole

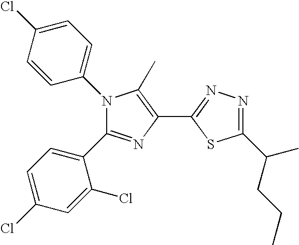

¹H NMR (400 MHz, CDCl₃) δ 7.39-7.33 (m, 4H), 7.24 (dd, J=8.2, 1.8 Hz, 1H), 7.11-7.08 (m, 2H), 3.43-3.34 (m, 1H), 2.59 (s, 3H), 1.84-1.66 (m, 2H), 1.45-1.33 (m, 5H), 0.92 (t, J=7.4 Hz, 3H).

MH+491.

### Example 97

### 2-sec-Butyl-5-(1-(4-chlorophenyl)-2-(2,4-dichlorophenyl)-5-methyl-1H-imidazol-4-yl)-1,3,4-thiadiazole

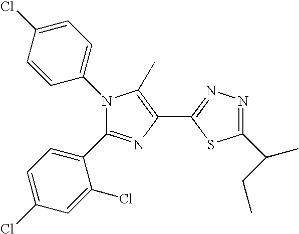

¹H NMR (400 MHz, CDCl₃) δ 7.39-7.33 (m, 4H), 7.24 (dd, J=8.2, 1.8 Hz, 1H), 7.11-7.08 (m, 2H), 3.32-3.25 (m, 1H), 2.59 (s, 3H), 1.88-1.74 (m, 2H), 1.44 (d, J=6.9 Hz, 3H), 0.97 (t, J=7.3 Hz, 3H).

MH+477.

### Example 98

### 2-(1-(4-Chlorophenyl)-2-(2,4-dichlorophenyl)-5-methyl-1H-imidazol-4-yl)-5-isopropyl-1,3,4-thiadiazole

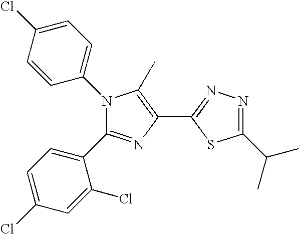

¹H NMR (400 MHz, CDCl₃) δ 7.39-7.35 (m, 3H), 7.33 (d, J=1.8 Hz, 1H), 7.24 (dd, J=8.2, 1.8 Hz, 1H), 7.11-7.08 (m, 2H), 3.52-3.45 (m, 1H), 2.58 (s, 3H), 1.47 (d, J=6.9 Hz, 6H).

MH+463.

### Example 99

### 2-(1-(4-Chlorophenyl)-2-(2,4-dichlorophenyl)-5-methyl-1H-imidazol-4-yl)-5-(pentan-3-yl)-1,3,4-thiadiazole

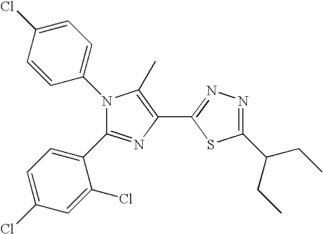

¹H NMR (400 MHz, CDCl₃) δ 7.38-7.32 (m, 4H), 7.27-7.23 (m, 1H), 7.11-7.08 (m, 2H), 3.11-3.09 (m, 1H), 2.60 (s, 3H), 1.91-1.68 (m, 4H), 0.93 (t, J=7.2 Hz, 6H).

MH+491.

### Example 100

### 2-(1-(4-Chlorophenyl)-2-(2,4-dichlorophenyl)-5-methyl-1H-imidazol-4-yl)-5-(1-phenylethyl)-1,3,4-thiadiazole

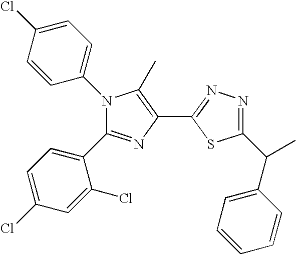

¹H NMR (400 MHz, CDCl₃) δ 7.37-7.33 (m, 5H), 7.31-7.28 (m, 4H), 7.22 (dd, J=8.2, 1.8 Hz, 1H), 7.09-7.05 (m, 2H), 4.62 (q, J=7.4 Hz, 1H), 2.58 (s, 3H), 1.87 (d, J=7.4 Hz, 3H).

MH+525.

### Example 101

### 2-(1-(4-Chlorophenyl)-2-(2,4-dichlorophenyl)-5-ethyl-1H-imidazol-4-yl)-5-cyclohexyl-1,3,4-thiadiazole

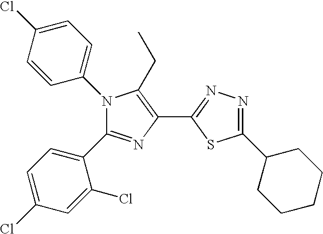

¹H NMR (400 MHz, CDCl₃) δ 7.39-7.35 (m, 211), 7.33-7.31 (m, 2H), 7.23 (dd, J=8.2, 2.3 Hz, 1H), 7.16-7.12 (m, 2H), 3.20-3.12 (m, 1H), 3.05 (q, J=7.4 Hz, 2H), 2.15 (d, J=11 Hz, 2H), 1.89-1.84 (m, 2H), 1.78-1.73 (m, 1H), 1.66-1.56 (m, 1H), 1.51-1.40 (m, 2H), 1.36-1.25 (m, 2H), 1.12 (t, J=7.3 Hz, 3H).

MH+517.

### Example 102

### 2-(1-(4-Chlorophenyl)-2-(2,4-dichlorophenyl)-5-ethyl-1H-imidazol-4-yl)-5-cyclopentyl-1,3,4-thiadiazole

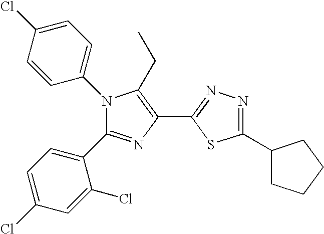

¹H NMR (400 MHz, CDCl₃) δ 7.39-7.35 (m, 2H), 7.33-7.31 (m, 2H), 7.23 (dd, J=8.2, 1.8 Hz, 1H), 7.15-7.12 (m, 2H), 3.62-3.55 (m, 11H), 3.05 (q, J=7.4 Hz, 2H), 2.28-2.22 (m, 2H), 1.95-1.81 (m, 4H), 1.75-1.72 (m, 2H), 1.12 (t, J=7.4 Hz, 3H).

MH+503.

### Example 103

### 2-(1-(4-Chlorophenyl)-2-(2,4-dichlorophenyl)-5-ethyl-1H-imidazol-4-yl)-5-cyclobutyl-1,3,4-thiadiazole

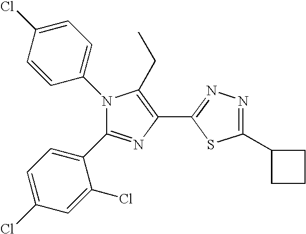

¹H NMR (400 MHz, CDCl₃) δ 7.38-7.35 (m, 2H), 7.34-7.32 (m, 2H), 7.23 (dd, J=8.2, 1.8 Hz, 1H), 7.16-7.12 (m, 2H), 4.03-3.95 (m, 1H), 3.05 (q, J=7.3 Hz, 2H), 2.59-2.51 (m, 2H), 2.48-2.38 (m, 2 H), 2.15-2.03 (m, 2H), 1.12 (t, J=7.3 Hz, 3H).

MH+489.

### Example 104

### 2-tert-Butyl-5-(1-(4-chlorophenyl)-2-(2,4-dichlorophenyl)-5-ethyl-1H-imidazol-4-yl)-1,3,4-thiadiazole

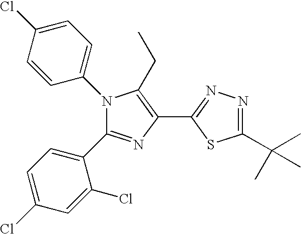

¹H NMR (300 MHz, CDCl₃) δ 7.39-7.31 (m, 4H), 7.23 (dd, J=8.2, 1.8 Hz, 1H), 7.15-7.13 (m, 2H), 3.05 (q, J=7.3 Hz, 2H), 1.52 (s, 9H), 1.13 (t, J=7.3 Hz, 3H).

MH+491.

### Example 105

### 2-(1-(4-Chlorophenyl)-2-(2,4-dichlorophenyl)-5-ethyl-1H-imidazol-4-yl)-5-(hexan-2-yl)-1,3,4-thiadiazole

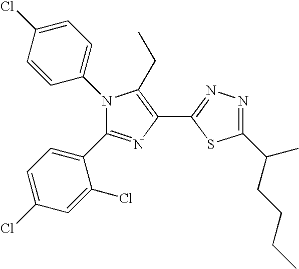

¹H NMR (300 MHz, CDCl₃) δ 7.39-7.31 (m, 4H), 7.23 (dd, J=8.2, 2.0 Hz, 1H), 7.16-7.12 (m, 2H), 3.39-3.32 (m, 1H), 3.06 (q, J=7.3 Hz, 2H), 1.83-1.64 (m, 2H), 1.44 (d, J=7.0 Hz, 3H), 1.35-1.32 (m, 4H), 1.12 (t, J=7.3 Hz, 3H), 0.88 (t, J=6.8 Hz,3H).

MH+519.

### Example 106

### 2-(1-(4-Chlorophenyl)-2-(2,4-dichlorophenyl)-5-ethyl-1H-imidazol-4-yl)-5-(pentan-2-yl)-1,3,4-thiadiazole

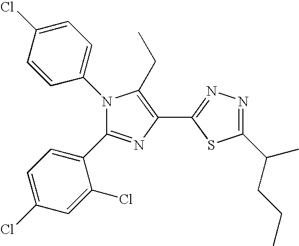

¹H NMR (400 MHz, CDCl₃) δ 7.38-7.31 (m, 4H), 7.26-7.21 (m, 1H), 7.15-7.12 (m, 2H), 3.41-3.36 (m, 1H), 3.05 (q, J=7.6 Hz, 2H), 1.83-1.65 (m, 2H), 1.44-1.35 (m, 5H), 1.12 (t, J=7.6 Hz, 3H), 0.93 (t, J=7.2 Hz, 3H).

MH+505.

### Example 107

### 2-sec-Butyl-5-(1-(4-chlorophenyl)-2-(2,4-dichlorophenyl)-5-ethyl-1H-imidazol-4-yl)-1,3,4-thiadiazole

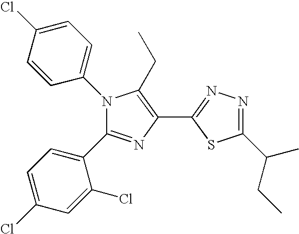

¹H NMR (400 MHz, CDCl₃) δ 7.39-7.32 (m, 4H), 7.25-7.22 (m, 1H), 7.16-7.13 (m, 2H), 3.32-3.27 (m, 1H), 3.06 (q, J=7.6 Hz, 2H), 1.89-1.74 (m, 2H), 1.44 (d, J=6.8 Hz, 3H), 1.12 (t, J=7.2 Hz, 3H), 0.98 (t, J=7.6 Hz, 3H).

MH+491

### Example 108

### 2-(1-(4-Bromophenyl)-2-(2,4-dichlorophenyl)-5-methyl-1H-imidazol-4-yl)-5-cyclohexyl-1,3,4-thiadiazole

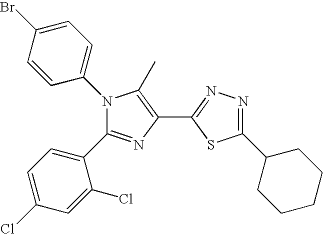

¹H NMR (400 MHz, CDCl₃) δ 7.52 (d, J=8.0 Hz, 2H), 7.34 (d, J=8.4 Hz, 2H), 7.26-7.23 (m, 1H), 7.03 (d, J=7.6 Hz, 2H), 3.21-3.14 (m, 1H), 2.59 (s, 3H), 2.21-2.18 (m, 2H), 1.89-1.85 (m, 2H), 1.77-1.74 (m, 1H), 1.65-1.55 (m, 2H), 1.50-1.40 (m, 2H), 1.36-1.25 (m, 1H).

MH+547.

### Example 109

### 2-(1-(4-Bromophenyl)-2-(2,4-dichlorophenyl)-5-methyl-1H-imidazol-4-yl)-5-tert-butyl-1,3,4-thiadiazole

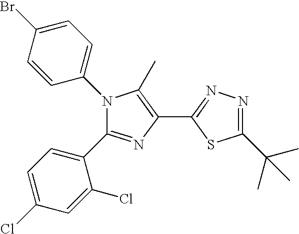

¹H NMR (400 MHz, CDCl₃) δ 7.54-7.51 (m, 2H), 7.35-7.33 (m, 2H), 7.25 (dd, J=8.2, 2.0 Hz, 1H), 7.06-7.02 (m, 2H), 2.59(s, 3H), 1.53 (s, 9H).

MH+521.

### Example 110

### 2-(1-(4-Bromophenyl)-2-(2,4-dichlorophenyl)-5-ethyl-1H-imidazol-4-yl)-5-tert-butyl-1,3,4-thiadiazole

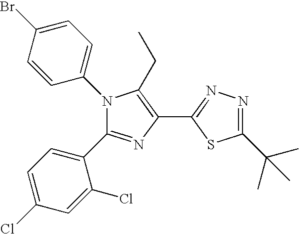

¹H NMR (400 MHz, CDCl₃) δ 7.54-7.51 (m, 2H), 7.34-7.32 (m, 2H), 7.23 (dd, J=8.4, 2.0 Hz, 1H), 7.09-7.06 (m, 2H), 3.04 (q, J=7.6 Hz, 2H), 1.52 (s, 9H), 1.13 (t, J=7.6 Hz, 3H).

MH+535.

### Example 111

### 2-(1-(4-Bromophenyl)-2-(2,4-dichlorophenyl)-5-methyl-1H-imidazol-4-yl)-5-cyclobutyl-1,3,4-thiadiazole

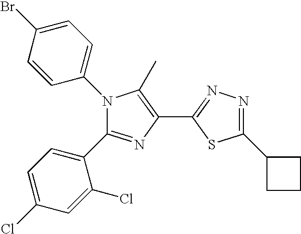

¹H NMR (400 MHz, CDCl₃) δ 7.53 (d, J=8.8 Hz, 2H), 7.36-7.33 (m, 2H), 7.26-7.24 (m, 1H), 7.04 (d, J=8.8 Hz, 2H), 4.04-3.96 (m, 1H), 2.60-2.54 (m, 5H), 2.48-2.438(m, 2H), 2.16-2.03 (m, 2H).

MH+519.

### Example 112

### 2-(1-(4-Bromophenyl)-2-(2,4-dichlorophenyl)-5-ethyl-1H-imidazol-4-yl)-5-cyclobutyl-1,3,4-thiadiazole

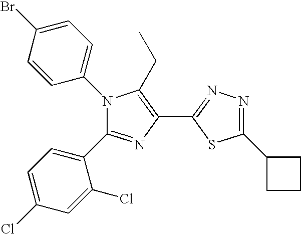

¹H NMR (400 MHz, CDCl₃) δ 7.53 (d, J=8.8 Hz, 2H), 7.35-7.32 (m, 2H), 7.24 (dd, J=8.4, 2.0 Hz, 1H), 7.08 (d, J=8.8 Hz, 2H), 4.04-3.95 (m, 1H), 3.05 (q, J=7.2 Hz, 2H), 2.59-2.51 (m, 2H), 2.46-2.39 (m, 2H), 2.16-2.00 (m, 2H), 1.12 (t, J=7.2 Hz, 3H).

MH+533.

### Example 113

### 2-tert-Butyl-5-(1-(4-chlorophenyl)-5-cyclopropyl-2-(2,4-dichlorophenyl)-1H-imidazol-4-yl)-1,3,4-thiadiazole

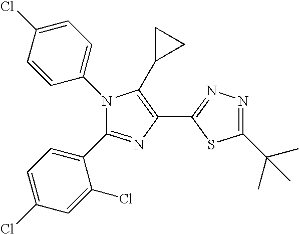

¹H NMR (400 MHz, CDCl₃) δ 7.36-7.32 (m, 4H), 7.25 (dd, J=8.4, 2.0 Hz, 1H), 7.13 (d, J=8.4 Hz, 2H), 1.92-1.86 (m, 1H), 1.53 (s, 9H), 0.94-0.89 (m, 2H), 0.69-0.65 (m, 2H).

MH+503.

### Example 114

### 2-(1-(4-Bromophenyl)-2-(2,4-dichlorophenyl)-5-isopropyl-1H-imidazo-4-yl)-5-tert-butyl-1,3,4-thiadiazole

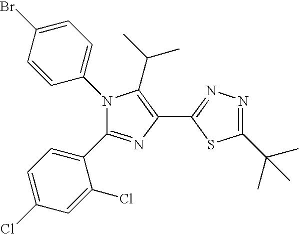

¹H NMR (400 MHz, CDCl₃) δ 7.51 (d, J=8.0 Hz, 2H), 7.32 (d, J=1.6 Hz, 1H), 7.29 (s, 1H), 7.20 (dd, J=11.2, 2.0 Hz, 1H), 7.09 (d, J=7.6 Hz, 2H), 3.44-3.44-3.40 (m, 1H), 1.52 (s, 9H), 1.38 (d, J=7.2 Hz, 6H).

MH+549.

### Example 115

### 2-(1-(4-Bromophenyl)-2-(2,4-dichlorophenyl)-5-isopropyl-1H-imidazol-4-yl)-5-cyclobutyl-1,3,4-thiadiazole

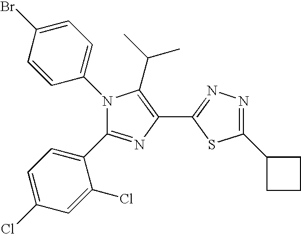

¹H NMR (400 MHz, CDCl₃) δ 7.51 (d, J=8.8 Hz, 2H), 7.32 (d, J=2.0 Hz, 1H), 7.28 (s, 1H), 7.20 (dd, J=8.4, 2.0 Hz, 1H), 7.08 (d, J=8.8 Hz, 2H), 4.02-3.96 (m, 1H), 3.46-3.39 (m, 1H), 2.59-2.40 (m, 4H), 2.15-2.02 (m, 2H), 1.37 (d, J=7.2 Hz, 6H).

MH+547.

### Example 116

### 2-(1-(4-Bromophenyl)-2-(2,4-dichlorophenyl)-5-isopropyl-1H-imidazol-4-yl)-5-(1-phenylcyclopropyl)-1,3,4-thiadiazole

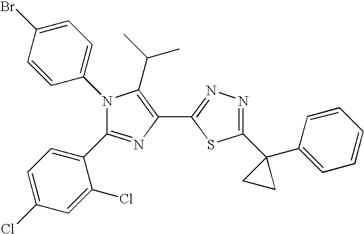

¹H NMR (400 MHz, CDCl₃) δ 7.51-7.48 (m, 4H), 7.36-7.27 (m, 4H), 7.22-7.20 (m, 1H), 7.16 (dd, J=8.4, 2.0 Hz, 1H), 7.05 (d, J=8.8 Hz, 2H), 3.41-3.34 (m, 1H), 1.92 (dd, J=7.2, 4.4 Hz, 2H), 1.51 (dd, J=6.8, 4.0 Hz, 2H), 1.35 (d, J=7.2 Hz, 6H).

MH+609.

### Example 117

### 2-(1-(4-Bromophenyl)-2-(2,4-dichlorophenyl)-5-ethyl-1H-imidazol-4-yl)-5-(1-phenylcyclopropyl)-1,3,4-thiadiazole

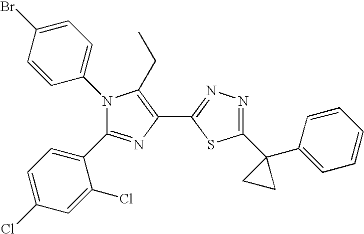

¹H NMR (400 MHz, CDCl₃) δ 7.52-7.49 (m, 4H), 7.37-7.25 (m, 5H), 7.19 (dd, J=8.4, 2.0 Hz, 1H), 7.04 (d, J=8.8 Hz, 2H), 3.01 (q, J=7.6 Hz, 2H), 1.92 (dd, J=6.8, 4.4 Hz, 2H), 1.51 (dd, J=6.8, 4.4 Hz, 2H), 1.10 (t, J=7.6 Hz, 3H).

MH+595.

### Example 118

### 2-(1-(4-Chlorophenyl)-2-(2,4-dichlorophenyl)-5-isopropyl-1H-imidazol-4-yl)-5-(1-phenylcyclopropyl)-1,3,4-thiadiazole

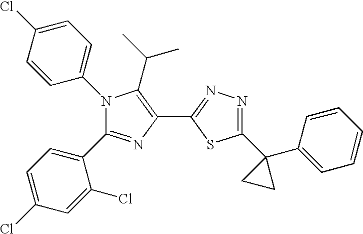

¹H NMR (400 MHz, CDCl₃) δ 7.48 (d, J=8.0 Hz, 2H), 7.35-7.27 (m, 6H), 7.20 (d, J=8.0 Hz, 1H), 7.16 (m, 1H), 7.11 (d, J=8.8 Hz, 2H), 3.40-3.36 (m, 1H), 1.92 (dd, J=6.4, 4.0 Hz, 2H), 1.50 (dd, J=6.8, 4.4 Hz, 2H), 1.34 (d, J=7.2 Hz, 6H).

MH+565.

### Example 119

### 2-(1-(4-Bromophenyl)-2-(2,4-dichlorophenyl)-5-propyl-1H-imidazol-4-yl)-5-tert-butyl-1,3,4-thiadiazole

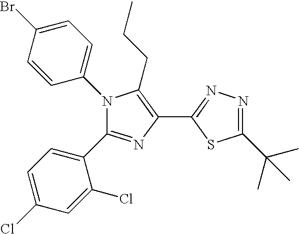

¹H NMR (400 MHz, CDCl₃) δ 7.51 (d, J=8.8 Hz, 2H), 7.32 (d, J=2.4 Hz, 1H), 7.30 (s, 1H), 7.22 (dd, J=8.0, 2.4 Hz, 1H), 7.05 (d, J=8.8 Hz, 2H), 2.99 (t, J=7.6 Hz, 2H), 1.59-1.51 (m, 11H), 0.85 (t, J=7.2 Hz, 3H).

MH+549.

### Example 120

### 2-(1-(4-Bromophenyl)-2-(2,4-dichlorophenyl)-5-propyl-1H-imidazol-4-yl)-5-cyclohexyl-1,3,4-thiadiazole

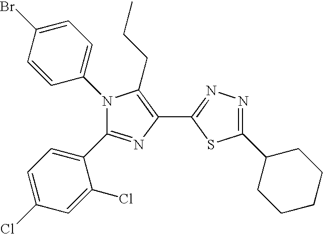

¹H NMR (400 MHz, CDCl₃) δ 7.51 (d, J=8.8 Hz, 2H), 7.32 (d, J=2.0 Hz, 1H), 7.30 (s, 1H), 7.22 (dd, J=8.0, 2.0 Hz, 1H), 7.05 (d, J=8.8 Hz, 2H), 3.20-3.14 (m, 1H), 2.99 (t, J=7.6 Hz, 2H), 2.19 (d, J=12.0 Hz, 2H), 1.88-1.72 (m, 4H), 1.62-1.25 (m, 6H), 0.84 (t, J=7.2 Hz, 3H).

MH+575.

### Example 121

### 2-(1-(4-Bromophenyl)-2-(2,4-dichlorophenyl)-5-propyl-1H-imidazol-4-yl)-5-(1-phenylcyclopropyl)-1,3,4-thiadiazole

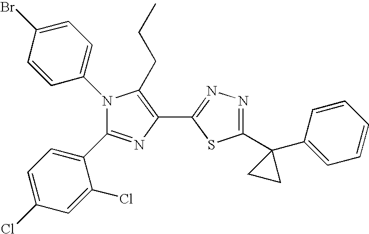

¹H NMR (400 MHz, CDCl₃) δ 7.51-7.47 (m, 4H), 7.35-7.28 (m, 4H), 7.26-7.23 (m, 1H), 7.18 (dd, J=8.4,2.0 Hz, 1H), 7.02 (d, J=8.8 Hz, 2H), 2.96 (t, J=8.0 Hz, 2H), 1.92 (dd, J=6.8, 4.4 Hz, 2H), 1.51 (dd, J=7.2, 4.4 Hz, 2H), 0.83 (t, J=7.2 Hz, 3H).

MH+609.

### Example 122

### 2-(1-(4-Bromophenyl)-2-(2,4-dichlorophenyl)-5-propyl-1H-imidazol-4-yl)-5-cyclobutyl-1,3,4-thiadiazole

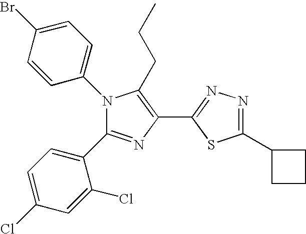

¹H NMR (400 MHz, CDCl₃) δ 7.52 (d, J=8.8 Hz, 2H), 7.33 (d, J=2.0 Hz, 1H), 7.31 (s, 1H), 7.23 (dd, J=8.0, 2.0 Hz, 1H), 7.05 (d, J=8.8 Hz, 2H), 4.03-3.99 (m, 1H), 3.00 (t, J=7.6 Hz, 2H), 2.57-2.51 (m, 2H), 2.46-2.41 (m, 2H), 2.15-2.01 (m, 2H), 1.56-1.50 (m, 2H), 0.84 (t, J=7.2 Hz, 3H).

MH+547.

### Example 123

### 2-(1-(4-Bromophenyl)-2-(2,4-dichlorophenyl)-5-propyl-1H-imidazol-4-yl)-5-(1-(4-chlorophenyl)cyclopropyl)-1,3,4-thiadiazole

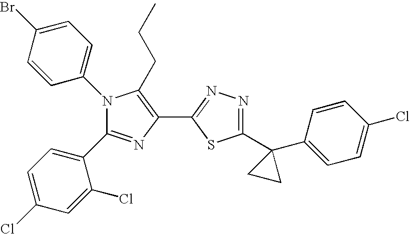

¹H NMR (400 MHz, CDCl₃) δ 7.50 (d, J=8.8 Hz, 2H), 7.42 (d, J=8.8 Hz, 2H), 7.33-7.29 (m, 3H), 7.26-7.23 (m, 1H), 7.18 (dd, J=8.4, 2.0 Hz, 1H), 7.02 (d, J=8.8 Hz, 2H), 2.95 (t, J=8.0 Hz, 2H), 1.90 (dd, J=7.2, 4.4 Hz, 2H), 1.54-1.46 (m, 4H), 0.83 (t, J=7.2 Hz, 3H).

MH+643.

### Example 124

### 2-(1-(4-Bromophenyl)-2-(2,4-dichlorophenyl)-5-propyl-1H-imidazol-4-yl)-5-(1-(4-chlorophenyl)cyclobutyl)-1,3,4-thiadiazole

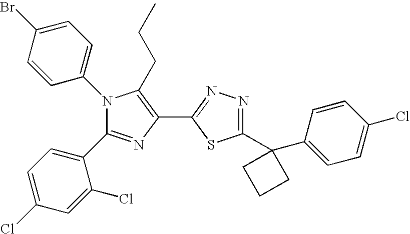

¹H NMR (400 MHz, CDCl₃) δ 7.47 (d, J=6.8 Hz, 2H), 7.26-7.18 (m, 6H), 7.16 (dd, J=6.8, 3.2 Hz, 1H), 6.99 (d, J=7.2 Hz, 2H), 3.05-2.93 (m, 4H), 2.80-2.73 (m, 2H), 2.31-2.29 (m, 1H), 2.00-1.98 (m, 1H), 1.51-1.48 (m, 2H), 0.81 (t, J=7.2 Hz, 3H).

MH+657.

### Example 125

### 2-tert-Butyl-5-(1-(4-chlorophenyl)-2-(2,4-dichlorophenyl)-5-propyl-1H-imidazol-4-yl)-1,3,4-thiadiazole

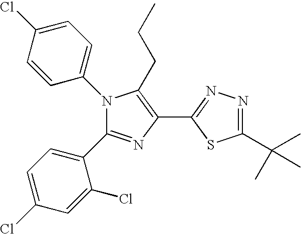

¹H NMR (400 MHz, CDCl₃) δ 7.35 (d, J=8.8 Hz, 2H), 7.32-7.31 (m, 1H), 7.29 (s, 1H), 7.11 (dd, J=8.4, 2.0 Hz, 1H), 7.06 (d, J=8.8 Hz, 2H), 2.99 (t, J=8.0 Hz, 2H), 1.57-1.41 (m, 11H), 0.84 (t, J=7.2 Hz, 3H).

MH+505.

### Example 126

### 2-(1-(4-Chlorophenyl)-2-(2,4-dichlorophenyl)-5-propyl-1H-imidazol-4-yl)-5-cyclobutyl-1,3,4-thiadiazole

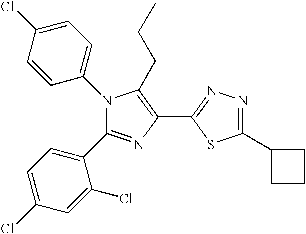

¹H NMR (400 MHz, CDCl₃) δ 7.36 (d, J=8.4 Hz, 2H), 7.32 (d, J=2.4 Hz, 1H), 7.30 (s, 1H), 7.22 (dd, J=8.0, 2.0 Hz, 1H), 7.11 (d, J=8.4 Hz, 2H), 4.05-3.96 (m, 1H), 2.99 (t, J=8.0 Hz, 2H), 2.59-2.51 (m, 2H), 2.48-2.38 (m, 2H), 2.17-2.02 (m, 2H), 1.57-1.48 (m, 2H), 0.84 (t, J=7.2 Hz, 3H).

MH+503.

### Example 127

### 2-(1-(4-Chlorophenyl)-2-(2,4-dichlorophenyl)-5-propyl-1H-imidazol-4-yl)-5-(1-(4-chlorophenyl)cyclopropyl)-1,3,4-thiadiazole

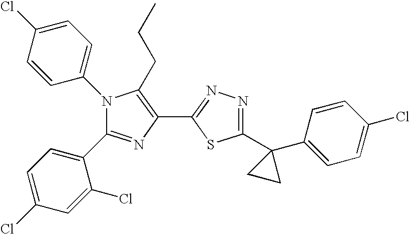

¹H NMR (400 MHz, CDCl₃) δ 7.42 (d, J=8.8 Hz, 2H), 7.35-7.28 (m, 5H), 7.25-7.23 (m, 1H), 7.18 (dd, J=8.4,2.0 Hz, 1H), 7.08 (d, J=8.8 Hz, 2H), 2.95 (t, J=8.0 Hz, 2H), 1.89 (dd, J=7.2, 4.4 Hz, 2H), 1.53-1.46 (m, 4H), 0.82 (t, J=7.2 Hz, 3H).

MH+599.

### Example 128

### 2-(1-(4-Chlorophenyl)-2-(2,4-dichlorophenyl)-5-propyl-1H-imidazol-4-yl)-5-(1-(4-chlorophenyl)cyclobutyl)-1,3,4-thiadiazole

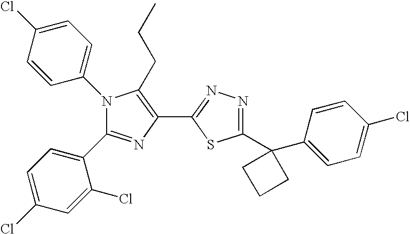

¹H NMR (400 MHz, CDCl₃) δ 7.30 (d, J=8.4 Hz, 2H), 7.27-7.20 (m, 6H), 7.15 (d, J=8.4 Hz, 1H), 7.05 (d, J=8.4 Hz, 2H), 3.01-2.98 (m, 2H), 2.94 (t, J=8.0 Hz, 2H), 2.79-2.72 (m, 2H), 2.33-2.26 (m, 1H), 1.99-1.94 (m, 1H), 1.54-1.44 (m, 2H), 0.80 (t, J=7.2 Hz, 3H).

MH+613.

### Example 129

### 2-(1-(4-Chlorophenyl)-2-(2,4-dichlorophenyl)-5-propyl-1H-imidazol-4-yl)-5-cyclohexyl-1,3,4-thiadiazole

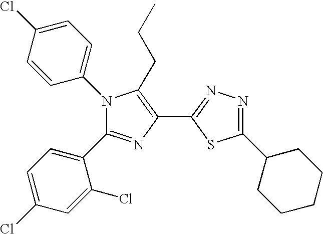

¹H NMR (400 MHz, CDCl₃) δ 7.37 (d, J 8.8 Hz, 2H), 7.34-7.32 (m, 2H), 7.23 (dd, J=8.4, 2.0 Hz, 1H), 7.12 (d, J=8.8 Hz, 2H), 3.25-3.17 (m, 1H), 2.99 (t, J=8.0 Hz, 2H), 2.20 (d, J=10.0 Hz, 2H), 1.88-1.84 (m, 2H), 1.76-1.73 (m, 2H), 1.65-1.40 (m, 6H), 1.35-1.24 (m, 2H), 0.84 (t, J=7.2 Hz, 3H).

MH+531.

### Example 130

### 2-(1-(4-Bromophenyl)-2-(2,4-dichlorophenyl)-5-ethyl-1H-imidazol-4-yl)-5-(1-(4-chlorophenyl)cyclopropyl)-1,3,4-thiadiazole

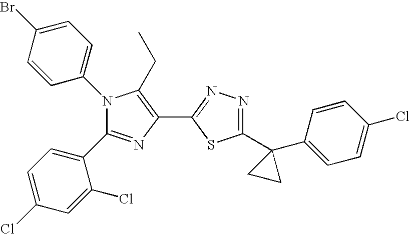

¹H NMR (400 MHz, CDCl₃) δ 7.51 (d, J=8.8 Hz, 2H), 7.43 (d, J=8.8 Hz, 2H), 7.33 (s, 1H), 7.30 (dd, J=6.8, 2.0 Hz, 2H), 7.27-7.25 (m, 1H), 7.19 (dd, J=8.0, 2.0 Hz, 1H), 7.04 (d, J=8.4 Hz, 2H), 3.01 (q, J=7.6 Hz, 2H), 1.90 (dd, J=7.2, 4.4 Hz, 2H), 1.48 (dd, J=7.2, 4.4 Hz, 2H), 1.10 (t, J=7.2 Hz, 3H).

MH+629.

### Example 131

### 2-(1-(4-Bromophenyl)-2-(2,4-dichlorophenyl)-5-ethyl-1H-imidazol-4-yl)-5-(1-(4-chlorophenyl)cyclobutyl)-1,3,4-thiadiazole

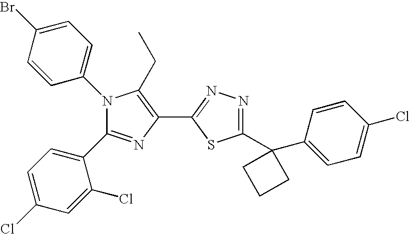

¹H NMR (400 MHz, CDCl₃) δ 7.51 (d, J=8.0 Hz, 2H), 7.32-7.26 (m, 6H), 7.21-7.19 (m, 1H), 7.05 (d, J=7.6 Hz, 2H), 3.09-3.01 (m, 4H), 2.81 (q, J=8.8 Hz, 2H), 2.39-2.32 (m, 1H), 2.05-1.98 (m, 1H), 1.12 (t, J=7.2 Hz, 3H).

MH+643.

### Example 132

### 2-(1-(4-Bromophenyl)-2-(2,4-dichlorophenyl)-5-ethyl-1H-imidazol-4-yl)-5-(1-(4-methoxyphenyl)cyclopropyl)-1,3,4-thiadiazole

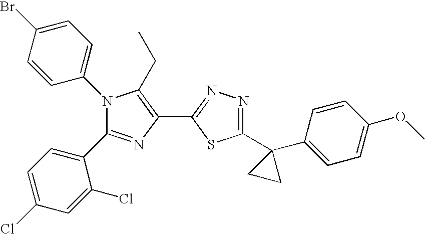

¹H NMR (400 MHz, CDCl₃) δ 7.50 (d, J=8.8 Hz, 2H), 7.41 (d, J=8.8 Hz, 2H), 7.29-7.24 (m, 2H), 7.19 (dd, J=8.0, 2.0 Hz, 1H), 7.04 (d, J=8.4 Hz, 2H), 6.86 (d, J=8.8 Hz, 2H), 3.79 (s, 3H), 3.00 (q, J=7.6 Hz, 2H), 1.90 (dd, J=6.8, 4.0 Hz, 2H), 1.48 (dd, J=6.8, 4.0 Hz, 2H), 1.09 (t, J=7.6 Hz, 3H).

MH+625.

### Example 133

### 2-(1-(4-Bromophenyl)-2-(2,4-dichlorophenyl)-5-ethyl-1H-imidazol-4-yl)-5-(1-p-tolylcyclopropyl)-1,3,4-thiadiazole

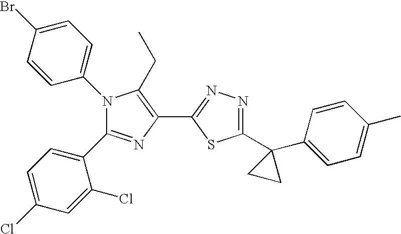

¹H NMR (400 MHz, CDCl₃) δ 7.50 (d, J 8.8 Hz, 2H), 7.37 (d, J=8.0 Hz, 2H), 7.28 (d, J=2.0 Hz, 1H), 7.27-7.24 (m, 1H), 7.19 (dd, J8.0, 2.0 Hz, 1H), 7.14 (d, J=7.6 Hz, 2H), 7.04 (d, J=8.8 Hz, 2H), 3.00 (q, J=7.6 Hz, 2H), 2.33 (s, 3H), 1.91 (dd, J=6.8, 4.0 Hz, 2H), 1.49 (dd, J=7.2, 4.0 Hz, 2H), 1.09 (t, J=7.6 Hz, 3H).

MH+609.

### Example 134

### 2-(1-(4-Bromophenyl)-2-(2,4-dichlorophenyl)-5-ethyl-1H-imidazol-4-yl)-5-(1-(2,4-dichlorophenyl)cyclopropyl)-1,3,4-thiadiazole

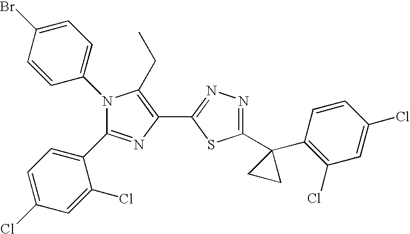

¹H NMR (400 MHz, CDCl₃) δ 7.51 (d, J=8.4 Hz, 2H), 7.45 (d, J=8.4 Hz, 1H), 7.42 (d, J=2.0 Hz, 1H), 7.30-7.25 (m, 3H), 7.19 (dd, J=8.0, 2.0 Hz, 1H), 7.04 (d, J=8.8 Hz, 2H), 3.00 (q, J=7.2 Hz, 2H), 2.02 (dd, J=7.2, 4.8 Hz, 2H), 1.53 (dd, J=7.2, 4.8 Hz, 2H), 1.09 (t, J=7.2 Hz, 3H).

MH+663.

### Example 135

### 2-(1-(4-Bromophenyl)-2-(2,4-dichlorophenyl)-5-ethyl-1H-imidazol-4-yl)-5-(1-(4-chlorophenyl)cyclopentyl)-1,3,4-thiadiazole

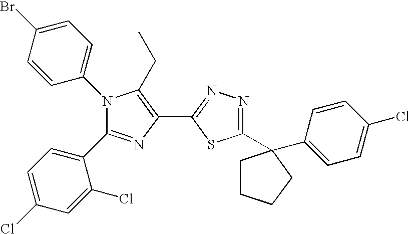

¹H NMR (400 MHz, CDCl₃) δ 7.51 (d, J=8.8 Hz, 2H), 7.37 (d, J=8.8 Hz, 2H), 7.30 (d, J=2.4 Hz, 1H), 7.28-7.24 (m, 3H), 7.20 (dd, J=8.4, 2.0 Hz, 1H), 7.03 (d, J=8.4 Hz, 2H), 3.02 (q, J=7.6 Hz, 2H), 2.83-2.79 (m, 2H), 2.27-2.24 (m, 2H), 1.87-1.83 (m, 4H), 1.10 (t, J=7.2 Hz, 3H).

MH+657.

### Example 136

### 2-(1-(4-Bromophenyl)-2-(2,4-dichlorophenyl)-5-methyl-1H-imidazol-4-yl)-5-(1-(trifluoromethyl)cyclopropyl)-1,3,4-thiadiazole

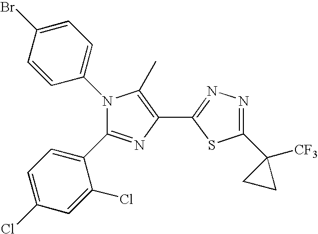

¹H NMR (400 MHz, CDCl₃) δ 7.54 (d, J=8.4 Hz, 2H), 7.35-7.33 (m, 1H), 7.27-7.25 (m, 2H), 7.04 (d, J=8.8 Hz, 2H), 2.59 (s, 3H), 1.70 (s, 2H), 1.63-160 (m, 2H).

MH+573.

### Example 137

### 2-(1-(4-Bromophenyl)-2-(2,4-dichlorophenyl)-5-methyl-1H-imidazol-4-yl)-5-(1-(trifluoromethyl)cyclobutyl)-1,3,4-thiadiazole

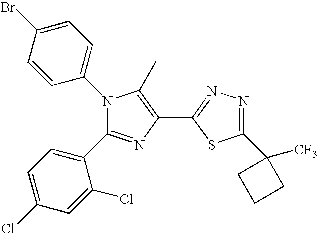

¹H NMR (400 MHz, CDCl₃) δ 7.53 (d, J=8.8 Hz, 2H), 7.34 (d, J=2.4 Hz, 1H), 7.32 (s, 1H), 7.26-7.24 (m, 1H), 7.04 (d, J=8.4 Hz, 2H), 2.82 (t, J=8.8 Hz, 4H), 2.61 (s, 3H), 2.20-2.12 (m, 2H).

MH+587.

### Example 138

### 2-(1-(4-Bromophenyl)-2-(2,4-dichlorophenyl)-5-methyl-1H-imidazol-4-yl)-5-(1-(4-chlorophenyl)cyclopropyl)-1,3,4-thiadiazole

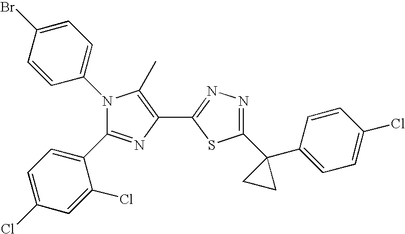

¹H NMR (400 MHz, CDCl₃) δ 7.51 (d, J=8.8 Hz, 2H), 7.43 (d, J=8.8 Hz, 2H), 7.33-7.28 (m, 3H), 7.26-7.23 (m, 1H), 7.21 (dd, J=8.4, 2.0 Hz, 1H), 7.00 (d, J=8.4 Hz, 2H), 2.55 (s, 3H), 1.90 (dd, J=6.8, 4.4 Hz, 2H), 1.49 (dd, J=6.8, 4.4 Hz, 2H).

MH+615.

### Example 139

### 2-(1-(4-Bromophenyl)-2-(2,4-dichlorophenyl)-5-ethyl-1H-imidazol-4-yl)-5-(1-(trifluoromethyl)cyclopropyl)-1,3,4-thiadiazole

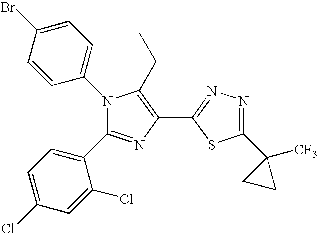

¹H NMR (400 MHz, CDCl₃) δ 7.53 (d, J=8.4 Hz, 2H), 7.34 (d, J=2.0 Hz, 2H), 7.31 (s, 1H), 7.24 (dd, J=8.4, 2.0 Hz, 1H), 7.08 (d, J=8.8 Hz, 2H), 3.04 (q, J=7.6 Hz, 2H), 1.71-1.69 (m, 2H), 1.62-1.59 (m, 2H), 1.13 (t, J=7.6 Hz, 3H).

MH+587.

### Example 140

### 2-(1-(4-Bromophenyl)-2-(2,4-dichlorophenyl)-5-ethyl-1H-imidazol-4-yl)-5-(1-(trifluoromethyl)cyclobutyl)-1,3,4-thiadiazole

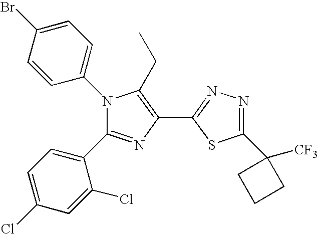

¹H NMR (400 MHz, CDCl₃) δ 7.54 (d, J=8.8 Hz, 2H), 7.33 (d, J=2.0 Hz, 1H), 7.31 (s, 1H), 7.24 (dd, J=8.4, 2.0 Hz, 1H), 7.08 (d, J=8.4 Hz, 2H), 3.07 (q, J=7.6 Hz, 2H), 2.82 (t, J=8.0 Hz, 4H), 2.20-2.12 (m, 2H), 1.15 (t, J=7.6 Hz, 3H).

MH+601.

### Example 141

### 2-(1-(4-Bromophenyl)-2-(2-chlorophenyl)-5-methyl-1H-imidazol-4-yl)-5-tert-butyl-1,3,4-thiadiazole

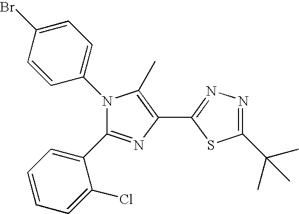

¹H NMR (400 MHz, CDCl₃) δ 7.49 (d, J=8.8 Hz, 2H), 7.40 (d, J=7.2 Hz, 1H), 7.31-7.30 (m, 2H), 7.27-7.23 (m, 1H), 7.05 (d, J=8.8 Hz, 2H), 2.60 (s, 3H), 1.52 (s, 9H).

MH+487.

### Example 142

### 2-(1-(4-Bromophenyl)-2-(2-chlorophenyl)-5-methyl-1H-imidazol-4-yl)-5-(1-(trifluoromethyl)cyclopropyl)-1,3,4-thiadiazole

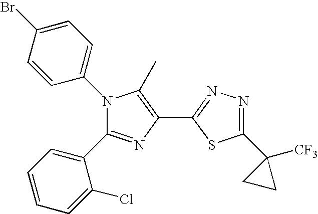

¹H NMR (400 MHz, CDCl₃) δ 7.50 (d, J=8.8 Hz, 2H), 7.40 (d, J=8.0 Hz, 1H), 7.33-7.29 (m, 2H), 7.28-7.24 (m, 1H), 7.05 (d, J=8.8 Hz, 2H), 2.59 (s, 3H), 1.72-1.59 (m, 4H).

MH+539.

### Example 143

### 2-(1-(4-Bromophenyl)-2-(2-chlorophenyl)-5-methyl-1H-imidazol-4-yl)-5-(1-(trifluoromethyl)cyclobutyl)-1,3,4-thiadiazole

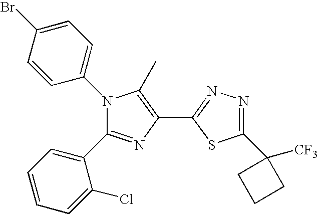

¹H NMR (400 MHz, CDCl₃) δ 7.51 (d, J=8.8 Hz, 2H), 7.40 (d, J=7.2 Hz, 1H), 7.32-7.29 (m, 2H), 7.28-7.24 (m, 1H), 7.06 (d, J=8.8 Hz, 2H), 2.82 (t, J=Hz, 4H), 2.62 (s, 3H), 2.20-2.12 (m, 2H).

MH+553.

### Example 144

### 2-(1-(4-Bromophenyl)-2-(2-chlorophenyl)-5-methyl-1H-imidazol-4-yl)-5-(1-(4-chlorophenyl)cyclopropyl)-1,3,4-thiadiazole

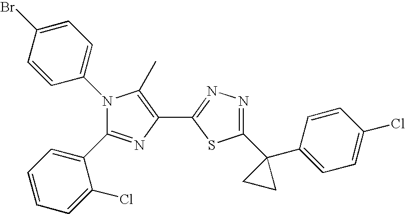

¹H NMR (400 MHz, CDCl₃) δ 7.48 (d, J=8.8 Hz, 2H), 7.43 (d, J=8.8 Hz, 2H), 7.35-7.30 (m, 3H), 7.29-7.25 (m, 2H), 7.24-7.20 (m, 1H), 7.02 (d, J=8.8 Hz, 2H), 2.56 (s, 3H), 1.90 (dd, J=7.2, 4.0 Hz, 2H), 1.49 (dd, J=7.2, 4.0 Hz, 2H).

MH+581.

### Example 145

### 2-(1-(4-Bromophenyl)-2-(2-chlorophenyl)-5-ethyl-1H-imidazol-4-yl)-5-tert-butyl-1,3,4-thiadiazole

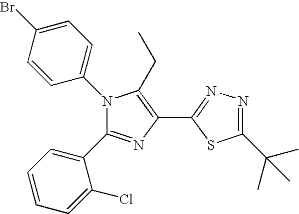

¹H NMR (400 MHz, CDCl₃) δ 7.50 (d, J=8.8 Hz, 2H), 7.39 (d, J=7.6 Hz, 1H), 7.30-7.28 (m, 2H), 7.25-7.21 (m, 1H), 7.09 (d, J=8.8 Hz, 2H), 3.06 (q, J=7.6 Hz, 2H), 1.52 (s, 9H), 1.13 (t, J=7.6 Hz, 3H).

MH+501.

### Example 146

### 2-(1-(4-Bromophenyl)-2-(2-chlorophenyl)-5-ethyl-1H-imidazol-4-yl)-5-(1-(trifluoromethyl)cyclopropyl)-1,3,4-thiadiazole

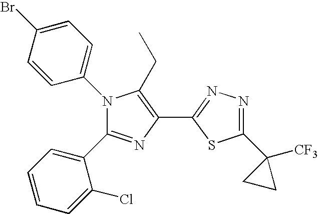

¹H NMR (400 MHz, CDCl₃) δ 7.51 (d, J=8.8 Hz, 2H), 7.38 (d, J=7.2 Hz, 1H), 7.32-7.29 (m, 2H), 7.26-7.22 (m, 1H), 7.09 (d, J=8.8 Hz, 2H), 3.00 (q, J=7.6 Hz, 2H), 1.72-1.69 (m, 2H), 1.62-1.58 (m, 2H), 1.13 (t, J=7.6 Hz, 3H).

MH+553.

### Example 147

### 2-(1-(4-Bromophenyl)-2-(2-chlorophenyl)-5-ethyl-1H-imidazol-4-yl)-5-(1-(trifluoromethyl)cyclobutyl)-1,3,4-thiadiazole

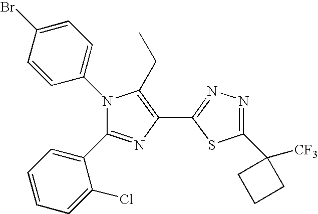

¹H NMR (400 MHz, CDCl₃) δ 7.51 (d, J=8.8 Hz, 2H), 7.38 (d, J=7.2 Hz, 1H), 7.30 (d, J=4.4 Hz, 2H), 7.25-7.22 (m, 1H), 7.10 (d, J=8.8 Hz, 2H), 3.08 (q, J=7.6 Hz, 2H), 2.88-2.79 (m, 4H), 2.20-2.12 (m, 2H), 1.16 (t, J=7.6 Hz, 3H).

MH+567.

### Example 148

### 2-(1-(4-Bromophenyl)-2-(2-chlorophenyl)-5-ethyl-1H-imidazol-4-yl)-5-(1-(4-chlorophenyl)cyclopropyl)-1,3,4-thiadiazole

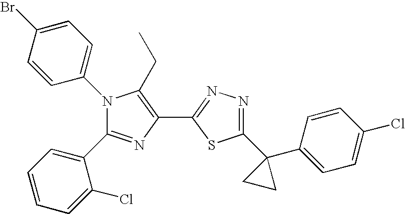

¹H NMR (400 MHz, CDCl₃) δ 7.48 (d, J=8.8 Hz, 2H), 7.43 (d, J=8.8 Hz, 2H), 7.33-7.30 (m, 3H), 7.27-7.26 (m, 2H), 7.22-7.18 (m, 1H), 7.06 (d, J=8.8 Hz, 2H), 3.01 (q, J=7.6 Hz, 2H), 1.90 (dd, J=7.2, 4.4 Hz, 2H), 1.48 (dd, J=7.2, 4.4 Hz, 2H), 1.10 (t, J=7.6 Hz, 3H).

MH+595.

### Example 149

### 2-tert-Butyl-5-(2-(2-chlorophenyl)-1-(4-chlorophenyl)-5-methyl-1H-imidazol-4-yl)-1,3,4-thiadiazole

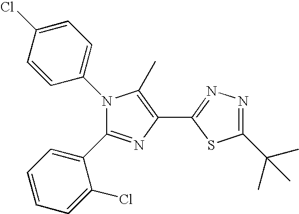

¹H NMR (400 MHz, CDCl₃) δ 7.40 (d, J=7.2 Hz, 1H), 7.34 (d, J=8.0 Hz, 2H), 7.31-7.23 (m, 3H), 7.12 (d, J=8.4 Hz, 2H), 2.60 (s, 3H), 1.52 (s, 9H).

MH+443.

### Example 150

### 2-(2-(2-Chlorophenyl)-1-(4-chlorophenyl)-5-methyl-1H-imidazol-4-yl)-5-(1-(trifluoromethyl)cyclopropyl)-1,3,4-thiadiazole

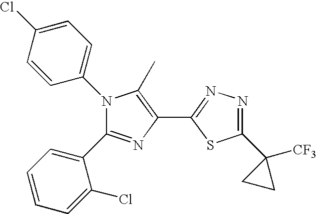

¹H NMR (400 MHz, CDCl₃) δ 7.40 (d, J=7.2 Hz, 1H), 7.34 (d, J=8.8 Hz, 2H), 7.32-7.29 (m, 2H), 7.28-7.24 (m, 1H), 7.11 (d, J=8.8 Hz, 2H), 2.59 (s, 3H), 1.71-1.68 (m, 2H), 1.62-1.58 (m, 2H).

MH+495.

### Example 151

### 2-(2-(2-Chlorophenyl)-1-(4-chlorophenyl)-5-methyl-1H-imidazol-4-yl)-5-(1-(trifluoromethyl)cyclobutyl)-1,3,4-thiadiazole

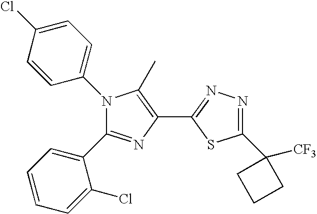

¹H NMR (400 MHz, CDCl₃) δ 7.40 (d, J=6.8 Hz, 1H), 7.35 (d, J=8.8 Hz, 2H), 7.32-7.27 (m, 2H), 7.25-7.23 (m, 1H), 7.12 (d, J=8.8 Hz, 2H), 2.82 (t, J=8.0 Hz, 4H), 2.62 (s, 3H), 2.20-2.12 (m, 2H).

MH+509.

### Example 152

### 2-(2-(2-Chlorophenyl)-1-(4-chlorophenyl)-5-methyl-1H-imidazol-4-yl)-5-(1-(4-chlorophenyl)cyclopropyl)-1,3,4-thiadiazole

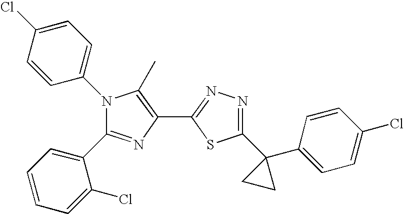

¹H NMR (400 MHz, CDCl₃) δ 7.43 (d, J=8.8 Hz, 2H), 7.34-7.31 (m, 5H), 7.28-7.26 (m, 2H), 7.23-7.19 (m, 1H), 7.09 (d, J=8.8 Hz, 2H), 2.56 (s, 3H), 1.90 (dd, J=6.8, 4.0 Hz, 2H), 1.48 (dd, J=7.2, 4.0 Hz, 2H).

MH+537.

### Example 153

### 2-tert-Butyl-5-(2-(2-chlorophenyl)-1-(4-chlorophenyl)-5-ethyl-1H-imidazol-4-yl)-1,3,4-thiadiazole

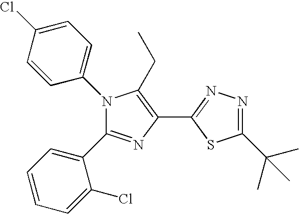

¹H NMR (400 MHz, CDCl₃) δ 7.39 (d, J=8.0 Hz, 1H), 7.34 (d, J=8.8 Hz, 2H), 7.30-7.28 (m, 2H), 7.25-7.21 (m, 1H), 7.16 (d, J=8.8 Hz, 2H), 3.06 (q, J=7.6 Hz, 2H), 1.52 (s, 9H), 1.13 (t, J=7.6 Hz, 3H).

MH+457.

### Example 154

### 2-(2-(2-Chlorophenyl)-1-(4-chlorophenyl)-5-ethyl-1H-imidazol-4-yl)-5-(1-(trifluoromethyl)cyclopropyl)-1,3,4-thiadiazole

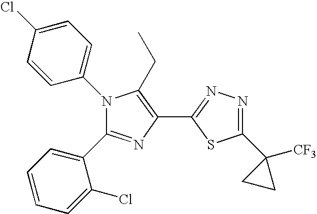

¹H NMR (400 MHz, CDCl₃) δ 7.38 (d, J=7.2 Hz, 1H), 7.35 (d, J=8.8 Hz, 2H), 7.31-7.29 (m, 2H), 7.25-7.22 (m, 1H), 7.16 (d, J=8.8 Hz, 2H), 3.05 (q, J=7.6 Hz, 2H), 1.72-1.69 (m, 2H), 1.62-1.60 (m, 2H), 1.13 (t, J=7.6 Hz, 3H).

MH+509.

### Example 155

### 2-(2-(2-Chlorophenyl)-1-(4-chlorophenyl)-5-ethyl-1H-imidazol-4-yl)-5-(1-(trifluoromethyl)cyclobutyl)-1,3,4-thiadiazole

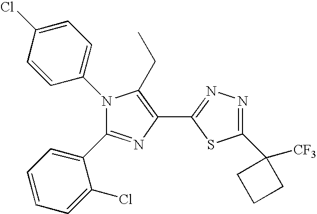

¹H NMR (400 MHz, CDCl₃) δ 7.38 (d, J=7.2 Hz, 1H), 7.35 (d, J=8.8 Hz, 2H), 7.30 (d, J=4.0 Hz, 2H), 7.25-7.22 (m, 1H), 7.16 (d, J=8.8 Hz, 2H), 3.08 (q, J=7.6 Hz, 2H), 2.86-2.79 (m, 4H), 2.20-2.12 (m, 2H), 1.16 (t, J=7.6 Hz, 3H).

MH+523.

### Example 156

### 2-(2-(2-Chlorophenyl)-1-(4-chlorophenyl)-5-ethyl-1H-imidazol-4-yl)-5-(1-(4-chlorophenyl)cyclopropyl)-1,3,4-thiadiazole

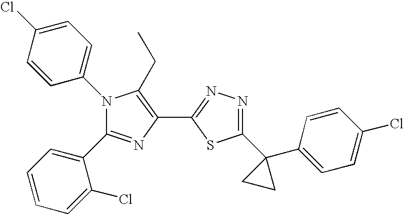

¹H NMR (400 MHz, CDCl₃) δ 7.43 (d, J=8.8 Hz, 2H), 7.34-7.30 (m, 5H), 7.27-7.25 (m, 2H), 7.22-7.18 (m, 1H), 7.13 (d, J=8.8 Hz, 2H), 3.02 (q, J=7.6 Hz, 2H), 1.90 (dd, J=6.8, 4.4 Hz, 2H), 1.48 (dd, J=7.2, 4.4 Hz, 2H), 1.11 (t, J=7.6 Hz,3H).

MH+551.

### Example 157

### 2-(1-(4-Chlorophenyl)-2-(2,4-dichlorophenyl)-5-methyl-1H-imidazol-4-yl)-5-(1-(trifluoromethyl)cyclopropyl)-1,3,4-thiadiazole

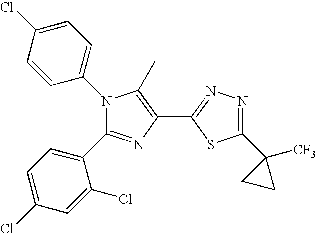

¹H NMR (400 MHz, CDCl₃) δ 7.37 (d, J=8.8 Hz, 2H), 7.34-7.32 (m, 2H), 7.26-7.24 (m, 1H), 7.10 (d, J=8.8 Hz, 2H), 2.59 (s, 3H), 1.70 (s, 2H), 1.62-1.59 (m, 2H).

MH+529.

### Example 158

### 2-(1-(4-Chlorophenyl)-2-(2,4-dichlorophenyl)-5-methyl-1H-imidazol-4-yl)-5-(1-(trifluoromethyl)cyclobutyl)-1,3,4-thiadiazole

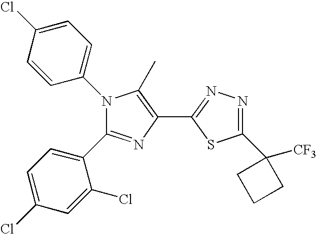

¹H NMR (400 MHz, CDCl₃) δ 7.38 (d, J=8.8 Hz, 2H), 7.34-7.32 (m, 2H), 7.26-7.24 (m, 1H), 7.11 (d, J=8.4 Hz, 2H), 2.82 (t, J=8.0 Hz, 4H), 2.62 (s, 3H), 2.20-2.12 (m, 2H).

MH+543.

### Example 159

### 2-(1-(4-Chlorophenyl)-2-(2,4-dichlorophenyl)-5-methyl-1H-imidazol-4-yl)-5-(1-(4-chlorophenyl)cyclopropyl)-1,3,4-thiadiazole

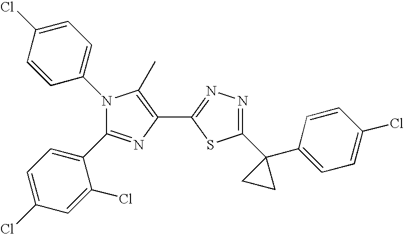

¹H NMR (400 MHz, CDCl₃) δ 7.43 (d, J=8.8 Hz, 2H), 7.36-7.28 (m, 6H), 7.21 (dd, J=8.4, 2.0 Hz, 1H), 7.07 (d, J=8.8 Hz, 2H), 2.55 (s, 3H), 1.90 (dd, J=6.8, 4.4 Hz, 2H), 1.49 (dd, J=6.8, 4.4 Hz, 2H).

MH+571.

### Example 160

### 2-(1-(4-Chlorophenyl)-2-(2,4-dichlorophenyl)-5-ethyl-1H-imidazol-4-yl)-5-(1-(trifluoromethyl)cyclopropyl)-1,3,4-thiadiazole

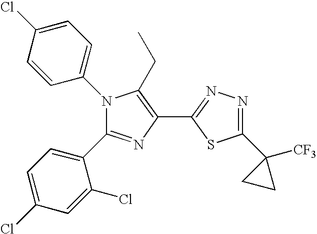

¹H NMR (400 MHz, CDCl₃) δ 7.38 (d, J=8.4 Hz, 2H), 7.33 (d, J=2.0 Hz, 2H), 7.31 (s, 1H), 7.24 (dd, J=8.4, 2.0 Hz, 1H), 7.14 (d, J=8.4 Hz, 2H), 3.04 (q, J=7.6 Hz, 2H), 1.72-1.69 (m, 2H), 1.62-1.59 (m, 2H), 1.13 (t, J=7.6 Hz, 3H).

MH+543.

### Example 161

### 2-(1-(4-Chlorophenyl)-2-(2,4-dichlorophenyl)-5-ethyl-1H-imidazol-4-yl)-5-(1-(trifluoromethyl)cyclobutyl)-1,3,4-thiadiazole

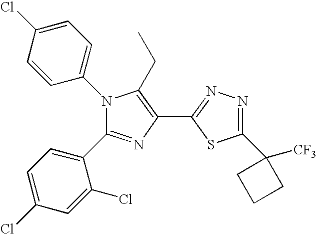

¹H NMR (400 MHz, CDCl₃) δ 7.38 (d, J=8.8 Hz, 2H), 7.33 (d, J=2.0 Hz, 1H), 7.31 (s, 1H), 7.24 (dd, J=8.0, 2.0 Hz, 1H), 7.15 (d, J=8.4 Hz, 2H), 3.07 (q, J=7.6 Hz, 2H), 2.82 (t, J=8.0 Hz, 4H), 2.20-2.12 (m, 2H), 1.15 (t, J=7.6 Hz, 3H).

MH+557.

### Example 162

### 2-(1-(4-Chlorophenyl)-2-(2,4-dichlorophenyl)-5-ethyl-1H-imidazol-4-yl)-5-(1-(4-chlorophenyl)cyclopropyl)-1,3,4-thiadiazole

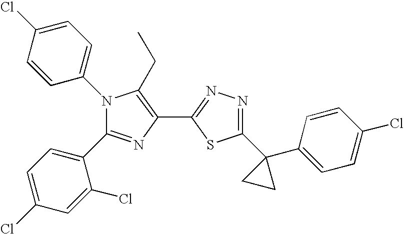

¹H NMR (400 MHz, CDCl₃) δ 7.43 (d, J=8.4 Hz, 2H), 7.37 (s, 1H), 7.34 (d, J=4.4 Hz, 2H), 7.31 (s, 1H), 7.29 (d, J=1.6 Hz, 1H), 7.27-7.25 (m, 1H), 719 (dd, J=8.4, 1.6 Hz, 1H), 7.11 (d, J=8.8 Hz, 2H), 3.01 (q, J=7.2 Hz, 2H), 1.90 (dd, J=6.8, 4.4 Hz, 2H), 1.49 (dd, J=6.8, 4.4 Hz, 2H), 1.10 (t, J=7.2 Hz, 3H).

MH+585.

### Example 163

### 2-(1-(4-Chlorophenyl)-2-(2,4-dichlorophenyl)-5-ethyl-1H-imidazol-4-yl)-5-(trifluoromethyl)-1,3,4-thiadiazole

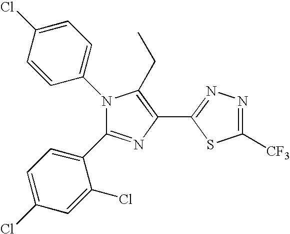

¹H NMR (400 MHz, CDCl₃) δ 7.40 (d, J=8.8 Hz, 2H), 7.35 (d, J=2.0 Hz, 1H), 7.32 (d, J=8.4 Hz, 1H), 7.26-7.24 (m, 1H), 7.15 (d, J=8.4 Hz, 2H), 3.07 (q, J=7.6 Hz, 2H), 1.15 (t, J=7.6 Hz, 3H).

MH+503.

### Example 164

### 2-(5-((1H-1,2,4-Triazol-1-yl)methyl)-1-(4-bromophenyl)-2-(2,4-dichlorophenyl)-1H-imidazol-4-yl)-5-tert-butyl-1,3,4-thiadiazole

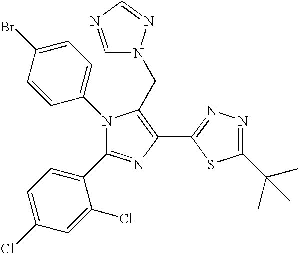

¹H NMR (400 MHz, CDCl₃) δ 8.49 (s, 1H), 7.84 (s, 1H), 7.53 (d, J=8.4 Hz, 2H), 7.36-7.23 (m, 3H), 7.17 (d, J=8.4 Hz, 2H), 5.74 (s, 2H), 1.54 (s, 9H).

MH+588.

### Example 165

### 2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclopropyl)-1,3,4-thiadiazole

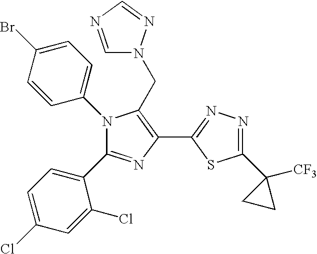

¹H NMR (400 MHz, CDCl₃) δ 8.42 (s, 1H), 7.84 (s, 1H), 7.54 (d, J=8.4 Hz, 2H), 7.36 (d, J=2.0 Hz, 1H), 7.31 (d, J=8.8Hz, 1H), 7.26-7.24 (m, 1H), 7.18 (d, J=8.4 Hz, 2H), 5.72 (s, 2H), 1.72-1.70 (m, 2H), 1.66-1.61 (m, 2H).

MH+640.

### Example 166

### 2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclobutyl)-1,3,4-thiadiazole

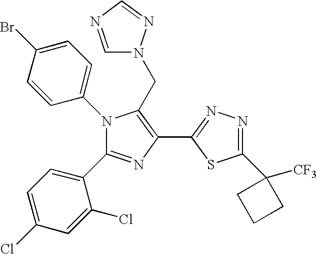

¹H NMR (400 MHz, CDCl₃) δ 8.48 (s, 1H), 7.85 (s, 1H), 7.54 (d, J=8.8 Hz, 2H), 7.36 (d, J=2.4 Hz, 1H), 7.30 (d, J=8.4 Hz, 1H), 7.26-7.24 (m, 1H), 7.19 (d, J=8.8 Hz, 2H), 5.75 (s, 2H), 2.89-2.80 (m, 4H), 2.22-2.14 (m, 2H).

MH+654.

### Example 167

### 2-(5-((1H-1,2,4-Triazol-1-yl)methyl)-1-(4-bromophenyl)-2-(2,4-dichlorophenyl)-1H-imidazol-4-yl)-5-(1-(4-chlorophenyl)cyclopropyl)-1,3,4-thiadiazole

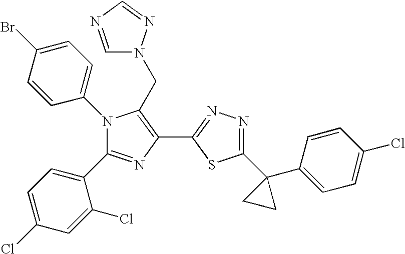

¹H NMR (400 MHz, CDCl₃) δ 8.43 (s, 1H), 7.83 (s, 1H), 7.52 (d, J=8.8 Hz, 2H), 7.43 (d, J=8.8 Hz, 2H), 7.35 (s, 1H), 7.33-7.32 (m, 2H), 7.24 (s, 1H), 7.21 (dd, J=8.4, 2.0 Hz, 1H), 7.15 (d, J=8.8 Hz, 2H), 5.69 (s, 2H), 1.92 (dd, J=7.2, 4.4 Hz, 2H), 1.53 (dd, J=7.2, 4.4 Hz, 2H).

MH+682.

### Example 168

### 2-(5-((1H-1,2,4-Triazol-1-yl)methyl)-1-(4-bromophenyl)-2-(2-chlorophenyl)-1H-imidazol-4-yl)-5-tert-butyl-1,3,4-thiadiazole

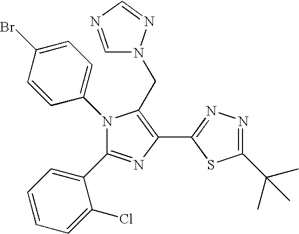

¹H NMR (400 MHz, CDCl₃) δ 8.49 (s, 1H), 7.84 (s, 1H), 7.50 (d, J=8.8 Hz, 2H), 7.36 (d, J=7.2 Hz, 1H), 7.32 (d, J=3.6 Hz, 2H), 7.27-7.23 (m, 1H), 7.17 (d, J=8.8 Hz, 2H), 5.75 (s, 2H), 1.54 (s, 9H).

MH+554.

### Example 169

### 2-(5-((1H-1,2,4-Triazol-1-yl)methyl)-1-(4-bromophenyl)-2-(2-chlorophenyl)-1H-imidazol-4-yl)-5-(1-(trifluoromethyl)cyclopropyl)-1,3,4-thiadiazole

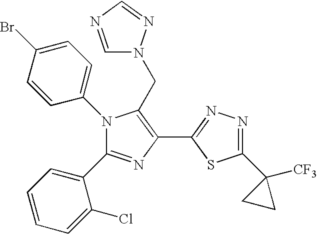

¹H NMR (400 MHz, CDCl₃) δ 8.41 (s, 1H), 7.84 (s, 1H), 7.50 (d, J=8.8 Hz, 2H), 7.37 (d, J=7.2 Hz, 1H), 7.34-7.33 (m, 2H), 7.28-7.24 (m, 1H), 7.18 (d, J=8.8 Hz, 2H), 5.73 (s, 2H), 1.73-1.61 (m, 4H).

MH+606.

### Example 170

### 2-(5-((1H-1,2,4-Triazol-1-yl)methyl)-1-(4-bromophenyl)-2-(2-chlorophenyl)-1H-imidazol-4-yl)-5-(1-(trifluoromethyl)cyclobutyl)-1,3,4-thiadiazole

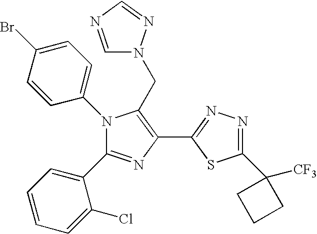

¹H NMR (400 MHz, CDCl₃) δ 8.47 (s, 1H), 7.85 (s, 1H), 7.51 (d, J=8.8 Hz, 2H), 7.36 (d, J=7.2 Hz, 1H), 7.34-7.32 (m, 2H), 7.29-7.24 (m, 1H), 7.19 (d, J=8.8 Hz, 2H), 5.76 (s, 2H), 2.86-2.80 (m, 4H), 2.22-2.16 (m, 2H).

MH+620.

### Example 171

### 2-(5-((1H-1,2,4-Triazol-1-yl)methyl)-1-(4-bromophenyl)-2-(2-chlorophenyl)-1H-imidazol-4-yl)-5-(1-(4-chlorophenyl)cyclopropyl)-1,3,4-thiadiazole

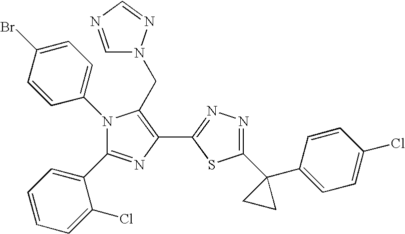

¹H NMR (400 MHz, CDCl₃) δ 8.42 (s, 1H), 7.83 (s, 1H), 7.48 (d, J=8.8 Hz, 2H), 7.43 (d, J=8.4 Hz, 2H), 7.35-7.28 (m, 5H), 7.23-7.19 (m, 1H), 7.14 (d, J=8.8 Hz, 2H), 5.70 (s, 2H), 1.91 (dd, J=6.8, 4.2 Hz, 2H), 1.53 (dd, J=6.8, 4.2 Hz, 2H).

MH+648.

### Example 172

### 2-(5-((1H-1,2,4-Triazol-1-yl)methyl)-2-(2-chlorophenyl)-1-(4-chlorophenyl)-1H-imidazol-4-yl)-5-tert-butyl-1,3,4-thiadiazole

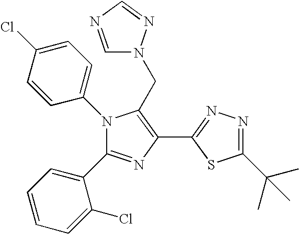

¹H NMR (400 MHz, CDCl₃) δ 8.49 (s, 1H), 7.84 (s, 1H), 7.38-7.32 (m, 6H), 7.27-7.22 (m, 2H), 5.75 (s, 2H), 1.54 (s, 9H).

MH+510.

### Example 173

### 2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclopropyl)-1,3,4-thiadiazole

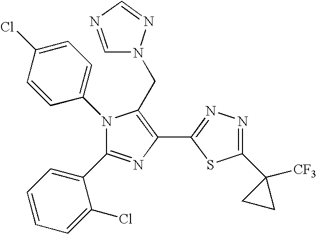

¹H NMR (400 MHz, CDCl₃) δ 8.41 (s, 1H), 7.84 (s, 1H), 7.42-7.33 (m, 6H), 7.28-7.23 (m, 2H), 5.67 (s, 2H), 1.73-1.69 (m, 2H), 1.66-1.63 (m, 2H).

MH+562.

### Example 174

### 2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclobutyl)-1,3,4-thiadiazole

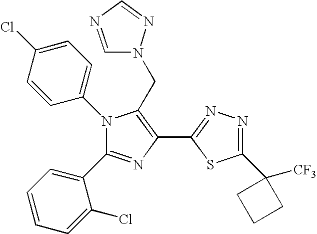

¹H NMR (400 MHz, CDCl₃) δ 8.47 (s, 1H), 7.85 (s, 1H), 7.38-7.31 (m, 6H), 7.29-7.24 (m, 2H), 5.77 (s, 2H), 2.86-2.82 (m, 4H), 2.22-2.14 (m, 2H).

MH+576.

### Example 175

### 2-(5-((1H-1,2,4-Triazol-1-yl)methyl)-2-(2-chlorophenyl)-1-(4-chlorophenyl)-1H-imidazol-4-yl)-5-(1-(4-chlorophenyl)cyclopropyl)-1,3,4-thiadiazole

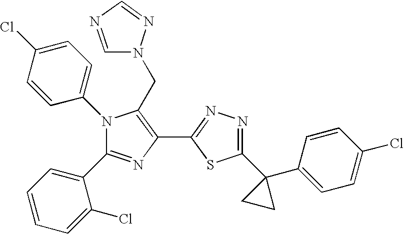

¹H NMR (400 MHz, CDCl₃) δ 8.42 (s, 1H), 7.83 (s, 1H), 7.43 (d, J=8.4 Hz, 2H), 7.35-7.29 (m, 7H), 7.23-7.20 (m, 3H), 5.71 (s, 2H), 1.91 (dd, J=7.2, 4.4 Hz, 2H), 1.53 (dd, J=7.2, 4.4 Hz, 2H).

MH+604.

### Example 176

### 2-(5-((1H-1,2,4-Triazol-1-yl)methyl)-1-(4-chlorophenyl)-2-(2,4-dichlorophenyl)-1H-imidazol-4-yl)-5-tert-butyl-1,3,4-thiadiazole

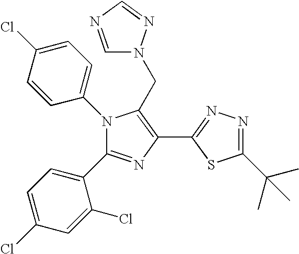

¹H NMR (400 MHz, CDCl₃) δ 9.50 (s, 1H), 8.24 (s, 1H), 7.42 (d, J=8.4 Hz, 2H), 7.37-7.34 (m, 2H), 7.28-7.26 (m, 3H), 5.83 (s, 2H), 1.54 (s, 9H).

MH+544.

### Example 177

### 2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclopropyl)-1,3,4-thiadiazole

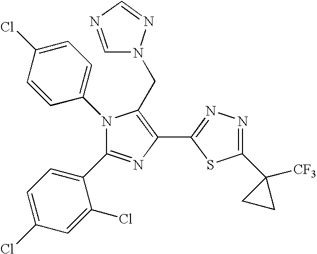

¹H NMR (400 MHz, CDCl₃) δ 9.36 (s, 1H), 8.27 (s, 1H), 7.46 (d, J=2.0 Hz, 2H), 7.42-7.36 (m, 4H), 7.33 (dd, J=8.4, 2.0 Hz, 1H), 5.69 (s, 2H), 1.76 (s, 2H), 1.67-1.65 (m, 2H).

MH+596.

### Example 178

### 2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclobutyl)-1,3,4-thiadiazole

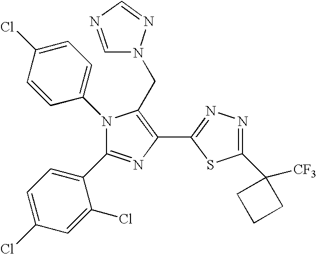

¹H NMR (400 MHz, CDCl₃) δ 8.48 (s, 1H), 7.85 (s, 1H), 7.40-7.36 (m, 3H), s 7.30 (d, J=8.0 Hz, 2H), 7.26-7.23 (m, 2H), 5.75 (s, 2H), 2.89-2.78 (m, 4H), 2.22-2.14 (m, 2H).

MH+610.

### Example 179

### 2-(5-((1H-1,2,4-Triazol-1-yl)methyl)-1-(4-chlorophenyl)-2-(2,4-dichlorophenyl)-1H-imidazol-4-yl)-5-(1-(4-chlorophenyl)cyclopropyl)-1,3,4-thiadiazole

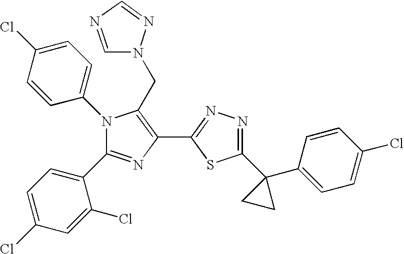

¹H NMR (400 MHz, CDCl₃) δ 9.33 (s, 1H), 8.18 (s, 1H), 7.44-7.38 (m, 4H), 7.35-7.32 (m, 3H), 7.29-7.22 (m, 4H), 5.77 (s, 2H), 1.92 (dd, J=7.2, 4.4 Hz, 2H), 1.55 (dd, J=7.2, 4.4 Hz, 2H).

MH+638.

### Preparation of Oxazole Formula (1c))

### Example 180

### 2-(1-(4-Chlorophenyl)-2-(2,4-dichlorophenyl)-5-methyl-1H-imidazol-4-yl)-5-cyclohexyloxazole

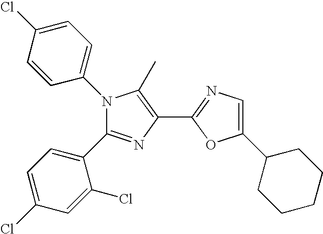

### Step 1: tert-Butyl 2-(methoxy(methyl)amino)-2-oxoethylcarbamate

To a 2-(tert-butoxycarbonylamino)acetic acid (800 mg, 4.57 mmol), N,O-dimethylhydroxylamine hydrochloride (535 mg, 5.48 mmol), EDCI (1.05 g, 5.48 mmol), HOBt (740 mg, 5.48 mmol) in DCM (25 mL) was added NMM (2.77 g, 27.42 mmol) at rt. The reaction mixture was stirred overnight. After the reaction was completed, DCM was evaporated in vacuo. The residue was dissolved in MeOH, filtered through a syringe filter, and then purified by reverse-phase prep HPLC to provide the desired product (705 mg, 71%) as a white solid

¹H NMR (400 MHz, CDCl₃) δ 5.27 (br, 1H), 4.09 (d, J=4.6 Hz, 2H), 3.72 (s, 3H), 3.21 (s, 3H) 1.46 (s, 9H).

MH+219.

### Step 2: tert-Butyl 2-cyclohexyl-2-oxoethylcarbamate

To a tert-butyl 2-(methoxy(methyl)amino)-2-oxoethylcarbamate (400 mg, 1.83 mmol) in THF (10 mL) was added cyclohexylmagnesium chloride (4.12 mL, 8.24 mmol) portionwise at rt under N₂ atmosphere. As addition of the Grignard reagent was completed, the reaction mixture was stirred and heated to reflux for an hour. Then the mixture was allowed to cool down to rt. The reaction was quenched by adding water (10 mL) slowly. 1N HCl solution (30 mL) was added to the mixture, and extracted with EtOAc (20 mL×3). The combined organic layers were collected and evaporated in vacuo. The crude material was purified by column chromatography (hexane: EtOAc=10:1 to 5: 1) to yield the desired product (110 mg, 25%).

¹H NMR (400 MHz, CDCl₃) δ 5.26 (br, 1H), 4.07 (d, J=4.2 Hz, 2H), 2.41-2.34 (m, 1H), 1.86-1.67 (m, 5H), 1.45 (s, 9H), 1.43-1.19 (m, 5H).

MH+242.

### Step3: 1-(4-Chlorophenyl)-N-(2-cyclohexyl-2-oxoethyl)-2-(2,4-dichlorophenyl)-5-methyl-1H-imidazole-4-carboxamide

To a tert-butyl 2-cyclohexyl-2-oxoethylcarbamate (85 mg, 0.423 mmol) in DCM (2 mL) was added TFA (1 mL) at rt. The reaction continued for an hour at rt. The volatiles were removed under vacuum. To the residue (2-amino-1-cyclohexylethanone trifluoroacetic acid), 1-(4-chlorophenyl)-2-(2,4-dichlorophenyl)-5-methyl-1H-imidazole-4-carboxylic acid (161 mg, 0.423 mmol), EDCI (81.1 mg, 0.423 mmol), HOBt (57.2 mmol, 0.423 mmol) in DCM (3 mL) was added NMM (0.43 g, 4.23 mmol)at rt. The reaction mixture was stirred overnight. DCM was removed by evaporation under vacuum. The residue was purified by reverse-phase prep HPLC to afford the title compound (29 mg, 16%).

¹H NMR (300 MHz, CDCl₃) δ 7.76 (br, 1H), 7.39-7.31 (m, 3H), 7.26-7.21 (m, 2H), 7.03 (d, J=8.6 Hz, 2H), 4.34 (d, J=5.1 Hz), 2.52-2.42 (m, 4H), 1.91-1.67 (m, 5H), 1.49-1.16 (m, 5H).

MH+504.

### Step 4: 2-(1-(4-chlorophenyl)-2-(2,4-dichlorophenyl)-5-methyl-1H-imidazol-4-yl)-5-cyclohexyloxazole

1-(4-Chlorophenyl)-N-(2-cyclohexyl-2-oxoethyl)-2-(2,4-dichlorophenyl)-5-methyl-1H-imidazole-4-carboxamide (29 mg, 0.0574 mmol) obtained in Step 3 was added to a microwave reactor containing Burgess reagent (27.4 mg, 0.115 mmol) in THF (1 mL). The capped reactor was 10 placed in a microwave reactor and the mixture was irradiated at 160° C. for 30 min. The reaction product was purified by reverse-phase prep HPLC to provide the title compound (6.2 mg, 22%) as a white solid.

¹H NMR (400 MHz, CDCl₃) δ 7.39-7.30 (m, 4H), 7.25-7.22 (m, 1H), 7.09-7.05 (m, 2H), 6.80 (s, 1H), 2.79-2.75 (m, 1H), 2.49 (s, 3H), 2.11 (d, J=12.4 Hz, 2H), 1.82-1.79 (m, 2H), 1.70-1.65 (m, 2H), 1.49-1.25 (m, 4H).

MH+486

The following compounds of Examples 181 to 192 were obtained by repeating the procedure of Example 180.

### Example 181

### 2-(1-(4-Chlorophenyl)-2-(2,4-dichlorophenyl)-5-methyl-1H-imidazol-4-yl)-5-ethyloxazole

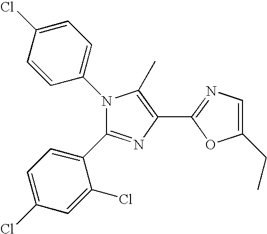

¹H NMR (300 MHz, CDCl₃) δ 7.40-7.35 (m, 3H), 7.31 (d, J=2.0 Hz, 1H), 7.24 (dd, J=8.2, 2.2 Hz, 1H), 7.10-7.07 (m, 2H), 6.84 (s, 1H), 2.77 (q, J=7.5 Hz, 2H), 2.51 (s, 3H), 1.30 (t, J=7.5 Hz, 3H).

MH+432.

### Example 182

### 5-Butyl-2-(1-(4-chlorophenyl)-2-(2,4-dichlorophenyl)-5-methyl-1H-imidazol-4-yl)oxazole

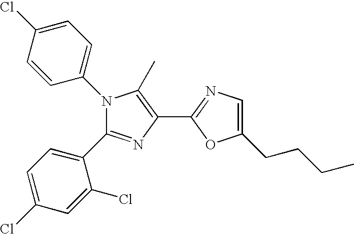

¹H NMR (300 MHz, CDCl₃) δ 7.40-7.22 (m, 5H), 7.10-7.07 (m, 2H), 6.84 (s, 1H), 2.73 (t, J=7.5 Hz, 2H), 2.51 (s, 3H), 1.72-1.64 (m, 2H), 1.44-1.37 (m, 2H), 0.94 (t, J=7.3 Hz, 3H).

MH+460.

### Example 183

### 2-(1-(4-Chlorophenyl)-2-(2,4-dichlorophenyl)-5-methyl-1H-imidazol-4-yl)-5-isopropyloxazole

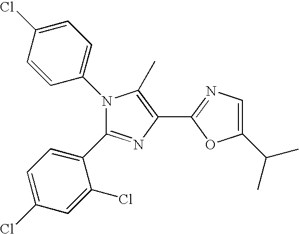

¹H NMR (300 MHz, CDCl₃) δ 7.41-7.34 (m, 3H), 7.30 (d, J=2.0 Hz, 1H), 7.24 (dd, J=8.2, 2.0 Hz, 1H), 7.09-7.07 (m, 2H), 6.81 (s, 1H), 3.11-3.06 (m, 1H), 2.50 (s, 3H), 1.32 (d, J=7.0 Hz, 6H).

MH+446.

### Example 184

### 2-(1-(4-Chlorophenyl)-2-(2,4-dichlorophenyl)-5-methyl-1H-imidazol-4-yl)-5-isobutyloxazole

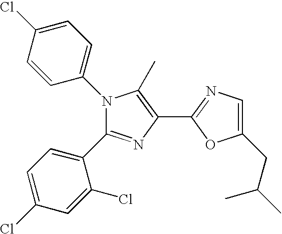

¹H NMR (400 MHz, CDCl₃) δ 7.40-7.22 (m, 5H), 7.09-7.07 (m, 2H), 6.85 (s, 1H), 2.60 (d, J=6.8 Hz, 2H), 2.51 (s, 3H), 2.08-2.03 (m, 1H), 0.97 (d, J=6.6 Hz, 6H).

MH+460.

### Example 185

### 2-tert-Butyl-5-(1-(4-chlorophenyl)-2-(2,4-dichlorophenyl)-5-methyl-1H-imidazol-4-yl)oxazole

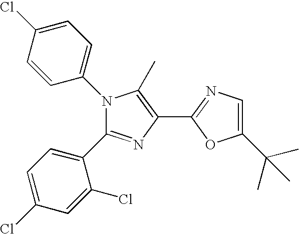

¹H NMR (400 MHz, CDCl₃) δ 7.41-7.33 (m, 3H), 7.30 (d, J=1.8 Hz, 1H), 7.24 (dd, J=8.2, 1.8 Hz, 1H), 7.09-7.05 (m, 2H), 6.79 (s, 1H), 2.49 (s, 3H), 1.36 (s, 9H).

MH+460.

### Example 186

### 2-tert-Butyl-5-(1-(4-chlorophenyl)-2-(2,4-dichlorophenyl)-5-ethyl-1H-imidazol-4-yl)oxazole

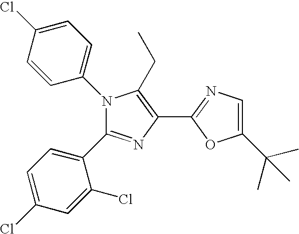

¹H NMR (400 MHz, CDCl₃) δ 7.39-7.34 (m, 3H), 7.29 (d, J=2.3 Hz, 1H), 7.22 (dd, J=8.2, 2.3 Hz, 1H), 7.13-7.10 (m, 2H), 6.80 (s, 1H), 2.95 (q, J=7.3 Hz, 2H), 1.36 (s, 9H), 1.09 (t, J=7.3 Hz, 3H).

MH+474.

### Example 187

### 2-(1-(4-Bromophenyl)-2-(2,4-dichlorophenyl)-5-methyl-1H-imidazol-4-yl)-5-tert-butyloxazole

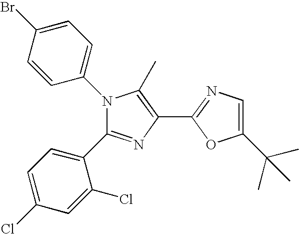

¹H NMR (400 MHz, CDCl₃) δ 7.53-7.50 (m, 2H), 7.42-7.40 (m, 1H), 7.31-7.30 (m, 1H), 7.27-7.23 (m, 1H), 7.02-7.00 (m, 2H), 6.80 (s, 1H), 2.49 (s, 3H), 1.37 (s, 9H).

MH+504.

### Example 188

### 2-(1-(4-Bromophenyl)-2-(2,4-dichlorophenyl)-5-ethyl-1H-imidazol-4-yl)-5-tert-butyloxazole

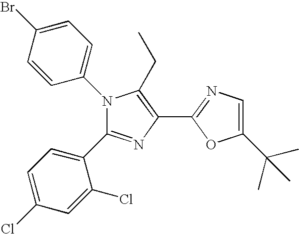

¹H NMR (400 MHz, CDCl₃) δ 7.52-7.50 (m, 2H), 7.38 (d, J=8.4 Hz, 1H), 7.30 (d, J=2.0 Hz, 1H), 7.22 (dd, J=8.4, 2.0 Hz, 1H), 7.06-7.04 (m, 2H), 6.80 (s, 1H), 2.95 (q, J=7.2 Hz, 2H), 1.36 (s, 9H), 1.09 (t, J=7.6 Hz, 3H).

MH+518.

### Example 189

### 4-tert-Butyl-2-(1-(4-chlorophenyl)-2-(2,4-dichlorophenyl)-5-methyl-1H-imidazol-4-yl)oxazole

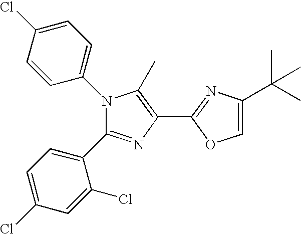

¹H NMR (400 MHz, CDCl₃) δ 7.39-7.34 (m, 4H), 7.29 (d, J=2.0 Hz, 1H), 7.23 (dd, J=8.4, 2.0 Hz, 1H), 7.07 (d, J=8.4 Hz, 2H), 2.51 (s, 3H), 1.32 (s, 9H).

MH+460.

### Example 190

### 4-tert-Butyl-2-(1-(4-chlorophenyl)-2-(2,4-dichlorophenyl)-5-ethyl-1H-imidazol-4-yl)oxazole

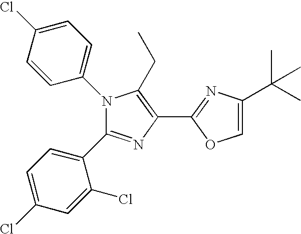

¹H NMR (400 MHz, CDCl₃) δ 7.37-7.34 (m, 4H), 7.29 (d, J=2.0 Hz, 1H), 7.21 (dd, J=8.4, 2.0 Hz, 1H), 7.11 (d, J=8.4 Hz, 2H), 2.96 (q, J=7.6 Hz, 2H), 1.32 (s, 9H), 1.09 (t, J=7.6 Hz, 3H).

MH+474.

### Example 191

### 2-(1-(4-Bromophenyl)-2-(2,4-dichlorophenyl)-5-ethyl-1H-imidazol-4-yl)-4-tert-butyloxazole

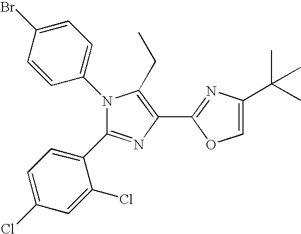

¹H NMR (400 MHz, CDCl₃) δ 7.51 (d, J=8.8 Hz, 2H), 7.36-7.33 (m, 2H), 7.29 (d, J=2.0 Hz, 1H), 7.21 (dd, J=8.4, 2.0 Hz, 2H), 7.04 (d, J=8.8 Hz, 2H), 2.96 (q, J=7.6 Hz, 2H), 1.32 (s, 9H), 1.09 (t, J=7.6 Hz, 3H).

MH+518.

### Example 192

### 2-(1-(4-Chlorophenyl)-2-(2,4-dichlorophenyl)-5-methyl-1H-imidazol-4-yl)-5-ethyl-4-methyloxazole

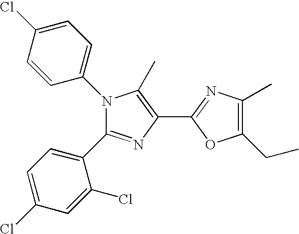

¹H NMR (400 MHz, CDCl₃) ) δ 7.39 (d, J=8.4 Hz, 1H), 7.35 (d, J=8.8 Hz, 2H), 7.29 (d, J=2.0 Hz, 1H), 7.23 (dd, J=8.4, 2.4 Hz, 1H), 7.07 (d, J=8.8 Hz, 1H), 2.69 (q, J=7.6 Hz, 2H), 2.49 (s, 3H), 2.17 (s, 3H), 1.26 (t, J=7.6 Hz, 3H).

MH+446.

### Preparation of Thiazole Formula (Id))

### Example 193

### 2-(1-(4-Chlorophenyl)-2-(2,4-dichlorophenyl)-5-methyl-1H-imidazol-4-yl)-5-cyclohexylthiazole

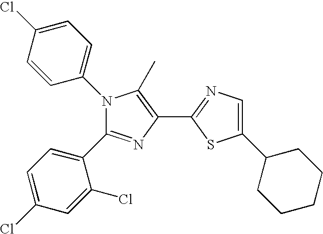

1-(4-Chlorophenyl)-N-(2-cyclohexyl-2-oxoethyl)-2-(2,4-dichlorophenyl)-5-methyl-1H-imidazole-4-carboxamide (35 mg, 0.0693 mmol) was added to a microwave reactor containing Lawesson's reagent (0.18 g, 0.44 mmol) in THF (1 mL). The capped reactor was placed in a microwave reactor and the mixture was irradiated at 160° C. for 30 min. The reaction product was purified by reverse-phase prep HPLC to provide the title compound (11.3 mg, 32%) as a white solid.

¹H NMR (300 MHz, CDCl₃) δ 7.50 (s, 1H), 7.38-7.33 (m, 4H), 7.23 (dd, J=8.2, 2.0 Hz, 1H), 7.10-7.06 (m, 2H), 2.89-2.83 (m, 1H), 2.53 (s, 3H), 2.09-2.05 (m, 2H), 1.85-1.71 (m, 2H), 1.53-1.25 (m, 6H).

MH+502.

The following compounds of Examples 194 to 202 were obtained by repeating the procedure of Example 193.

### Example 194

### 2-(1-(4-Chlorophenyl)-2-(2,4-dichlorophenyl)-5-methyl-1H-imidazol-4-yl)-5-ethylthiazole

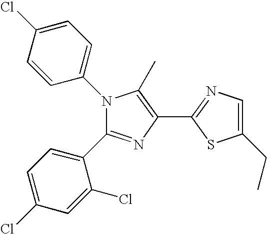

¹H NMR (300 MHz, CDCl₃) δ 7.49 (s, 1H) 7.38-7.34 (m, 3H), 7.31 (d, J=1.8 Hz, 1H), 7.23 (dd, J=8.2, 2.0 Hz, 1H),7.10-7.02 (m, 2H), 2.89 (q, J=7.5 Hz, 2H), 2.53 (s, 3H), 1.34 (t, J=7.5 Hz, 3H).

MH+448.

### Example 195

### 5-Butyl-2-(1-(4-chlorophenyl)-2-(2,4-dichlorophenyl)-5-methyl-1H-imidazol-4-yl)thiazole

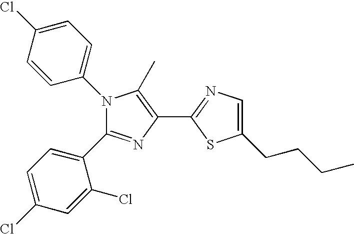

¹H NMR (300 MHz, CDCl₃) δ 7.48 (s, 1H) 7.38-7.33 (m, 3H), 7.31 (d, J=1.8 Hz, 1H), 7.24 (dd, J=8.2, 2.0 Hz, 1H), 7.10-7.07 (m, 2H), 2.85 (t, J=7.0 Hz, 2H), 2.53 (s, 3H), 1.73-1.62 (m, 2H), 1.47-1.34 (m, 2H), 0.93 (t, J=7.3 Hz, 3H).

MH+476.

### Example 196

### 2-(1-(4-Chlorophenyl)-2-(2,4-dichlorophenyl)-5-methyl-1H-imidazol-4-yl)-5-isopropylthiazole

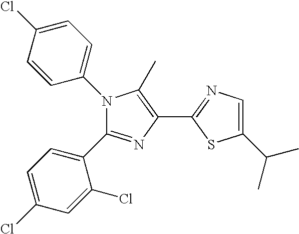

¹H NMR (300 MHz, CDCl₃) δ 7.52 (s, 1H), 7.39-7.34 (m, 3H), 7.31 (d, J=2.0 Hz, 1H), 7.23 (dd, J=8.2, 2.0 Hz, 1H), 7.10-7.07 (m, 2H), 3.27-3.18 (m, 1H), 2.52 (s, 3H), 1.36 (d, J=6.8 Hz, 6H).

MH+462.

### Example 197

### 2-(1-(4-Chlorophenyl)-2-(2,4-dichlorophenyl)-5-methyl-1H-imidazol-4-yl)-5-isobutylthiazole

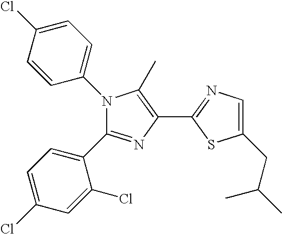

¹H NMR (400 MHz, CDCl₃) δ 7.47 (s, 1H), 7.38-7.22 (m, 5H), 7.08 (d, J=8.0 Hz, 2H) 2.71 (d, J=7.2 Hz, 2H), 2.53 (s, 3H), 1.93-1.86 (m, 1H), 0.97 (d, J=6.4 Hz, 6H).

MH+476.

### Example 198

### 2-tert-Butyl-5-(1-(4-chlorophenyl)-2-(2,4-dichlorophenyl)-5-methyl-1H-imidazol-4-yl)thiazole

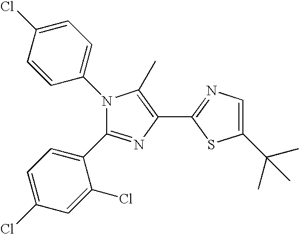

¹H NMR (400 MHz, CDCl₃) δ 7.52 (s, 1H), 7.37-7.34 (m, 3H), 7.31 (d, J=1.8 Hz, 1H), 7.23 (dd, J=8.2, 1.8 Hz, 1H), 7.08 (d, J=8.2 Hz, 2H), 2.52 (s, 3H), 1.42 (s, 9H).

MH+476.

### Example 199

### 2-tert-Butyl-5-(1-(4-chlorophenyl)-2-(2,4-dichlorophenyl)-5-ethyl-1H-imidazol-4-yl)thiazole

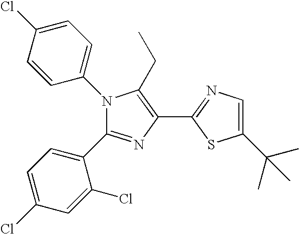

¹H NMR (400 MHz, CDCl₃) δ 7.51 (s, 1H), 7.37-7.33 (m, 3H), 7.30 (d, J=2.3 Hz, 1H), 7.21 (dd, J=8.2, 1.8 Hz, 1H), 7.14-7.11 (m, 2H), 3.01 (q, J=7.3 Hz, 2H), 1.42 (s, 9H), 1.07 (t, J=7.3 Hz, 3H).

MH+490.

### Example 200

### 2-(1-(4-Bromophenyl)-2-(2,4-dichlorophenyl)-5-methyl-1H-imidazol-4-yl)-5-tert-butylthiazole

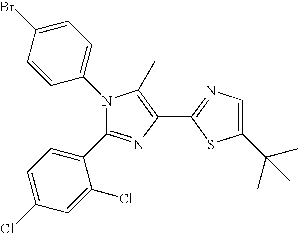

¹H NMR (400MHz, CDCl₃) δ 7.53-7.49 (m, 3H), 7.38-7.36 (m, 1H), 7.31 (d, J=1.8 Hz, 1H), 7.23 (dd, J=8.2, 1.8 Hz, 1H), 7.04-7.00 (m, 2H), 2.53 (s, 3H), 1.42 (s, 9H).

MH+520.

### Example 201

### 2-(1-(4-Bromophenyl)-2-(2,4-dichlorophenyl)-5-ethyl-1H-imidazol-4-yl)-5-tert-butylthiazole

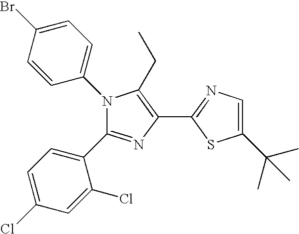

¹H NMR (400 MHz, CDCl₃) δ 7.52-7.50 (m, 3H), 7.36-7.34 (m, 2H), 7.31 (d, J=1.6 Hz, 1H), 7.22 (dd, J=8.4, 2.0 Hz, 1H), 7.08-7.05 (m, 2H), 3.02 (q, J=7.6 Hz, 2H), 1.42 (s, 9H), 1.07 (t, J=7.6 Hz, 3H).

MH+534.

### Example 202

### 2-(1-(4-Chlorophenyl)-2-(2,4-dichlorophenyl)-5-methyl-1H-imidazol-4-yl)-5-ethyl-4-methylthiazole

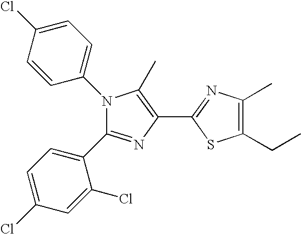

¹H NMR (400 MHz, CDCl₃) δ 7.38-7.33 (m, 3H), 7.30 (d, J=2.0 Hz, 1H), 7.23 (dd, J=8.4, 2.4 Hz, 1H), 7.07 (d, J=8.4 Hz, 2H), 2.78 (q, J=7.6 Hz, 2H), 2.52 (s, 3H), 2.38 (s, 3H), 1.28 (t, J=7.6 Hz, 3H).

MH+462.

### Pharmacological Test

### In vitro Activity Analysis

The compounds of the present invention were analyzed for their binding characteristics for CB₁ and CB₂ and the pharmacological activity thereof in accordance with the method disclosed in [Devane W A, Dysarz F A 3^{rd}, Johnson M R, Melvin L S and Howlett A C, Determination and characterization of a cannabinoid receptor in rat brain, Mol. Pharmacol., 34(5): 605-13(1998)]. The analysis was performed using [³H]CP-55940 which is a selectively radioactivity-labeled 5-(1,1-dimethyheptyl)-2[5-hydroxy-2-(3-hydroxypropyl)-cyclohexyl]-phenol, purchased from PerkinElmer Life Sciences, Inc. (Boston, Mass., U.S.A.), through a rat CB-1 receptor binding protocol as follows.

The tissue obtained from the brain of SD rats was homogenized with a Dounce homogenate system in TME(50 mM Tris, 3 mM MgCl₂ and 1 mM EDTA, pH 7.4) at 4° C., and the homogenate was centrifuged at 48,000 g for 30 min. at 4 ° C. The pellet was resuspended in 5 ml of TME and the suspension was divided into aliquots and stored at −70 ° C. until its use in the following assay.

2 μl of the test compound was diluted in dimethylsulphoxide and was added to a deep well of a polypropylene plate, to which 50 μl of [³H]CP-55940 diluted in a ligand buffer solution (0.1% bovine serum albumin(BAS)+TME) was added. The tissue concentrations were determined by Bradford protein analysis, and 148 μl of brain tissue of the required concentration was added to the plate. The plate was covered and placed in a 30 ° C. incubator for 60 min, and then transformed on GF/B filtermat pretreated in polyethylenimine (PEI) using a cell harvester. Each filter was washed five times and dried at 60 ° C. for 1 hr. Then, the degree of radioactivity retained by the filter was measured using Wallac Microbeta™ (PerkinElmer Life Sciences, Inc., Mass., U.S.A.) and the activity of the compound for inhibiting CB₁ receptor was determined there from and compared with that of the control, rimonabant which is known as a cannabinoid CB₁ receptor antagonist. The results are shown in Table 1.

TABLE 1

| Example | IC₅₀ (nM) |
|---|---|
| 1 | 13.2 |
| 2 | 115 |
| 3 | 35.3 |
| 4 | 38.7 |
| 5 | 27.6 |
| 6 | 12.9 |
| 7 | 14.2 |
| 8 | 39.4 |
| 9 | 25.8 |
| 10 | 22.3 |
| 11 | 31.4 |
| 12 | 48.4 |
| 13 | 25.8 |
| 14 | 34.1 |
| 15 | 18.1 |
| 16 | 11.2 |
| 17 | 11.5 |
| 18 | 17.9 |
| 19 | 70.0 |
| 20 | 5.81 |
| 21 | 3.15 |
| 22 | 29.3 |
| 23 | 24.1 |
| 24 | 79.9 |
| 25 | 21.2 |
| 26 | 10.3 |
| 27 | 9.14 |
| 28 | 15.5 |
| 29 | 71.5 |
| 30 | 392 |
| 31 | 46.1 |
| 32 | 68.9 |
| 33 | 19.2 |
| 34 | 9.05 |
| 35 | 18.9 |
| 36 | 60.1 |
| 37 | 15.1 |
| 38 | 11.7 |
| 39 | 86.9 |
| 40 | 9.05 |
| 41 | 13.6 |
| 42 | 18.6 |
| 43 | 8.38 |
| 44 | 8.05 |
| 45 | 11.4 |
| 46 | 15.6 |
| 47 | 27.6 |
| 48 | 18.7 |
| 49 | 16.7 |
| 50 | 9.56 |
| 51 | 31.7 |
| 52 | 18.6 |
| 53 | 11.4 |
| 55 | 14.7 |
| 56 | 20.1 |
| 57 | 13.4 |
| 58 | 10.3 |
| 74 | 5.21 |
| 75 | 8.77 |
| 76 | 3.95 |
| 77 | 3.36 |
| 78 | 10.3 |
| 79 | 6.91 |
| 80 | 3.36 |
| 87 | 12.0 |
| 88 | 1.53 |
| 89 | 1.14 |
| 90 | 20.2 |
| 91 | 46.0 |
| 92 | 10.8 |
| 93 | 42.3 |
| 94 | 21.6 |
| 95 | 15.0 |
| 96 | 12.7 |
| 97 | 28.1 |
| 98 | 22.2 |
| 99 | 19.7 |
| 100 | 16.9 |
| 102 | 17.6 |
| 103 | 16.1 |
| 104 | 15.8 |
| 105 | 8.05 |
| 106 | 12.9 |
| 107 | 8.46 |
| 108 | 56.6 |
| 109 | 3.45 |
| 110 | 1.91 |
| 111 | 27.8 |
| 112 | 14.0 |
| 113 | 58.0 |
| 114 | 6.49 |
| 116 | 5.66 |
| 117 | 8.29 |
| 118 | 7.54 |
| 119 | 13.5 |
| 120 | 44.3 |
| 121 | 13.2 |
| 122 | 37.4 |
| 123 | 4.48 |
| 124 | 1.79 |
| 125 | 7.94 |
| 126 | 13.5 |
| 127 | 3.43 |
| 128 | 3.50 |
| 129 | 51.5 |
| 130 | 4.53 |
| 131 | 4.79 |
| 132 | 9.21 |
| 133 | 5.72 |
| 134 | 6.57 |
| 135 | 1.87 |
| 136 | 47.1 |
| 137 | 8.52 |
| 138 | 18.5 |
| 139 | 15.9 |
| 140 | 6.74 |
| 141 | 10.7 |
| 142 | 12.3 |
| 144 | 2.18 |
| 146 | 11.5 |
| 147 | 5.33 |
| 148 | 4.93 |
| 164 | 20.9 |
| 165 | 5.59 |
| 166 | 1.70 |
| 167 | 3.91 |
| 168 | 4.01 |
| 169 | 4.73 |
| 170 | 1.75 |
| 171 | 1.80 |
| 180 | 78.6 |
| 181 | 84.0 |
| 182 | 11.6 |
| 183 | 26.4 |
| 184 | 13.1 |
| 185 | 44.4 |
| 186 | 32.2 |
| 187 | 16.9 |
| 188 | 8.98 |
| 189 | 88.3 |
| 190 | 119 |
| 191 | 30.0 |
| 192 | 77.9 |
| 193 | 74.5 |
| 194 | 70.2 |
| 195 | 63.3 |
| 196 | 16.0 |
| 197 | 46.4 |
| 198 | 45.3 |
| 199 | 26.0 |
| 200 | 16.0 |
| 201 | 10.5 |
| 202 | 90.2 |
| Rimonabant | 5.0 |

As shown in Table 1, the inventive compounds are effective as a cannabinoid CB₁ receptor ligand.

While the invention has been described with respect to the specific embodiments, it should be recognized that various modifications and changes may be made by those skilled in the art to the invention which also fall within the scope of the invention as defined as the appended claims.

What is claimed is:

1. A compound of formula (I) or a pharmaceutically acceptable salt thereof: 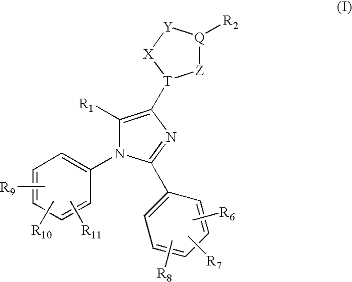
wherein:
R₁ is hydrogen, C₁₋₅ alkyl, substituted C₁₋₅ alkyl, C₂₋₄ alkenyl, substituted C₂₋₄ alkenyl, C₂₋₄ alkynyl, substituted C₂₋₄ alkynyl, halogen, (CH₂)ₙ—C₃₋₅ carbocycle or (CH₂)ₙ-heterocycle, n being 0 or 1;
R₂ is hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, C₃₋₆ carbocycle-R₅ or (CH₂)ₘ—R₅, m being 1 or2;
R₃ and R₄ are each independently hydrogen, C₁₋₆ alkyl, substituted C₁₋₆ alkyl, C₂₋₆ alkenyl, substituted C₂₋₆ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl; or
R₃ and R₄, together with the nitrogen atom to which they are bonded, form a 4- to 10-membered saturated or unsaturated heterocyclic ring which is optionally substituted by one or more C₁₋₃ alkyl, benzyl, phenyl, C₁₋₃ alkoxy or halogen;
R₅ is heteroalkyl, phenyl, furanyl, benzofuranyl, thienyl, benzothienyl, pyridinyl, pyrimidinyl, pyrazinyl, pyridizinyl, tetrahydrofuranyl, tetrahydropyranyl, dioxanyl, 1,4-benzodioxanyl or benzo[1,3]dioxolyl, each being optionally substituted by one or more groups consisting of halogen, C₁₋₃ alkyl and C₁₋₂ alkoxy, each having optional one to three fluorine substituents;
R₆, R₇, R₈, R₉, R₁₀ and R₁₁ are each independently hydrogen, halogen, C₁₋₃ alkyl, C₁₋₃ alkoxy or trifluoromethyl;
X, Y and Z are each independently selected from the group consisting of —C(R₁₂)═, —O—, —N═, —N(R₁₃)— and —S— to form an aromatic heterocycle together with Q and T; 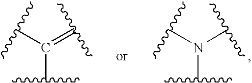
Q and T are each independently
with the proviso that both Q and T can not be simultaneously 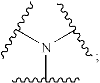
and
R₁₂ and R₁₃ are each independently hydrogen, carbocycle, substituted carbcycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, C₂₋₆ alkynyl optionally substituted by alkoxy or halogen, (CH₂)ₘ—C₃₋₆ carbocycle optionally substituted by alkoxy or halogen, or (CH₂)ₘ—R₅, m being 1 or 2, and R₅ having the same meaning as defined above.

2. The compound or pharmaceutically acceptable salt of claim 1, which is a compound of formula (Ia), (Ib), (Ic) or (Id): 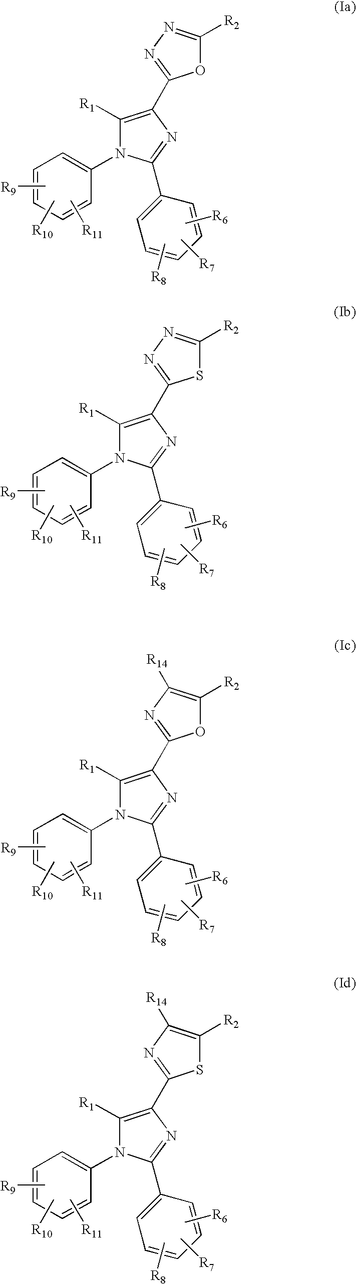
wherein, R₁, R₆, R₇, R₈, R₉, R₁₀ and R₁₁ have the same meanings as defined in claim 1; and
R₂ and R₁₄ are each independently hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₅, m being 1 or 2, or
R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

3. The compound or pharmaceutically acceptable salt of claim 1, which is selected from the group consisting of:
2-tert-butyl-5-(1-(4-chlorophenyl)-2-(2,4-dichlorophenyl)-5-methyl-1H-imidazol-4-yl)-1,3,4-oxadiazole;
2-(1-(4-Chlorophenyl)-2-(2,4-dichlorophenyl)-5-methyl-1H-imidazol-4-yl)-5-cyclohexyl-1,3,4-oxadiazole;
2-(1-(4-Chlorophenyl)-2-(2,4-dichlorophenyl)-5-methyl-1H-imidazol-4-yl)-5-cyclopentyl-1,3,4-oxadiazole;
2-(1-(4-Chlorophenyl)-2-(2,4-dichlorophenyl)-5-methyl-1H-imidazol-4-yl)-5-cyclobutyl-1,3,4-oxadiazole;
2-(1-(4-Chlorophenyl)-2-(2,4-dichlorophenyl)-5-methyl-1H-imidazol-4-yl)-5-cycloheptyl-1,3,4-oxadiazole;
2-(1-(4-Chlorophenyl)-2-(2,4-dichlorophenyl)-5-methyl-1H-imidazol-4-yl)-5-(hexan-2-yl)-1,3,4-oxadiazole;
2-(1-(4-Chlorophenyl)-2-(2,4-dichlorophenyl)-5-methyl-1H-imidazol-4-yl)-5-(pentan-2-yl)-1,3,4-oxadiazole;
2-sec-Butyl-5-(1-(4-chlorophenyl)-2-(2,4-dichlorophenyl)-5-methyl-1H-imidazol-4-yl)-1,3,4-oxadiazole;
2-(1-(4-Chlorophenyl)-2-(2,4-dichlorophenyl)-5-methyl-1H-imidazol-4-yl)-5-isopropyl-1,3,4-oxadiazole;
2-(1-(4-Chlorophenyl)-2-(2,4-dichlorophenyl)-5-methyl-1H-imidazol-4-yl)-5-(pentan-3-yl)-1,3,4-oxadiazole;
2-(1-(4-Chlorophenyl)-2-(2,4-dichlorophenyl)-5-methyl-1H-imidazol-4-yl)-5-(1-phenylethyl)-1,3,4-oxadiazole;
2-(1-(4-Chlorophenyl)-2-(2,4-dichlorophenyl)-5-ethyl-1H-imidazol-4-yl)-5-cyclohexyl-1,3,4-oxadiazole;
2-(1-(4-Chlorophenyl)-2-(2,4-dichlorophenyl)-5-ethyl-1H-imidazol-4-yl)-5-cyclopentyl-1,3,4-oxadiazole;
2-(1-(4-Chlorophenyl)-2-(2,4-dichlorophenyl)-5-ethyl-1H-imidazol-4-yl)-5-cyclobutyl-1,3,4-oxadiazole;
2-tert-butyl-5-(1-(4-chlorophenyl)-2-(2,4-dichlorophenyl)-5-ethyl-1H-imidazol-4-yl)-1,3,4-oxadiazole;
2-(1-(4-Chlorophenyl)-2-(2,4-dichlorophenyl)-5-ethyl-1H-imidazol-4-yl)-5-(hexan-2-yl)-1,3,4-oxadiazole;
2-(1-(4-Chlorophenyl)-2-(2,4-dichlorophenyl)-5-ethyl-1H-imidazol-4-yl)-5-(pentan-2-yl)-1,3,4-oxadiazole;
2-sec-Butyl-5-(1-(4-chlorophenyl)-2-(2,4-dichlorophenyl)-5-ethyl-1H-imidazol-4-yl)-1,3,4-oxadiazole;
2-(1-(4-Bromophenyl)-2-(2,4-dichlorophenyl)-5-methyl-1H-imidazol-4-yl)-5-cyclohexyl-1,3,4-oxadiazole;
2-(1-(4-Bromophenyl)-2-(2,4-dichlorophenyl)-5-methyl-1H-imidazol-4-yl)-5-tert-butyl-1,3,4-oxadiazole;
2-(1-(4-Bromophenyl)-2-(2,4-dichlorophenyl)-5-ethyl-1H-imidazol-4-yl)-5-tert-butyl-1,3,4-oxadiazole;
2-(1-(4-Bromophenyl)-2-(2,4-dichlorophenyl)-5-methyl-1H-imidazol-4-yl)-5-cyclobutyl-1,3,4-oxadiazole;
2-(1-(4-Bromophenyl)-2-(2,4-dichlorophenyl)-5-ethyl-1H-imidazol-4-yl)-5-cyclobutyl-1,3,4-oxadiazole;
2-tert-butyl-5-(1-(4-chlorophenyl)-5-cyclopropyl-2-(2,4-dichlorophenyl)-1H-imidazol-4-yl)-1,3,4-oxadiazole;
2-(1-(4-Bromophenyl)-2-(2,4-dichlorophenyl)-5-isopropyl-1H-imidazol-4-yl)-5-tert-butyl-1,3,4-oxadiazole;
2-(1-(4-Bromophenyl)-2-(2,4-dichlorophenyl)-5-isopropyl-1H-imidazol-4-yl)-5-(1-phenylcyclopropyl)-1,3,4-oxadiazole;
2-(1-(4-Bromophenyl)-2-(2,4-dichlorophenyl)-5-ethyl-1H-imidazol-4-yl)-5-(1-phenylcyclopropyl)-1,3,4-oxadiazole;
2-(1-(4-Chlorophenyl)-2-(2,4-dichlorophenyl)-5-isopropyl-1H-imidazol-4-yl)-5-(1-phenylcyclopropyl)-1,3,4-oxadiazole;
2-(1-(4-Bromophenyl)-2-(2,4-dichlorophenyl)-5-propyl-1H-imidazol-4-yl)-5-tert-butyl-1,3,4-oxadiazole;
2-(1-(4-Bromophenyl)-2-(2,4-dichlorophenyl)-5-propyl-1H-imidazol-4-yl)-5-cyclohexyl-1,3,4-oxadiazole;
2-(1-(4-Bromophenyl)-2-(2,4-dichlorophenyl)-5-propyl-1H-imidazol-4-yl)-5-(1-phenylcyclopropyl)-1,3,4-oxadiazole;
2-(1-(4-Bromophenyl)-2-(2,4-dichlorophenyl)-5-propyl-1H-imidazol-4-yl)-5-cyclobutyl-1,3,4-oxadiazole;
2-(1-(4-Bromophenyl)-2-(2,4-dichlorophenyl)-5-propyl-1H-imidazol-4-yl)-5-(1-(4-chlorophenyl)cyclopropyl)-1,3,4-oxadiazole;
2-(1-(4-Bromophenyl)-2-(2,4-dichlorophenyl)-5-propyl-1H-imidazol-4-yl)-5-(1-(4-chlorophenyl)cyclobutyl)-1,3,4-oxadiazole;
2-tert-butyl-5-(1-(4-chlorophenyl)-2-(2,4-dichlorophenyl)-5-propyl-1H-imidazol-4-yl)-1,3,4-oxadiazole;
2-(1-(4-Chlorophenyl)-2-(2,4-dichlorophenyl)-5-propyl-1H-imidazol-4-yl)-5-cyclobutyl-1,3,4-oxadiazole;
2-(1-(4-Chlorophenyl)-2-(2,4-dichlorophenyl)-5-propyl-1H-imidazol-4-yl)-5-(1-(4-chlorophenyl)cyclopropyl)-1,3,4-oxadiazole;
2-(1-(4-Chlorophenyl)-2-(2,4-dichlorophenyl)-5-propyl-1H-imidazol-4-yl)-5-(1-(4-chlorophenyl)cyclobutyl)-1,3,4-oxadiazole;
2-(1-(4-Chlorophenyl)-2-(2,4-dichlorophenyl)-5-propyl-1H-imidazol-4-yl)-5-cyclohexyl-1,3,4-oxadiazole;
2-(1-(4-Bromophenyl)-2-(2,4-dichlorophenyl)-5-ethyl-1H-imidazol-4-yl)-5-(1-(4-chlorophenyl)cyclopropyl)-1,3,4-oxadiazole;
2-(1-(4-Bromophenyl)-2-(2,4-dichlorophenyl)-5-ethyl-1H-imidazol-4-yl)-5-(1-(4-chlorophenyl)cyclobutyl)-1,3,4-oxadiazole;
2-(1-(4-Bromophenyl)-2-(2,4-dichlorophenyl)-5-ethyl-1H-imidazol-4-yl)-5-(1-(4-methoxyphenyl)cyclopropyl)-1,3,4-oxadiazole;
2-(1-(4-Bromophenyl)-2-(2,4-dichlorophenyl)-5-ethyl-1H-imidazol-4-yl)-5-(1-p-tolylcyclopropyl)-1,3,4-oxadiazole;
2-(1-(4-Bromophenyl)-2-(2,4-dichlorophenyl)-5-ethyl-1H-imidazol-4-yl)-5-(1-(2,4-dichlorophenyl)cyclopropyl)-1,3,4-oxadiazole;
2-(1-(4-Bromophenyl)-2-(2,4-dichlorophenyl)-5-ethyl-1H-imidazol-4-yl)-5-(1-(4-chlorophenyl)cyclopentyl)-1,3,4-oxadiazole;
2-(1-(4-Bromophenyl)-2-(2,4-dichlorophenyl)-5-methyl-1H-imidazol-4-yl)-5-(1-(trifluoromethyl)cyclopropyl)-1,3,4-oxadiazole;
2-(1-(4-Bromophenyl)-2-(2,4-dichlorophenyl)-5-methyl-1H-imidazol-4-yl)-5-(1-(trifluoromethyl)cyclobutyl)-1,3,4-oxadiazole;
2-(1-(4-Bromophenyl)-2-(2,4-dichlorophenyl)-5-methyl-1H-imidazol-4-yl)-5-(1-(4-chlorophenyl)cyclopropyl)-1,3,4-oxadiazole;
2-(1-(4-Bromophenyl)-2-(2,4-dichlorophenyl)-5-ethyl-1H-imidazol-4-yl)-5-(1-(trifluoromethyl)cyclopropyl)-1,3,4-oxadiazole;
2-(1-(4-Bromophenyl)-2-(2,4-dichlorophenyl)-5-ethyl-1H-imidazol-4-yl)-5-(1-(trifluoromethyl)cyclobutyl)-1,3,4-oxadiazole;
2-(1-(4-Bromophenyl)-2-(2-chlorophenyl)-5-methyl-1H-imidazol-4-yl)-5-tert-butyl-1,3,4-oxadiazole;
2-(1-(4-Bromophenyl)-2-(2-chlorophenyl)-5-methyl-1H-imidazol-4-yl)-5-(1-(trifluoromethyl)cyclopropyl)-1,3,4-oxadiazole;
2-(1-(4-Bromophenyl)-2-(2-chlorophenyl)-5-methyl-1H-imidazol-4-yl)-5-(1-(trifluoromethyl)cyclobutyl)-1,3,4-oxadiazole;
2-(1-(4-Bromophenyl)-2-(2-chlorophenyl)-5-methyl-1H-imidazol-4-yl)-5-(1-(4-chlorophenyl)cyclopropyl)-1,3,4-oxadiazole;
2-(1-(4-Bromophenyl)-2-(2-chlorophenyl)-5-ethyl-1H-imidazol-4-yl)-5-tert-butyl-1,3,4-oxadiazole;
2-(1-(4-Bromophenyl)-2-(2-chlorophenyl)-5-ethyl-1H-imidazol-4-yl)-5-(1-(trifluoromethyl)cyclopropyl)-1,3,4-oxadiazole;
2-(1-(4-Bromophenyl)-2-(2-chlorophenyl)-5-ethyl-1H-imidazol-4-yl)-5-(1-(trifluoromethyl)cyclobutyl)-1,3,4-oxadiazole;
2-(1-(4-Bromophenyl)-2-(2-chlorophenyl)-5-ethyl-1H-imidazol-4-yl)-5-(1-(4-chlorophenyl)cyclopropyl)-1,3,4-oxadiazole;
2-tert-Butyl-5-(2-(2-chlorophenyl)-1-(4-chlorophenyl)-5-methyl-1H-imidazol-4-yl)-1,3,4-oxadiazole;
2-(2-(2-Chlorophenyl)-1-(4-chlorophenyl)-5-methyl-1H-imidazol-4-yl)-5-(1-(trifluoromethyl)cyclopropyl)-1,3,4-oxadiazole;
2-(2-(2-Chlorophenyl)-1-(4-chlorophenyl)-5-methyl-1H-imidazol-4-yl)-5-(1-(trifluoromethyl)cyclobutyl)-1,3,4-oxadiazole;
2-(2-(2-Chlorophenyl)-1-(4-chlorophenyl)-5-methyl-1H-imidazol-4-yl)-5-(1-(4-chlorophenyl)cyclopropyl)-1,3,4-oxadiazole;
2-tert-Butyl-5-(2-(2-chlorophenyl)-1-(4-chlorophenyl)-5-ethyl-1H-imidazol-4-yl)-1,3,4-oxadiazole;
2-(2-(2-Chlorophenyl)-1-(4-chlorophenyl)-5-ethyl-1H-imidazol-4-yl)-5-(1-(trifluoromethyl)cyclopropyl)-1,3,4-oxadiazole;
2-(2-(2-Chlorophenyl)-1-(4-chlorophenyl)-5-ethyl-1H-imidazol-4-yl)-5-(1-(trifluoromethyl)cyclobutyl)-1,3,4-oxadiazole;
2-(2-(2-Chlorophenyl)-1-(4-chlorophenyl)-5-ethyl-1H-imidazol-4-yl)-5-(1-(4-chlorophenyl)cyclopropyl)-1,3,4-oxadiazole;
2-(1-(4-Chlorophenyl)-2-(2,4-dichlorophenyl)-5-methyl-1H-imidazol-4-yl)-5-1-(trifluoromethyl)cyclopropyl)-1,3,4-oxadiazole;
2-(1-(4-Chlorophenyl)-2-(2,4-dichlorophenyl)-5-methyl-1H-imidazol-4-yl)-5-(1-(trifluoromethyl)cyclobutyl)-1,3,4-oxadiazole;
2-(1-(4-Chlorophenyl)-2-(2,4-dichlorophenyl)-5-methyl-1H-imidazol-4-yl)-5-(1-(4-chlorophenyl)cyclopropyl)-1,3,4-oxadiazole;
2-(1-(4-Chlorophenyl)-2-(2,4-dichlorophenyl)-5-ethyl-1H-imidazol-4-yl)-5-(1-(trifluoromethyl)cyclopropyl)-1,3,4-oxadiazole;
2-(1-(4-Chlorophenyl)-2-(2,4-dichlorophenyl)-5-ethyl-1H-imidazol-4-yl)-5-(1-(trifluoromethyl)cyclobutyl)-1,3,4-oxadiazole;
2-(1-(4-Chlorophenyl)-2-(2,4-dichlorophenyl)-5-ethyl-1H-imidazol-4-yl)-5-(1-(4-chlorophenyl)cyclopropyl)-1,3,4-oxadiazole;
2-(1-(4-Chlorophenyl)-2-(2,4-dichlorophenyl)-5-ethyl-1H-imidazol-4-yl)-5-(trifluoromethyl)-1,3,4-oxadiazole;
2-(5-((l H-1,2,4-Triazol-1-yl)methyl)-1-(4-bromophenyl)-2-(2,4-dichlorophenyl)-1H-imidazol-4-yl)-5-tert-butyl-1,3,4-oxadiazole;
2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclopropyl)-1,3,4-oxadiazole;
2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclobutyl)-1,3,4-oxadiazole;
2-(5-((1H-1,2,4-Triazol-1-yl)methyl)-1-(4-bromophenyl)-2-(2,4-dichlorophenyl)-1H-imidazol-4-yl)-5-(1-(4-chlorophenyl)cyclopropyl)-1,3,4-oxadiazole;
2-(5-((1H-1,2,4-Triazol-1-yl)methyl)-1-(4-bromophenyl)-2-(2-chlorophenyl)-1H-imidazol-4-yl)-5-tert-butyl-1,3,4-oxadiazole;
2-(5-((1H-1,2,4-Triazol-1-yl)methyl)-1-(4-bromophenyl)-2-(2-chlorophenyl)-1H-imidazol-4-yl)-5-(1-(trifluoromethyl)cyclopropyl)-1,3,4-oxadiazole;
2-(5-((1H-1,2,4-Triazol-1-yl)methyl)-1-(4-bromophenyl)-2-(2-chlorophenyl)-1H-imidazol-4-yl)-5-(1-(trifluoromethyl)cyclobutyl)-1,3,4-oxadiazole;
2-(5-((1H-1,2,4-Triazol-1-yl)methyl)-1-(4-bromophenyl)-2-(2-chlorophenyl)-1H-imidazol-4-yl)-5-(1-(4-chlorophenyl)cyclopropyl)-1,3,4-oxadiazole;
2-(5-((1H-1,2,4-Triazol-1-yl)methyl)-2-(2-chlorophenyl)-1-(4-chlorophenyl)-1H-imidazol-4-yl)-5-tert-butyl-1,3,4-oxadiazole;
2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclopropyl)-1,3,4-oxadiazole;
2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclobutyl)-1,3,4-oxadiazole;
2-(5-((1H-1,2,4-Triazol-1-yl)methyl)-2-(2-chlorophenyl)-1-(4-chlorophenyl)-1H-imidazol-4-yl)-5-(1-(4-chlorophenyl)cyclopropyl)-1,3,4-oxadiazole;
2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclobutyl)-1,3,4-oxadiazole;
2-(5-((1H-1,2,4-Triazol-1-yl)methyl)-1-(4-chlorophenyl)-2-(2,4-dichlorophenyl)-1H-imidazol-4-yl)-5-tert-butyl-1,3,4-oxadiazole;
2-(5-((1H-1,2,4-Triazol-1-yl)methyl)-1-(4-chlorophenyl)-2-(2,4-dichlorophenyl)-1H-imidazol-4-yl)-5-(1-(4-chlorophenyl)cyclopropyl)-1,3,4-oxadiazole;
2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclopropyl)-1,3,4-oxadiazole;
2-tert-butyl-5-(1-(4-chlorophenyl)-2-(2,4-dichlorophenyl)-5-methyl-1H-imidazol-4-yl)-1,3,4-thiadiazole;
2-(1-(4-Chlorophenyl)-2-(2,4-dichlorophenyl)-5-methyl-1H-imidazol-4-yl)-5-cyclohexyl-1,3,4-thiadiazole;
2-(1-(4-Chlorophenyl)-2-(2,4-dichlorophenyl)-5-methyl-1H-imidazol-4-yl)-5-cyclopentyl-1,3,4-thiadiazole;
2-(1-(4-Chlorophenyl)-2-(2,4-dichlorophenyl)-5-methyl-1H-imidazol-4-yl)-5-cyclobutyl-1,3,4-thiadiazole;
2-(1-(4-Chlorophenyl)-2-(2,4-dichlorophenyl)-5-methyl-1H-imidazol-4-yl)-5-cycloheptyl-1,3,4-thiadiazole;
2-(1-(4-Chlorophenyl)-2-(2,4-dichlorophenyl)-5-methyl-1H-imidazol-4-yl)-5-(hexan-2-yl)-1,3,4-thiadiazole;
2-(1-(4-Chlorophenyl)-2-(2,4-dichlorophenyl)-5-methyl-1H-imidazol-4-yl)-5(-pentan-2-yl)-1,3,4-thiadiazole;
2-sec-Butyl-5-(1-(4-chlorophenyl)-2-(2,4-dichlorophenyl)-5-methyl-1H-imidazol-4-yl)-1,3,4-thiadiazole;
2-(1-(4-Chlorophenyl)-2-(2,4-dichlorophenyl)-5-methyl-1H-imidazol-4-yl)-5-isopropyl-1,3,4-thiadiazole;
2-(1-(4-Chlorophenyl)-2-(2,4-dichlorophenyl)-5-methyl-1H-imidazol-4-yl)-5-(pentan-3-yl)-1,3,4-thiadiazole;
2-(1-(4-Chlorophenyl)-2-(2,4-dichlorophenyl)-5-methyl-1H-imidazol-4-yl)-5-( 1-phenylethyl)-1,3,4-thiadiazole;
2-(1-(4-Chlorophenyl)-2-(2,4-dichlorophenyl)-5-ethyl-1H-imidazol-4-yl)-5-cyclohexyl-1,3,4-thiadiazole;
2-(1-(4-Chlorophenyl)-2-(2,4-dichlorophenyl)-5-ethyl-1H-imidazol-4-yl)-5-cyclopentyl-1,3,4-thiadiazole;
2-(1-(4-Chlorophenyl)-2-(2,4-dichlorophenyl)-5-ethyl-1H-imidazol-4-yl)-5-cyclobutyl-1,3,4-thiadiazole;
2-tert-Butyl-5-(1-(4-chlorophenyl)-2-(2,4-dichlorophenyl)-5-ethyl-1H-imidazol-4-yl)-1,3,4-thiadiazole;
2-(1-(4-Chlorophenyl)-2-(2,4-dichlorophenyl)-5-ethyl-1H-imidazol-4-yl)-5-(hexan-2-yl)-1,3,4-thiadiazole;
2-(1-(4-Chlorophenyl)-2-(2,4-dichlorophenyl)-5-ethyl-1H-imidazol-4-yl)-5-(pentan-2-yl)-1,3,4-thiadiazole;
2-sec-Butyl-5-(1-(4-chlorophenyl)-2-(2,4-dichlorophenyl)-5-ethyl-1H-imidazol-4-yl)-1,3,4-thiadiazole;
2-(1-(4-Bromophenyl)-2-(2,4-dichlorophenyl)-5-methyl-1H-imidazol-4-yl)-5-cyclohexyl-1,3,4-thiadiazole;
2-(1-(4-Bromophenyl)-2-(2,4-dichlorophenyl)-5-methyl-1H-imidazol-4-yl)-5-tert-butyl-1,3,4-thiadiazole;
2-(1-(4-Bromophenyl)-2-(2,4-dichlorophenyl)-5-ethyl-1H-imidazol-4-yl)-5-tert-butyl-butyl-1,3,4-thiadiazole;
2-(1-(4-Bromophenyl)-2-(2,4-dichlorophenyl)-5-methyl-1H-imidazol-4-yl)-5-cyclobutyl-1,3,4-thiadiazole;
2-(1-(4-Bromophenyl)-2-(2,4-dichlorophenyl)-5-ethyl-1H-imidazol-4-yl)-5-cyclobutyl-1,3,4-thiadiazole;
2-tert-Butyl-5-(1-(4-chlorophenyl)-5-cyclopropyl-2-(2,4-dichlorophenyl)-1H-imidazol-4-yl)-1,3,4-thiadiazole;
2-(1-(4-Bromophenyl)-2-(2,4-dichlorophenyl)-5-isopropyl-1H-imidazol-4-yl)-5-tert-butyl-1,3,4-thiadiazole;
2-(1-(4-Bromophenyl)-2-(2,4-dichlorophenyl)-5-isopropyl-1H-imidazol-4-yl)-5-cyclobutyl-1,3,4-thiadiazole;
2-(1-(4-Bromophenyl)-2-(2,4-dichlorophenyl)-5-isopropyl-1H-imidazol-4-yl)-5-(1-phenylcyclopropyl)-1,3,4-thiadiazole;
2-(1-(4-Bromophenyl)-2-(2,4-dichlorophenyl)-5-ethyl-1H-imidazol-4-yl)-5-(1-phenylcyclopropyl)-1,3,4-thiadiazole;
2-(1-(4-Chlorophenyl)-2-(2,4-dichlorophenyl)-5-isopropyl-1H-imidazol-4-yl)-5-(1-phenylcyclopropyl)-1,3,4-thiadiazole;
2-(1-(4-Bromophenyl)-2-(2,4-dichlorophenyl)-5-propyl-1H-imidazol-4-yl)-5-tert-butyl-1,3,4-thiadiazole;
2-(1-(4-Bromophenyl)-2-(2,4-dichlorophenyl)-5-propyl-1H-imidazol-4-yl)-5-cyclohexyl-1,3,4-thiadiazole;
2-(1-(4-Bromophenyl)-2-(2,4-dichlorophenyl)-5-propyl-1H-imidazol-4-yl)-5-(1-phenylcyclopropyl)-1,3,4-thiadiazole;
2-(1-(4-Bromophenyl)-2-(2,4-dichlorophenyl)-5-propyl-1H-imidazol-4-yl)-5-cyclobutyl-1,3,4-thiadiazole;
2-(1-(4-Bromophenyl)-2-(2,4-dichlorophenyl)-5-propyl-1H-imidazol-4-yl)-5-(1-(4-chlorophenyl)cyclopropyl)-1,3,4-thiadiazole;
2-(1-(4-Bromophenyl)-2-(2,4-dichlorophenyl)-5-propyl-1H-imidazol-4-yl)-5-(1-(4-chlorophenyl)cyclobutyl)-1,3,4-thiadiazole;
2-tert-Butyl-5-(1-(4-chlorophenyl)-2-(2,4-dichlorophenyl)-5-propyl-1H-imidazol-4-yl)-1,3,4-thiadiazole;
2-(1-(4-Chlorophenyl)-2-(2,4-dichlorophenyl)-5-propyl-1H-imidazol-4-yl)-5-cyclobutyl-1,3,4-thiadiazole;
2-(1-(4-Chlorophenyl)-2-(2,4-dichlorophenyl)-5-propyl-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-propyl-1H-imidazol-4-yl)-5-(1-(4-chlorophenyl)cyclobutyl)-1,3,4-thiadiazole;
2-(1-(4-Chlorophenyl)-2-(2,4-dichlorophenyl)-5-propyl-1H-imidazol-4-yl)-5-cyclohexyl-1,3,4-thiadiazole;
2-1-(4-Bromophenyl)-2-(2,4-dichlorophenyl)-5-ethyl-1H-imidazol-4-yl)-5-(1-(4-chlorophenyl)cyclopropyl)-1,3,4-thiadiazole;
2-(1-(4-Bromophenyl)-2-(2,4-dichlorophenyl)-5-ethyl-1H-imidazol-4-yl)-5-(1-(4-chlorophenyl)cyclobutyl)-1,3,4-thiadiazole;
2-(1-(4-Bromophenyl)-2-(2,4-dichlorophenyl)-5-ethyl-1H-imidazol-4-yl)-5-(1-(4-methoxyphenyl)cyclopropyl)-1,3,4-thiadiazole;
2-(1-(4-Bromophenyl)-2-(2,4-dichlorophenyl)-5-ethyl-1H-imidazol-4-yl)-5-(1-p-tolylcyclopropyl)-1,3,4-thiadiazole;
2-(1-(4-Bromophenyl)-2-(2,4-dichlorophenyl)-5-ethyl-1H-imidazol-4-yl)-5-(1-(2,4-dichlorophenyl)cyclopropyl)-1,3,4-thiadiazole;
2-(1-(4-Bromophenyl)-2-(2,4-dichlorophenyl)-5-ethyl-1H-imidazol-4-yl)-5-(1-(4-chlorophenyl)cyclopentyl)-1,3,4-thiadiazole;
2-(1-(4-Bromophenyl)-2-(2,4-dichlorophenyl)-5-methyl-1H-imidazol-4-yl)-5-(1-(trifluoromethyl)cyclopropyl)-1,3,4-thiadiazole;
2-(1-(4-Bromophenyl)-2-(2,4-dichlorophenyl)-5-methyl-1H-imidazol-4-yl)-5-(1-(trifluoromethyl)cyclobutyl)-1,3,4-thiadiazole;
2-(1-(4-Bromophenyl)-2-(2,4-dichlorophenyl)-5-methyl-1H-imidazol-4-yl)-5-(1-(4-chlorophenyl)cyclopropyl)-1,3,4-thiadiazole;
2-(1-(4-Bromophenyl)-2-(2,4-dichlorophenyl)-5-ethyl-1H-imidazol-4-yl)-5-(1-(trifluoromethyl)cyclopropyl)-1,3,4-thiadiazole;
2-(1-(4-Bromophenyl)-2-(2,4-dichlorophenyl)-5-ethyl-1H-imidazol-4-yl)-5-(1-(trifluoromethyl)cyclobutyl)-1,3,4-thiadiazole;
2-(1-(4-Bromophenyl)-2-(2-chlorophenyl)-5-methyl-1H-imidazol-4-yl)-5-tert-butyl-1,3,4-thiadiazole;
2-(1-(4-Bromophenyl)-2-(2-chlorophenyl)-5-methyl-1H-imidazol-4-yl)-5-(1-(trifluoromethyl)cyclopropyl)-1,3,4-thiadiazole;
2-(1-(4-Bromophenyl)-2-(2-chlorophenyl)-5-methyl-1H-imidazol-4-yl)-5-(1-(trifluoromethyl)cyclobutyl)-1,3,4-thiadiazole;
2-(1-(4-Bromophenyl)-2-(2-chlorophenyl)-5-methyl-1H-imidazol-4-yl)-5-(1-(4-chlorophenyl)cyclopropyl)-1,3,4-thiadiazole;
2-(1-(4-Bromophenyl)-2-(2-chlorophenyl)-5-ethyl-1H-imidazol-4-yl)-5-tert-butyl-1,3,4-thiadiazole;
2-(1-(4-Bromophenyl)-2-(2-chlorophenyl)-5-ethyl-1H-imidazol-4-yl)-5-(1-(trifluoromethyl)cyclopropyl)-1,3,4-thiadiazole;
2-(1-(4-Bromophenyl)-2-(2-chlorophenyl)-5-ethyl-1H-imidazol-4-yl)-5-(1-(trifluoromethyl)cyclobutyl)-1,3,4-thiadiazole;
2-(1-(4-Bromophenyl)-2-(2-chlorophenyl)-5-ethyl-1H-imidazol-4-yl)-5-(1-(4-chlorophenyl)cyclopropyl)-1,3,4-thiadiazole;
2-tert-Butyl-5-(2-(2-chlorophenyl)-1-(4-chlorophenyl)-5-methyl-1H-imidazol-4-yl)-1,3,4-thiadiazole;
2-(2-(2-Chlorophenyl)-1-(4-chlorophenyl)-5-methyl-1H-imidazol-4-yl)-5-(1-(trifluoromethyl)cyclopropyl)-1,3,4-thiadiazole;
2-(2-(2-Chlorophenyl)-1-(4-chlorophenyl)-5-methyl-1H-imidazol-4-yl)-5-(1-(trifluoromethyl)cyclobutyl)-1,3,4-thiadiazole;
2-(2-(2-Chlorophenyl)-1-(4-chlorophenyl)-5-methyl-1H-imidazol-4-yl)-5-(1-(4-chlorophenyl)cyclopropyl)-1,3,4-thiadiazole;
2-tert-Butyl-5-(2-(2-chlorophenyl)-1-(4-chlorophenyl)-5-ethyl-1H-imidazol-4-yl)-1,3,4-thiadiazole;
2-(2-(2-Chlorophenyl)-1-(4-chlorophenyl)-5-ethyl-1H-imidazol-4-yl)-5-(1-(trifluoromethyl)cyclopropyl)-1,3,4-thiadiazole;
2-(2-(2-Chlorophenyl)-1-(4-chlorophenyl)-5-ethyl-1H-imidazol-4-yl)-5-(1-(trifluoromethyl)cyclobutyl)-1,3,4-thiadiazole;
2-(2-(2-Chlorophenyl)-1-(4-chlorophenyl)-5-ethyl-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-methyl-1H-imidazol-4-yl)-5-(1-(trifluoromethyl)cyclopropyl)-1,3,4-thiadiazole;
2-(1-(4-Chlorophenyl)-2-(2,4-dichlorophenyl)-5-methyl-1H-imidazol-4-yl)-5-(1-(trifluoromethyl)cyclobutyl)-1,3,4-thiadiazole;
2-(1-(4-Chlorophenyl)-2-(2,4-dichlorophenyl)-5-methyl-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-ethyl-1H-imidazol-4-yl)-5-(1-(trifluoromethyl)cyclopropyl)-1,3,4-thiadiazole;
2-(1-(4-Chlorophenyl)-2-(2,4-dichlorophenyl)-5-ethyl-1H-imidazol-4-yl)-5-(1-(trifluoromethyl)cyclobutyl)-1,3,4-thiadiazole;
2-(1-(4-Chlorophenyl)-2-(2,4-dichlorophenyl)-5-ethyl-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-ethyl-1H-imidazol-4-yl)-5-(trifluoromethyl)-1,3,4-thiadiazole;
2-(5-((1H-1,2,4-Triazol-1-yl)methyl)-1-(4-bromophenyl)-2-(2,4-dichlorophenyl)-1H-imidazol-4-yl)-5-tert-butyl-1,3,4-thiadiazole;
2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclopropyl)-1,3,4-thiadiazole;
2-(5-((1H-1,2,4-Triazol-1-yl)methyl)-1-(4-bromophenyl)-2-(2,4-dichlorophenyl)-1H-imidazol-4-yl)-5-(1-(trifluoromethyl)cyclobutyl)-1,3,4-thiadiazole;
2-(5-((1H-1,2,4-Triazol-1-yl)methyl)-1-(4-bromophenyl)-2-(2,4-dichlorophenyl)-1H-imidazol-4-yl)-5-(1-(4-chlorophenyl)cyclopropyl)-1,3,4-thiadiazole;
2-(5-((1H-1,2,4-Triazol-1-yl)methyl)-1-(4-bromophenyl)-2-(2-chlorophenyl)-1H-imidazol-4-yl)-5-tert-butyl-1,3,4-thiadiazole;
2-(5-((1H-1,2,4-Triazol-1-yl)methyl)-1-(4-bromophenyl)-2-(2-chlorophenyl)-1H-imidazol-4-yl)-5-(1-(trifluoromethyl)cyclopropyl)-1,3,4-thiadiazole;
2-(5-((1H-1,2,4-Triazol-1-yl)methyl)-1-(4-bromophenyl)-2-(2-chlorophenyl)-1H-imidazol-4-yl)-5-(1-(trifluoromethyl)cyclobutyl)-1,3,4-thiadiazole;
2-(5-((1H-1,2,4-Triazol-1-yl)methyl)-1-(4-bromophenyl)-2-(2-chlorophenyl)-1H-imidazol-4-yl)-5-(1-(4-chlorophenyl)cyclopropyl)-1,3,4-thiadiazole;
2-(5-((1H-1,2,4-Triazol-1-yl)methyl)-2-(2-chlorophenyl)-1-(4-chlorophenyl)-1H-imidazol-4-yl)-5-tert-butyl-1,3,4-thiadiazole;
2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclopropyl)-1,3,4-thiadiazole;
2-(5-((1H-1,2,4-Triazol-1-yl)methyl)-2-(2-chlorophenyl)-1-(4-chlorophenyl)-1H-imidazol-4-yl)-5-(1-(trifluoromethyl)cyclobutyl)-1,3,4-thiadiazole;
2-(5-((1H-1,2,4-Triazol-1-yl)methyl)-2-(2-chlorophenyl)-1-(4-chlorophenyl)-1H-imidazol-4-yl)-5-(1-(4-chlorophenyl)cyclopropyl)-1,3,4-thiadiazole;
2-(5-((1H-1,2,4-Triazol-1-yl)methyl)-1-(4-chlorophenyl)-2-(2,4-dichlorophenyl)-1H-imidazol-4-yl)-5-tert-butyl-1,3,4-thiadiazole;
2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclopropyl)-1,3,4-thiadiazole;
2-(5-((1H-1,2,4-Triazol-1-yl)methyl)-1-(4-chlorophenyl)-2-(2,4-dichlorophenyl)-1H-imidazol-4-yl)-5-(1-(trifluoromethyl)cyclobutyl)-1,3,4-thiadiazole;
2-(5-((1H-1,2,4-Triazol-1-yl)methyl)-1-(4-chlorophenyl)-2-(2,4-dichlorophenyl)-1H-imidazol-4-yl)-5-(1-(4-chlorophenyl)cyclopropyl)-1,3,4-thiadiazole;
2-(1-(4-Chlorophenyl)-2-(2,4-dichlorophenyl)-5-methyl-1H-imidazol-4-yl)-5-cyclohexyloxazole;
2-(1-(4-Chlorophenyl)-2-(2,4-dichlorophenyl)-5-methyl-1H-imidazol-4-yl)-5-ethyloxazole;
5-Butyl-2-(1-(4-chlorophenyl)-2-(2,4-dichlorophenyl)-5-methyl-1H-imidazol-4-yl)oxazole;
2-(1-(4-Chlorophenyl)-2-(2,4-dichlorophenyl)-5-methyl-1H-imidazol-4-yl)-5-isopropyloxazole;
2-(1-(4-Chlorophenyl)-2-(2,4-dichlorophenyl)-5-methyl-1H-imidazol-4-yl)-5-isobutyloxazole;
2-tert-Butyl-5-(1-(4-chlorophenyl)-2-(2,4-dichlorophenyl)-5-methyl-1H-imidazol-4-yl)oxazole;
2-tert-Butyl-5-(1-(4-chlorophenyl)-2-(2,4-dichlorophenyl)-5-ethyl-1H-imidazol-4-yl)oxazole;
2-(1-(4-Bromophenyl)-2-(2,4-dichlorophenyl)-5-methyl-1H-imidazol-4-yl)-5-tert-butyloxazole;
2-(1-(4-Bromophenyl)-2-(2,4-dichlorophenyl)-5-ethyl-1H-imidazol-4-yl)-5-tert-butyloxazole;
4-tert-Butyl-2-(1-(4-chlorophenyl)-2-(2,4-dichlorophenyl)-5-methyl-1H-imidazol-4-yl)oxazole;
4-tert-Butyl-2-(1-(4-chlorophenyl)-2-(2,4-dichlorophenyl)-5-ethyl-1H-imidazol-4-yl)oxazole;
2-(1-(4-Bromophenyl)-2-(2,4-dichlorophenyl)-5-ethyl-1H-imidazol-4-yl)-4-tert-butyloxazole;
2-(1-(4-Chlorophenyl)-2-(2,4-dichlorophenyl)-5-methyl-1H-imidazol-4-yl)-5-ethyl-4-methyloxazole;
2-(1-(4-Chlorophenyl)-2-(2,4-dichlorophenyl)-5-methyl-1H-imidazol-4-yl)-5-cyclohexylthiazole;
2-(1-(4-Chlorophenyl)-2-(2,4-dichlorophenyl)-5-methyl-1H-imidazol-4-yl)-5-ethylthiazole;
5-Butyl-2-(1-(4-chlorophenyl)-2-(2,4-dichlorophenyl)-5-methyl-1H-imidazol-4-yl)thiazole;
2-(1-(4-Chlorophenyl)-2-(2,4-dichlorophenyl)-5-methyl-1H-imidazol-4-yl)-5-isopropylthiazole;
2-(1-(4-Chlorophenyl)-2-(2,4-dichlorophenyl)-5-methyl-1H-imidazol-4-yl)-5-isobutylthiazole;
2-tert-Butyl-5-(1-(4-chlorophenyl)-2-(2,4-dichlorophenyl)-5-methyl-1H-imidazol-4-yl)thiazole;
2-tert-Butyl-5-(1-(4-chlorophenyl)-2-(2,4-dichlorophenyl)-5-ethyl-1H-imidazol-4-yl)thiazole;
2-(1-(4-Bromophenyl)-2-(2,4-dichlorophenyl)-5-methyl-1H-imidazol-4-yl)-5-tert-butylthiazole;
2-(1-(4-Bromophenyl)-2-(2,4-dichlorophenyl)-5-ethyl-1H-imidazol-4-yl)-5-tert-butylthiazole; and
2-(1-(4-Chlorophenyl)-2-(2,4-dichlorophenyl)-5-methyl-1H-imidazol-4-yl)-5-ethyl-4-methylthiazole.

4. A method for preparing the compound of formula (Ia) of claim 2, which comprises (i) reacting a carboxylic acid derivative of formula (5) with a hydrazide compound of formula (7) in the presence of a coupling reagent in a solvent, and (ii) cyclizing the resulting product using a dehydrating agent: 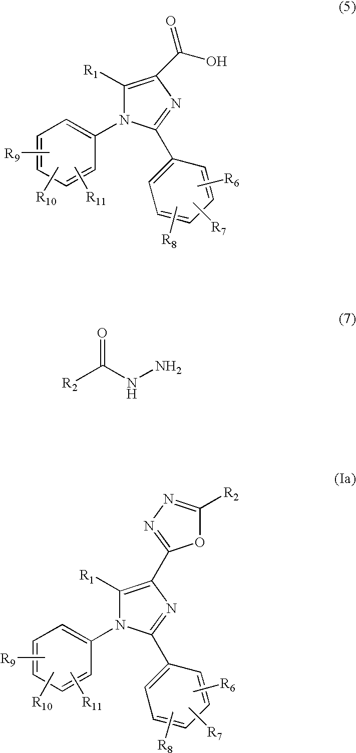
wherein, R₁, R₂, R₆, R₇, R₈, R₉, R₁₀ and R₁₁ have the same meanings as defined in claim 1.

5. The method of claim 4, wherein the dehydrating agent used in (ii) is Burgess reagent.

6. A method for preparing the compound of formula (Ib) of claim 2, which comprises (i) reacting a carboxylic acid derivative of formula (5) with a hydrazide compound of formula (7) in the presence of a coupling reagent in a solvent and (ii) cyclizing the resulting product using Lawesson's reagent: 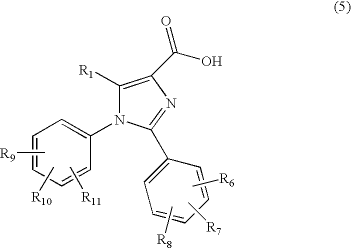 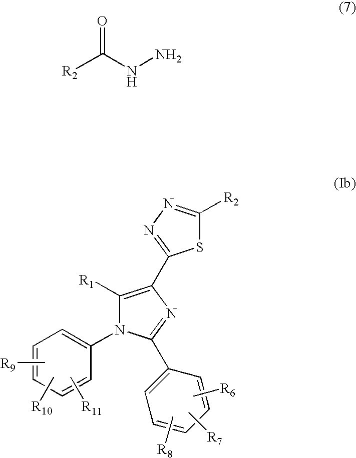
wherein, R₁, R₂, R₆, R₇, R₈, R₉, R₁₀ and R₁₁ have the same meanings as defined in claim 1.

7. A method for preparing the compound of formula (Ic) of claim 2, which comprises (i) reacting a carboxylic acid derivative of formula (5) with a salt of an aminoketone compound of formula (10) in the presence of a coupling reagent in a solvent, and (ii) cyclizing the resulting product using a dehydrating agent: 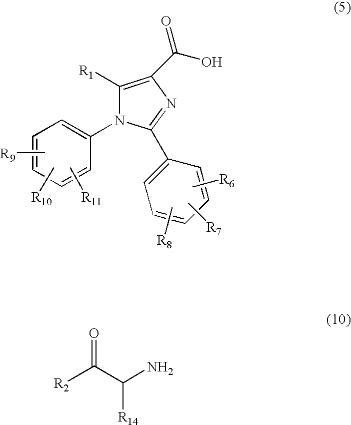 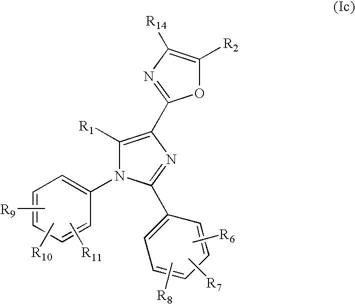
wherein, R₁, R₆, R₇, R₈, R₉, R₁₀ and R₁₁ have the same meanings as defined in claim 1; and
R₂ and R₁₄ are each independently hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₅, m being 1 or 2, or
R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

8. The method of claim 7, wherein the dehydrating agent used in (ii) is Burgess reagent.

9. A method for preparing the compound of formula (Id) of claim 2, which comprises (i) reacting a carboxylic acid derivative of formula (5) with a salt of an aminoketone compound of formula (10) in the presence of a coupling reagent in a solvent and (ii) cyclizing the resulting product using Lawesson's reagent: 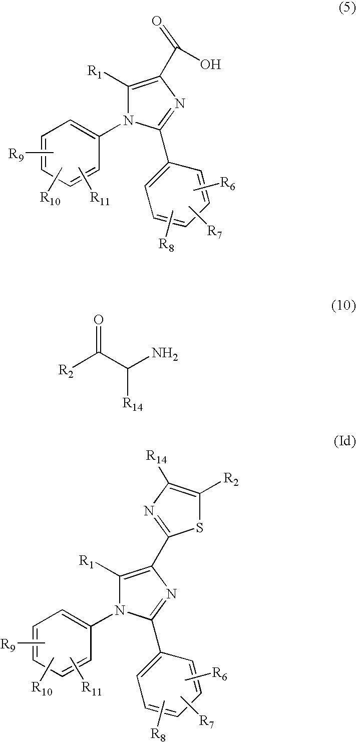
wherein, R₁, R₂, R₆, R₇, R₈, R₉, R₁₀ and R₁₁ have the same meanings as defined in claim 1; and
R₂ and R₁₄ are each independently hydrogen, NR₃R₄, carbocycle, substituted carbocycle, aryl, substituted aryl, heteroaryl, substituted heteroaryl, heterocycle, substituted heterocycle, C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₅, m being 1 or2, or
R₂ and R₁₄ are bonded together to form a 4- to 10-membered saturated or unsaturated carbocyclic or heterocyclic ring which is optionally substituted by one or more C₁₋₃ alkyl, benzyl, phenyl, C₁₋₃ alkoxy or halogen.

10. A pharmaceutical composition comprising the compound of formula (I) of claim 1 as an active ingredient and a pharmaceutically acceptable carrier.

11. A method for inhibiting cannabinoid CB₁ receptor in a mammal, which comprises administering the compound of formula (I) of claim 1 to the mammal.

12. A method for preventing or treating obesity and obesity-related metabolic disorders in a mammal, which comprises administering the compound of formula (I) of claim 1 to the mammal.
